(19) United States
(12) Patent Application Publication
(10) Pub. No.: US 2026/0337211 A1
(43) Pub. Date: Oct. 29, 2026

(54) IP5 SUBSTITUTED COMPOUNDS

(71) Applicant: SANIFIT THERAPEUTICS, S.A., Palma de Mallorca (ES)

(72) Inventors: Carolina SALCEDO ROCA, Palma de Mallorca (ES); María del Mar PÉREZ FERRER, Palma de Mallorca (ES); Miquel David FERRER REYNÉS, Palma de Mallorca (ES); Mohamad Firas BASSISSI, Palma de Mallorca (ES); Carme SERRA COMAS, Palma de Mallorca (ES); Juan Lorenzo CATENA RUIZ, Palma de Mallorca (ES); Amadeu LLEBARIA SOLDEVILA, Palma de Mallorca (ES); Joan PERELLÓ BESTARD, Palma de Mallorca (ES)

(21) Appl. No.: 19/099,411

(22) PCT Filed: Jul. 31, 2023
(86) PCT No.: PCT/EP2023/071141

§ 371 Date: Jan. 28, 2025

(30) Foreign Application Priority Data
Jul. 29, 2022 (EP) 22382737.9

(51) Int. Cl. C07F 9/117; A61K 31/6615; A61P 9/00; A61P 19/00

(52) U.S. Cl. CPC C07F 9/117; A61K 31/6615; A61P 9/00; A61P 19/00

(57) ABSTRACT

The present invention provides IP5 substituted compounds of general formula I, their methods of synthesis and their uses. The IP5 substituted compounds of the invention are characterized for having —O(alkyl)ₙX, and —O(alkyl)_{y}Cy(alkyl)_{y}′-Z chains in positions R₁, R₂, R₄, and R₅. Also provided are methods, pharmaceutical compositions and formulations, methods of use, articles of manufacture, and kits for the treatment of diseases and conditions such as pathological crystallization-related diseases and conditions.

### TECHNICAL FIELD

The present invention relates to IP5 substituted compounds, their processes of synthesis, and their uses.

### STATE OF THE ART

Crystallization and deposition of calcium salts are common phenomena. Generally, these processes are physiologically necessary for the well-being of the individual. However, on occasions, they may become abnormal and/or pathological causing a wide variety of diseases and disorders affecting the vascular and renal systems, often with devastating consequences (Khan S, et al., Pathological crystallization of calcium oxalate and calcium phosphate, in Hydroxyapatite and Related Materials, 1^{st} Ed., Brown P, et al., Eds. (CRC Press, Boca Raton, FL, US, 1994, Ch. 5). Hydroxyapatite (HAP), a calcium phosphate of formula Ca₁₀(PO₄)₆(OH)₂, has been identified as an agent involved in pathological crystallization (Grases F, et al., Cir J 2007; 71:1152-1156).

Several crystallization inhibitors that may affect HAP formation and growth, such as pyrophosphate, bisphosphonates (e.g., etidronate, alendronate, ibandronate), and phytate, have been described previously in the art (Lomashvili K, et al., J Am Soc Nephrol 2004; 15:1392-1401, Bevilacqua M, et al., Lupus 2005; 14:773-779, Price P, et al., Arterioscler Thromb Vasc Biol 2001; 21:817-824, Price P, et al., J Nutr 2001; 131:2910-2915, Grases F, et al., Front Biosci 2006; 11:136-142). More recently, a number of phytate-related compounds, such as pentaphosphate myo-inositol (IP5) substituted derivatives, have been proposed for various applications including the inhibition of HAP and/or the prevention and treatment of pathological crystallization, among others. For instance, WO2011064559 and Wang H, et al., Chem Biol 2014; 21:689-699 reveal the use of IP5 derivatives for the treatment of cancer. WO2009061393 and Huang X, et al., ACS Appl Mater Interfaces 2017; 9:10435-10445 describes the use of IP5 derivatives as biological tools and calcium chelators, respectively. In addition, WO2021219135, WO2017098047, and WO2020058321 describe the use of these compounds for the treatment of pathologies associated with calcium. However, there is still a need in the art for new IP5 substituted derivatives that could be effective in inhibiting HAP formation and growth and/or pathological crystallization.

### BRIEF SUMMARY OF THE INVENTION

The present invention discloses a compound of general formula I:

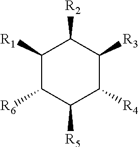

a pharmaceutically acceptable salt thereof, or a combination thereof, wherein
(i) R₁, R₂, R₃, R₅, and R₆ independently represent —OPO₃H₂ and R₄ is a substituent group of formula II or formula III, or R₁, R₂, R₃, R₄, and R₅ independently represent OPO₃H₂ and R₆ is a substituent group of formula II or formula III,

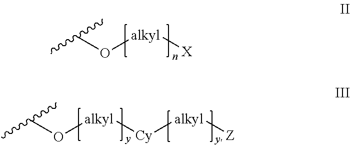

(ii) R₁, R₃, R₄, R₅, and R₆ independently represent —OPO₃H₂ and R₂ is a substituent group of formula II or formula III,
(iii) R₁, R₂, R₃, R₄, and R₆ independently represent —OPO₃H₂ and R₅ is a substituent group of formula II or formula III,
(iv) R₂, R₃, R₄, R₅, and R₆ independently represent —OPO₃H₂ and R₁ is a substituent group of formula II; or R₁, R₂, R₄, R₅, and R₆ independently represent —OPO₃H₂ and R₃ is a substituent group of formula II or formula III,
wherein, for formula II, n is an integer between 1 and 30, wherein the terminal group X is selected from the group consisting of —H, —OR, —NRR′, —COOR, —CONRR′, —NHCOR, —NHCOOR, —OCONR, —NHSO₂R, —NHCONRR′, halogen, —CF₃, alkyl, alkenyl, alkynyl, carbocycle (saturated or unsaturated), and heterocycle (saturated or unsaturated), and wherein R and R′ are H or an alkyl group, and wherein, for formula III, y and y′ are an integer between 0 and 10, wherein Cy is a cyclic linker, wherein the terminal group Z is selected from the group consisting of alkyl, —COR, —OR, —NRR′, —COOR, —CONRR′, —NHCOR, —NHCOOR, —OCONR —NHSO₂R, —NHCONRR′, halogen, and —CF₃, and wherein R and R′ are H or an alkyl group.

In some aspects, the compound of formula I is selected from the group consisting of Compounds 1 to Compound 53.

In some aspects, the pharmaceutically acceptable salt is a sodium or magnesium salt. In some aspects, the sodium salt is a tetrasodium salt, pentasodium salt, hexasodium salt, heptasodium salt, octasodium salt, nonasodium salt, decasodium salt or undecasodium salt.

The invention also provides a pharmaceutical composition comprising a compound of formula I disclosed above and at least one pharmaceutically acceptable excipient.

Also provided is a method to inhibit the formation or growth of calcium salts/crystals (e.g., calcium phosphates, hydroxyapatite (HAP)) in a subject in need thereof comprising administering to the subject an effective amount of a compound of formula I, or a pharmaceutical composition of disclosed herein.

Additionally, the invention provides a method to treat or prevent a disease or condition associated with pathological crystallization in a subject in need thereof comprising administering to the subject an effective amount of a compound of formula I, or a pharmaceutical composition of disclosed herein.

Also provided is a method to inhibit the progression of a crystallization process in a subject in need thereof comprising administering to the subject an effective amount of a compound of formula I, or a pharmaceutical composition of disclosed herein.

The invention also provides a method to recover or increase blood perfusion in a subject in need thereof comprising administering to the subject an effective amount of a compound of formula I, or a pharmaceutical composition of disclosed herein.

In some aspects, the subject is human. In some aspects, the administration is topical, enteral or parenteral. In some aspects, the parenteral administration is intravenous. In some aspects, the intravenous administration is by bolus injection or by infusion.

The present invention also provides a kit or article of manufacture comprising at least one compound of formula I or a pharmaceutical composition comprising a compound of formula I and instructions for administration according to any method disclosed herein. In some aspects, the kit or article of manufacture may also comprise at least one compound selected from the group consisting of the compounds listed in Table 1.

In some aspects, the present invention provides additionally a method for the manufacture of a compound of formula I (e.g., Compound 1 to Compound 53) which comprises using at least one compound selected from the group consisting of the compounds listed in Table 1.

### BRIEF DESCRIPTION OF THE DRAWINGS

FIG. 1A and FIG. 1B present representative structures of Compounds I_A: IP5-4 substituted compounds.

FIG. 2A and FIG. 2B present representative structures of Compounds I_B: IP5-2 substituted compounds.

FIG. 3 presents representative structures of Compounds I_C: IP5-5 substituted compounds.

FIG. 4 presents representative structures of Compounds I_D: IP5-1 substituted compounds.

FIG. 5 is a schematic representation of synthesis Scheme 1.

FIG. 6 is a schematic representation of synthesis Scheme 2.

FIG. 7A and FIG. 7B are schematic representations of syntheses Scheme 3 and Scheme 6, respectively.

FIG. 8A and FIG. 8B are schematic representations of syntheses Scheme 4 and Scheme 7, respectively.

FIG. 9A and FIG. 9B are schematic representations of syntheses Scheme 5 and Scheme 8, respectively.

### DETAILED DESCRIPTION OF THE INVENTION

The present invention provides IP5 substituted compounds, their methods of synthesis and their uses. In some aspects, the IP5 substituted compound is a compound of general formula I:

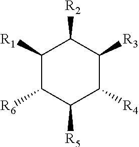

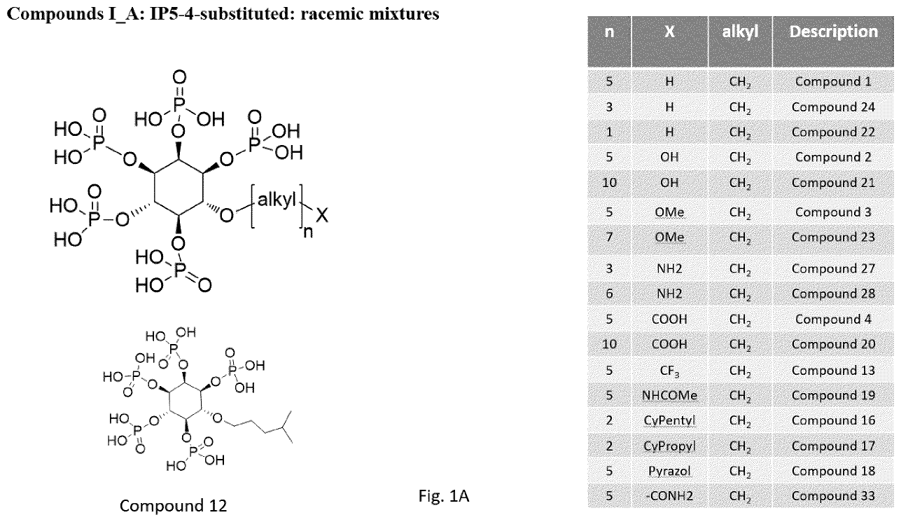

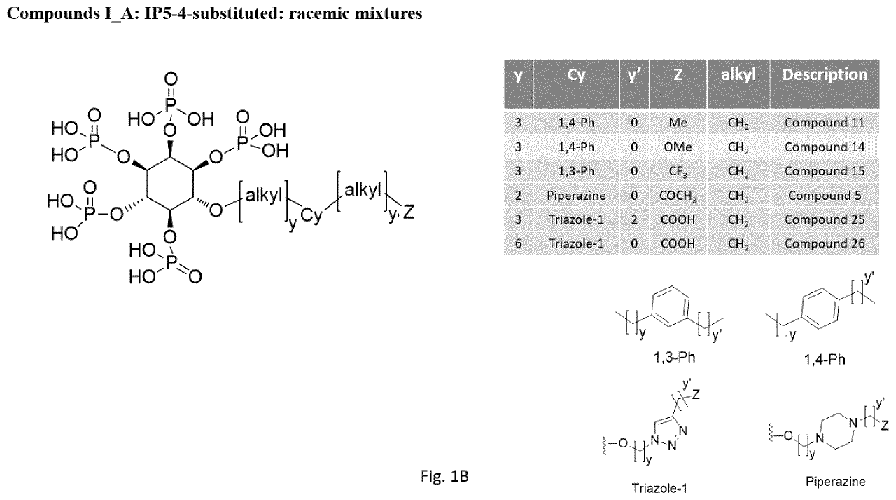

a pharmaceutically acceptable salt thereof, or a combination thereof, wherein
(i) R₁, R₂, R₃, R₅, and R₆ independently represent —OPO₃H₂ and R₄ is a substituent group of formula II or formula III, or R₁, R₂, R₃, R₄, and R₅ independently represent —OPO₃H₂ and R₆ is a substituent group of formula II or formula III, (See FIG. 1A, 1B)

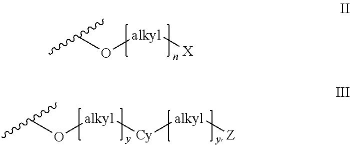

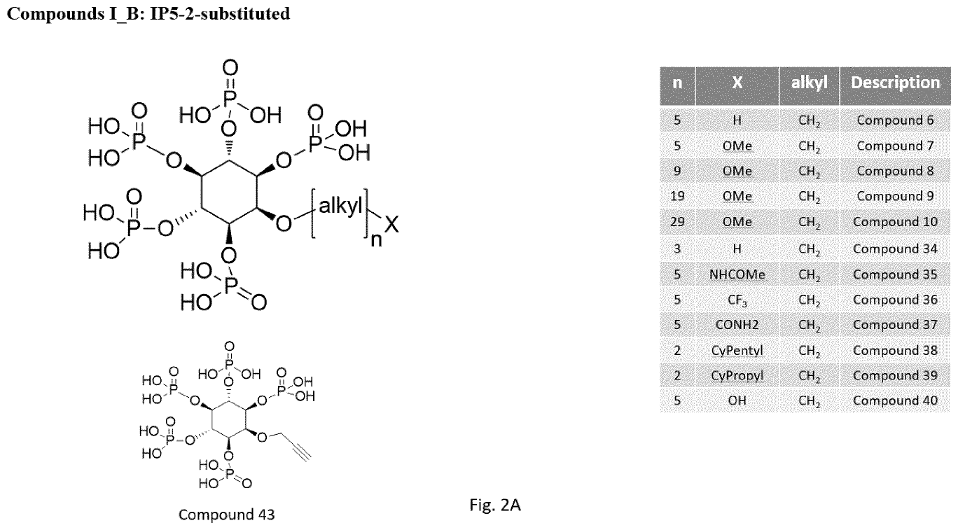

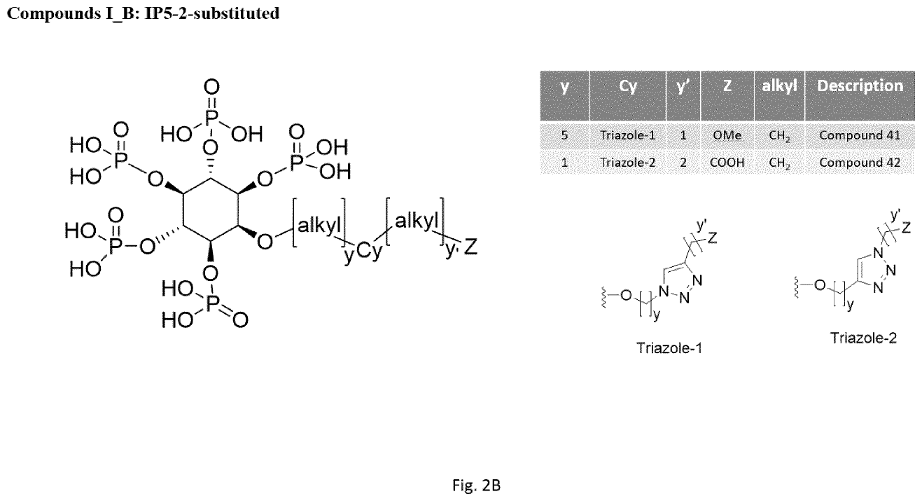

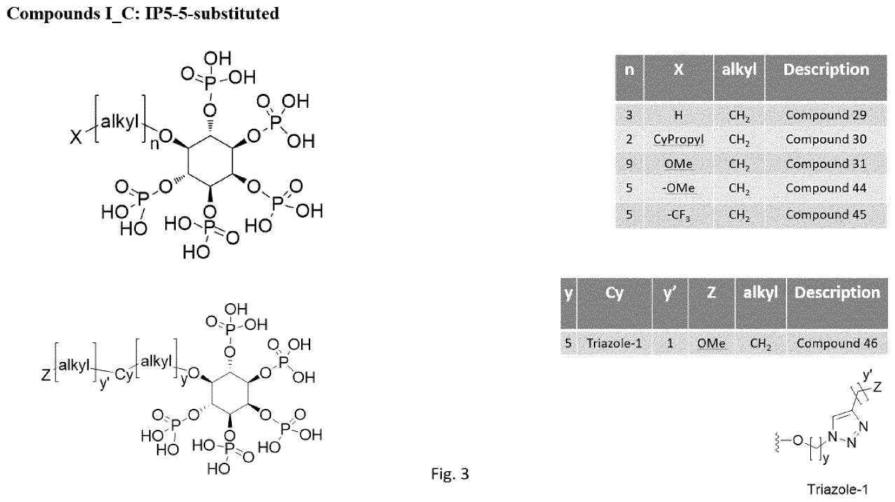

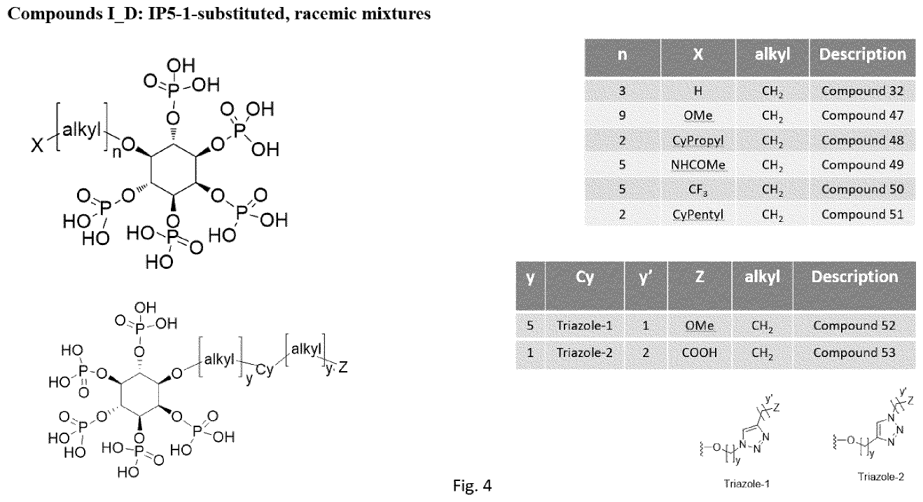

(ii) R₁, R₃, R₄, R₅, and R₆ independently represent —OPO₃H₂ and R₂ is a substituent group of formula II or formula III, (See FIG. 2A, 2B)
(iii) R₁, R₂, R₃, R₄, and R₆ independently represent —OPO₃H₂ and R₅ is a substituent group of formula II or formula III, (See FIG. 3)
(iv) R₂, R₃, R₄, R₅, and R₆ independently represent —OPO₃H₂ and R₁ is a substituent group of formula II; or R₁, R₂, R₄, R₅, and R₆ independently represent —OPO₃H₂ and R₃ is a substituent group of formula II or formula III (See FIG. 4), wherein, for formula II, n is an integer between 1 and 30, wherein the terminal group X is selected from the group consisting of —H, —OR, —NRR′, —COOR, —CONRR′, —NHCOR, —NHCOOR, —OCONR, —NHSO₂R, —NHCONRR′, halogen, —CF₃, alkyl, alkenyl, alkynyl, carbocycle (saturated or unsaturated), and heterocycle (saturated or unsaturated), and wherein R and R′ are H or an alkyl group, and wherein, for formula III, y and y′ are an integer between 0 and 10, wherein Cy is a cyclic linker, wherein the terminal group Z is selected from the group consisting of alkyl, —COR, —OR, —NRR′, —COOR, —CONRR′, —NHCOR, —NHCOOR, —OCONR —NHSO₂R, —NHCONRR′, halogen, and —CF₃, and wherein R and R′ are H or an alkyl group. In some aspects, the IP5 substituted compound is of formula IV, V, VI, VII, VII, VIII, IX, X or XI.

In some aspects, the IP5 substituted compound is a sodium or magnesium salt. In some aspects, the IP5 substituted compound is a tetrasodium salt, pentasodium salt, hexasodium salt, heptasodium salt, octasodium salt, nonasodium salt, decasodium salt or undecasodium salt.

Also provided are methods, pharmaceutical compositions and formulations, methods of use, articles of manufacture, and kits for the treatment of diseases and conditions such as pathological crystallization-related diseases and conditions.

In order that the present invention can be more readily understood, certain terms are first defined below. As used in this application, except as otherwise expressly provided herein, each of the following terms shall have the meaning set forth below. Additional definitions are set forth throughout the application.

### I. Definitions

The invention includes aspects in which exactly one member of the group is present in, employed in, or otherwise relevant to a given product or process. The invention includes aspects in which more than one, or all of the group members are present in, employed in, or otherwise relevant to a given product or process.

Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2^{nd} Ed., 2002, CRC Press; The Dictionary of Cell and Molecular Biology, 3^{rd} Ed., 1999, Academic Press; and the Oxford Dictionary of Biochemistry and Molecular Biology, Revised, 2000, Oxford University Press, provide one of skill with a general dictionary of many of the terms used in this invention.

Units, prefixes, and symbols are denoted in their Systeme International d'Unités (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention.

Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

About: The term “about” as used herein to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the terms can mean up to an order of magnitude or up to 5-fold of a value.

When particular values or compositions are provided in the application and claims, unless otherwise stated, the meaning of “about” should be assumed to be within an acceptable error range for that particular value or composition. When the term “about” is used in conjunction with a numerical range, it modifies that range by extending the boundaries above and below the numerical values set forth. Thus, “about 10-20” means “about 10 to about 20.” In general, the term “about” can modify a numerical value above and below the stated value by a variance of, e.g., 10 percent, up or down (higher or lower).

And/or: “And/or” where used herein is to be taken as specific invention of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

Bolus administration: As used herein, the terms “bolus administration” and “bolus injection” refer a fast intravenous injection lasting less than 10 seconds, or an intravenous infusion lasting less than 3 minutes.

Comprising: It is understood that wherever aspects are described herein with the language “comprising,” otherwise analogous aspects described in terms of “consisting of” and/or “consisting essentially of” are also provided.

Compound: As used herein, the term “compound,” is meant to include any and all free bases, isomers, and isotopes of the structure depicted. As used herein, the term “isomer” means any geometric isomer, tautomer, zwitterion, stereoisomer, enantiomer, or diastereomer of a compound. Compounds can include one or more chiral centers and/or double bonds and can thus exist as stereoisomers, such as double-bond isomers (i.e., geometric E/Z isomers) or diastereomers (e.g., enantiomers (i.e., (+) or (−)) or cis/trans isomers). The present invention encompasses any and all isomers of the compounds described herein, including stereomerically pure forms (e.g., geometrically pure, enantiomerically pure, or diastereomerically pure) and enantiomeric and stereoisomeric mixtures (e.g., racemates). Enantiomeric and stereometric mixtures of compounds and means of resolving them into their component enantiomers or stereoisomers are well-known. A compound, salt, or complex of the present invention can be prepared in combination with solvent or water molecules to form solvates and hydrates by routine methods. In some aspects, the term compound is used to refer to an IP5 substituted compound of the present invention.

Effective amount: As used herein, the term “effective amount” of a therapeutic agent, in reference to (i) an IP5 substituted compound of the present invention, (ii) any dosage form, pharmaceutical composition, or formulation disclosed herein comprising at least one IP5 substituted compound of the present invention, or (iii) a combination of an IP5 substituted compound of the present invention with one or more additional therapeutic agents), is that amount sufficient to effect beneficial or desired results. In some aspects, the beneficial or desired results are, for example, clinical results, and, as such, an “effective amount” depends upon the context in which it is being applied. The term “effective amount” can be used interchangeably with “effective dose,” “therapeutically effective amount,” or “therapeutically effective dose.”

The term effective amount relates to the specific use of an IP5 substituted compound. For example, when an IP5 substituted compound is used to inhibit the formation or growth of a calcium salt/crystal (e.g., a calcium phosphate, HAP), an effective amount would be an amount of the IP5 substituted compound capable of achieving the desired effect (e.g., the reduction of HAP crystallization/formation in blood serum or plasma).

Enteral administration: As used herein, the term “enteral administration” and the related term “enterally” refer to any administration of an IP5 substituted compound of the present invention or a pharmaceutical composition comprising said compound via the gastrointestinal tract. Enteral administration includes, but is not limited to, the oral, sublingual, and rectal routes of administration.

Prophylaxis: As used herein, the term “prophylaxis” refers to a measure taken to maintain health and prevent or delay the onset of a disease or condition or to mitigate its extent and/or severity of the symptoms. Thus, a prophylactic use of a therapeutic agent disclosed herein, for example, (i) an IP5 substituted compound of the present invention, or (ii) a combination thereof, or (iii) any dosage form comprising at least one IP5 substituted compound of the present invention, or (iv) any formulation comprising at least one IP5 substituted compound of the present invention, or a (v) combination of an IP5 substituted compound of the present invention with one or more additional therapeutic agents, corresponds to that amount sufficient to effect beneficial or desired results.

Ranges: As described herein, any concentration range, percentage range, ratio range or integer range is to be understood to include the value of any integer within the recited range and, when appropriate, fractions thereof (such as one tenth and one hundredth of an integer), unless otherwise indicated.

IP5 substituted compounds of the present invention: As used herein, the term “IP5 substituted compound of the present invention” and grammatical variants thereof refers to a compound of formula I wherein its substituents are those disclosed in compound Families I_A, I_B, I_C, and I_D described in detail below, and salts thereof (e.g., pharmaceutically acceptable salts thereof). In some aspects, the term IP5 substituted compound of the present invention encompasses Compounds 1 to 53, any salt thereof (e.g., a sodium salt), and any combination thereof. In some aspects, the term IP5 substituted compound of the present invention encompasses a compound of formula I which is an intermediate in the synthesis of Compounds 1 to 53, e.g., a compound selected from the group consisting of the compounds listed in Table 1, any salt thereof (e.g., a sodium salt), and any combination thereof. In some aspects, the term IP5 substituted compound of the present invention encompasses a compound of formula I which is Compound 1 to Compound 53 and a compound selected from the group consisting of the compounds listed in Table 1, any salt thereof (e.g., a sodium salt), and any combination thereof.

Group consisting of Compound 1 to Compound 53: In the context of the present invention, references to a “group consisting of Compound 1 to Compound 53”, refers to a group of compounds that comprises Compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, and 53. In some aspects, the group consisting of Compound 1 to Compound 53 also comprises combinations thereof. In some aspects, a combination of compounds from the group consisting of Compound 1 to Compound 53 can comprise 2, 3, 4, 5, 6, 7, 8, 9, 10 or more compounds from the group consisting of Compound 1 to Compound 53.

Group consisting of the compounds listed in Table 1: In the context of the present invention, references to “Group consisting of the compounds listed in Table 1”, refers to a group of intermediate compounds used, e.g., for the synthesis of an IP5 substituted compound of the present invention (e.g., a compound selected from the group consisting of Compound 1 to Compound 53) i.e., Intermediates II_A, II_B, II_B′, II_C, and II_D, Intermediates III_A, III_B, III_C, and III_D, Intermediates IV_A, IV_B, IV_C, and IV_D, Intermediates V_A, V_B, V_C and V_D, Intermediates VI_A, Intermediates VII_A, Intermediates VIII_B and VIII_B′, Intermediates IX_D and Intermediates X_D. In some aspects, the group consisting of the compounds listed in Table 1 also comprises combinations thereof. In some aspects, a combination of compounds from the group consisting of the compounds listed in Table 1 can comprise 2, 3, 4, 5, 6, 7, 8, 9, 10 or more compounds from the group consisting of the compounds listed in Table 1.

Non-bolus administration: As used herein, the terms “non-bolus type” and “non-bolus administration” refer to an intravenous injection lasting 10 or more seconds, or an intravenous infusion lasting 3 or more minutes.

Parenteral administration: As used herein, the term “parenteral administration” and the related term “parenterally” refer to the administration of an IP5 substituted compound of the present invention characterized by the physical breaching of a tissue of a subject and the administration of the compound through said breach in the tissue. Parenteral administration includes, but is not limited to, the administration of an IP5 substituted compound of the present invention (e.g., a compound selected from the group consisting of Compound 1 to Compound 53) or a pharmaceutical composition comprising the compound, by the application of the compound or the composition through, for instance, a surgical incision or through a tissue-penetrating non-surgical wound. In particular, parenteral administration includes, but is not limited to, the epidural, intraarterial, intradermal, intrathecal, intramuscular, intraperitoneal, intrasternal injection, intravascular, intravenous, intravenous infusion, spinal, subcutaneous, and subcutaneous depot routes of administration.

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain aspects, the mammal is a human subject. In other aspects, a subject is a human patient. In a particular aspect, a subject is a human patient with a pathological crystallization or at risk of having pathological crystallizations.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic agent: As used herein, the term “therapeutic agent” is used in a broad sense to include a composition comprising an IP5 substituted compound of the present invention that can provide a significant therapeutic benefit to a subject in need thereof. In some aspects, the subject in need thereof is a subject suffering or at risk of developing a disease or condition associated to pathological crystallization (e.g., a calcium phosphate or HAP crystallization). Thus, in general, a therapeutic agent according to the present invention can be an IP5 substituted compound of the present invention, alone or in combination with one or more additional therapeutic agents, that is administered in an amount sufficient to effect beneficial or desired results.

The term therapeutic agent also encompasses prophylactic, diagnostic or imaging agents comprising an IP5 substituted compound of the present invention, wherein the therapeutic agent is administered (i.e., topically, enterally or parenterally). Therapeutic agents of the present invention include agents that inhibit the formation or growth of calcium salts/crystals (e.g., calcium phosphates, HAP) and/or can ameliorate and/or prevent any symptom associated with pathological crystallization.

Topical administration: As used herein, the term “topical administration” and the related term “topically” refer to any administration of an IP5 substituted compound of the present invention or a pharmaceutical composition comprising said compound by applying the compound or composition to a particular place on or in the body, such as the skin or a mucous membrane. Topical administration includes, but is not limited to, the aural, cutaneous, nasal, transdermal, urethral, vaginal, and urethral routes of administration.

Treating, treatment, therapy: As used herein, the terms “treating” or “treatment” or “therapy” refer to partially or completely alleviating, ameliorating, improving, relieving, delaying onset of, inhibiting progression of, reducing severity of, reducing incidence of one or more symptoms or features of disease, or any combination thereof.

A treatment comprising an IP5 substituted compound of the present invention can be administered to a subject who does not exhibit signs of a disease, disorder, and/or condition, and/or to a subject who exhibits only early signs of a disease, disorder, and/or condition for the purpose of, e.g., (i) decreasing the risk of developing a pathology associated with the disease, disorder, and/or condition, (ii) delaying the onset of the disease, disorder, and/or condition, or a pathology associated with said disease, disorder, and/or condition, or (iii) mitigating the symptoms and/or sequels of the disease, disorder, and/or condition or a pathology associated with said disease, disorder, and/or condition.

Thus, in general, the term “treatment” refers to countering the effects caused as a result of the disease or pathological condition of interest in a subject including (i) inhibiting the disease or pathological condition, in other words, slowing or stopping the development or progression thereof; (ii) relieving the disease or pathological condition, in other words, causing said disease or pathological condition, or the symptoms thereof, to regress; (iii) stabilizing the disease or pathological condition, and (iv) any combination thereof.

ug, uM, uL: As used herein, the terms “ug,” “uM,” and “uL” are used interchangeably with “μg,” “μM,” and “μL” respectively.

### II. IP5 Substituted Compounds

The present invention provides IP5 substituted compounds, their methods of synthesis and their uses. In some aspects, the IP5 substituted compound is a compound of general formula I:

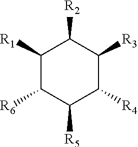

a pharmaceutically acceptable salt thereof, or a combination thereof, wherein
R₁, R₂, R₃, R₅, and R₆ independently represent —OPO₃H₂ and R₄ is a substituent group of formula II or formula III, or R₁, R₂, R₃, R₄, and R₅ independently represent —OPO₃H₂ and R₆ is a substituent group of formula II or formula III,

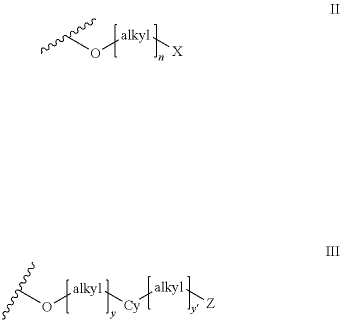

R₁, R₃, R₄, R₅, and R₆ independently represent —OPO₃H₂ and R₂ is a substituent group of formula II or formula III,
R₁, R₂, R₃, R₄, and R₆ independently represent —OPO₃H₂ and R₅ is a substituent group of formula II or formula III,
R₂, R₃, R₄, R₅, and R₆ independently represent —OPO₃H₂ and R₁ is a substituent group of formula II; or R₁, R₂, R₄, R₅, and R₆ independently represent —OPO₃H₂ and R₃ is a substituent group of formula II or formula III,
wherein, for formula II, n is an integer between 1 and 30, wherein the terminal group X is selected from the group consisting of —H, —OR, —NRR′, —COOR, —CONRR′, —NHCOR, —NHCOOR, —OCONR, —NHSO₂R, —NHCONRR′, halogen, —CF₃, alkyl, alkenyl, alkynyl, carbocycle (saturated or unsaturated), and heterocycle (saturated or unsaturated), and wherein R and R′ are H or an alkyl group, and wherein, for formula III, y and y′ are an integer between 0 and 10, wherein Cy is a cyclic linker, wherein the terminal group Z is selected from the group consisting of alkyl, —COR, —OR, —NRR′, —COOR, —CONRR′, —NHCOR, —NHCOOR, —OCONR —NHSO₂R, —NHCONRR′, halogen, and —CF₃, and wherein R and R′ are H or an alkyl group. In some aspects, the IP5 substituted compound is of formula IV, V, VI, VII, VII, VIII, IX, X or XI.

In some aspects, not all negative charges in an IP5 substituted compound of the present invention are compensated by charges in positively charged ions (e.g., Na⁺, Ca⁺⁺, Mg⁺⁺). Accordingly, an IP5 substituted compound of the present invention can be, for example, a tetraionic salt (e.g., tetrasodium salt), a pentaionic salt (e.g., pentasodium salt), a hexaionic salt (e.g., hexasodium salt), a heptaionic salt (e.g., heptasodium salt), an octaionic salt (e.g., octasodium salt), a nonaionic salt (e.g., nonasodium salt), a decaionic salt (e.g., decasodium salt) or a undecaionic salt (e.g., undecasodium salt). In some aspects, the presence of additional negatively charges group in a IP5 substituted compounds can lead to the formation of complexes with additional ions. In some aspects, the IP5 substituted compound of the present invention is a sodium salt. In some aspects, the IP5 substituted compound is a tetrasodium salt, pentasodium salt, hexasodium salt, heptasodium salt, octasodium salt, nonasodium salt, decasodium or undecasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. Formula I and the rest of formulas presented in the invention are meant to encompass any isomers of the compounds covered thereby.

The term “alkenyl” or “alkenyl chain” in the context of the present invention refers to a linear or branched alkyl chain (e.g., containing between 2 and 10 carbon atoms) containing one or more double bonds, either substituted or non-substituted. Examples include, among others, ethenyl, 1-propenyl, 2-propenyl, isopropenyl. 1-butenyl, 2-butenyl, 3-butenyl, and 1,3-butadienyl.

The term “alkyl” or “alkyl chain” in the context of the present invention refers to a hydrocarbon moiety, which can be saturated, partially unsaturated, linear, branched, cyclic or cyclic with linear or branched side chains containing from 1 to 30 carbon atoms. Examples include but are not limited are to C₁-C₄ alkyls such as methyl, ethyl, propyl, isopropyl, n- or isobutyl, and cycloalkyl such as cyclohexyl. The term alkyl can extend to alkyl groups linked or bridged by hetero atoms. Hetero atoms in the context of the present invention are nitrogen (N), sulfur (S), oxygen (O), and halogen.

The term “alkynyl” or “alkynyl chain” in the context of the present invention refers to a linear or branched alkyl chain (e.g., containing between 2 and 10 carbon atoms) containing one or more triple bonds, either substituted or non-substituted. Examples include, among others, ethynyl, propynyl, 1-butynyl, and 3-butynyl.

An “amine function” or “amine group” is a function NRR′, with R and R′ selected independently, e.g., from hydrogen (—H) and an alkyl group such as an C₁-Cₙ alkyl, wherein n is and integer between 0 and 30.

A “hydroxy function” or “hydroxy group” is OH.

A “carboxylic acid function” or “carboxylic acid group” is COOH or its anion, COO⁻.

A “carboxylic amide” is CONRR′ or NCOR, with R and R′ selected independently, e.g., from hydrogen (—H) and an alkyl group such as an C₁-Cₙ alkyl, wherein n is and integer between 0 and 20.

A “carbocycle” refers to a three- to 10-membered carbocyclic ring that can be saturated, partially unsaturated or aromatic (e.g., phenyl, cyclopentyl, cyclopropyl) and which is bound to the rest of the molecule via any available C atom.

A “heterocycle” refers to a three- to 10-membered cyclic ring containing at least one heteroatom selected from among N, O, and S, that can be saturated, partially unsaturated or aromatic (e.g., triazole, piperazine, pyrazole) and which is bound to the rest of the molecule via any available C atom or N atom. The term includes heterocycle rings substituted with one or more halogen atoms.

A “Cy” refers to a cyclic linker comprising a carbocycle or a heterocycle. Examples of carbocycles and heterocycles include, among others, 1,3-phenyl, 1,4-phenyl, naphthyl, thienyl, furyl, pyrrolyl, thiazolyl, isothiazolyl, imidazolyl, pyrazolyl, 1,2,3-triazolyl, 1,2,4-triazolyl, tetrazolyl, 1,3,4-thiadiazolyl, 1,2,4-thiadiazolyl, pyridyl, piperazyl, pyrazinyl, pyrimidinyl, pyridazinyl, benzimidazolyl, benzofuranyl, isobenzofuranyl, indolyl, isoindolyl, benzothiophenyl, benzothiazolyl, cyclopropyl, cyclobutyl, cyclopentyl, cyclohexyl, cycloheptyl, azetidinyl, and aziridinyl.

A “halogen” group refers to fluorine, chlorine, bromine or iodine.

The term “—OPO₃²⁻” in the context of the present invention refers also indistinctly to —OPO₃H⁻ and —OPO₃H₂.

In some aspects, the IP5 substituted compounds of the present invention or intermediate compounds disclosed herein can be detected and/or quantified using the methods disclosed in U.S. Pat. No. 9,612,250. See also, U.S. Pat. Nos. 8,377,909, 8,778,912 and US20070066574.

The IP5 substituted compounds of the present invention can be present in any form commonly used in pharmaceutical technology. Particular aspects include, but are not limited to, the sodium salt, magnesium salt, potassium salt, ammonium salt, free acid, or a mixture of the preceding forms. Other pharmaceutically acceptable salts are known to the skilled artisan and can be obtained by methods previously described (Haynes M, et al., J. Pharmaceutical Sci. 2005; 94:2111-2120.

In some aspects, an IP5 substituted compound of the present invention comprises or consists of an IP5 substituted compound selected from the group consisting of Compounds 1 to 53 and combinations thereof. In some aspects, an IP5 substituted compound of the present invention comprises or consists of Compound 1. In some aspects, an IP5 substituted compound of the present invention comprises or consists of Compound 2. In some aspects, an IP5 substituted compound of the present invention comprises or consists of Compound 3. In some aspects, an IP5 substituted compound of the present invention comprises or consists of Compound 4. In some aspects, an IP5 substituted compound of the present invention comprises or consists of Compound 5. In some aspects, an IP5 substituted compound of the present invention comprises or consists of Compound 6. In some aspects, an IP5 substituted compound of the present invention comprises or consists of Compound 7. In some aspects, an IP5 substituted compound of the present invention comprises or consists of Compound 8. In some aspects, an IP5 substituted compound of the present invention comprises or consists of Compound 9. In some aspects, an IP5 substituted compound of the present invention comprises or consists of Compound 10. In some aspects, an IP5 substituted compound of the present invention comprises or consists of Compound 11. In some aspects, an IP5 substituted compound of the present invention comprises or consists of Compound 12. In some aspects, an IP5 substituted compound of the present invention comprises or consists of Compound 13. In some aspects, an IP5 substituted compound of the present invention comprises or consists of Compound 14. In some aspects, an IP5 substituted compound of the present invention comprises or consists of Compound 15. In some aspects, an IP5 substituted compound of the present invention comprises or consists of Compound 16. In some aspects, an IP5 substituted compound of the present invention comprises or consists of Compound 17. In some aspects, an IP5 substituted compound of the present invention comprises or consists of Compound 18. In some aspects, an IP5 substituted compound of the present invention comprises or consists of Compound 19. In some aspects, an IP5 substituted compound of the present invention comprises or consists of Compound 20. In some aspects, an IP5 substituted compound of the present invention comprises or consists of Compound 21. In some aspects, an IP5 substituted compound of the present invention comprises or consists of Compound 22. In some aspects, an IP5 substituted compound of the present invention comprises or consists of Compound 23. In some aspects, an IP5 substituted compound of the present invention comprises or consists of Compound 24. In some aspects, an IP5 substituted compound of the present invention comprises or consists of Compound 25. In some aspects, an IP5 substituted compound of the present invention comprises or consists of Compound 26. In some aspects, an IP5 substituted compound of the present invention comprises or consists of Compound 27. In some aspects, an IP5 substituted compound of the present invention comprises or consists of Compound 28. In some aspects, an IP5 substituted compound of the present invention comprises or consists of Compound 29. In some aspects, an IP5 substituted compound of the present invention comprises or consists of Compound 30. In some aspects, an IP5 substituted compound of the present invention comprises or consists of Compound 31. In some aspects, an IP5 substituted compound of the present invention comprises or consists of Compound 32. In some aspects, an IP5 substituted compound of the present invention comprises or consists of Compound 33. In some aspects, an IP5 substituted compound of the present invention comprises or consists of Compound 34. In some aspects, an IP5 substituted compound of the present invention comprises or consists of Compound 35. In some aspects, an IP5 substituted compound of the present invention comprises or consists of Compound 36. In some aspects, an IP5 substituted compound of the present invention comprises or consists of Compound 37. In some aspects, an IP5 substituted compound of the present invention comprises or consists of Compound 38. In some aspects, an IP5 substituted compound of the present invention comprises or consists of Compound 39. In some aspects, an IP5 substituted compound of the present invention comprises or consists of Compound 40. In some aspects, an IP5 substituted compound of the present invention comprises or consists of Compound 41. In some aspects, an IP5 substituted compound of the present invention comprises or consists of Compound 42. In some aspects, an IP5 substituted compound of the present invention comprises or consists of Compound 43. In some aspects, an IP5 substituted compound of the present invention comprises or consists of Compound 44. In some aspects, an IP5 substituted compound of the present invention comprises or consists of Compound 45. In some aspects, an IP5 substituted compound of the present invention comprises or consists of Compound 46. In some aspects, an IP5 substituted compound of the present invention comprises or consists of Compound 47. In some aspects, an IP5 substituted compound of the present invention comprises or consists of Compound 48. In some aspects, an IP5 substituted compound of the present invention comprises or consists of Compound 49. In some aspects, an IP5 substituted compound of the present invention comprises or consists of Compound 50. In some aspects, an IP5 substituted compound of the present invention comprises or consists of Compound 51. In some aspects, an IP5 substituted compound of the present invention comprises or consists of Compound 52. In some aspects, an IP5 substituted compound of the present invention comprises or consists of Compound 53.

The IP5 substituted compounds of the present invention are disclosed in myo form.

The present invention also provides chemical intermediate compounds useful in the preparations of IP5 substituted compound of the present invention (e.g., Compound 1 to Compound 53). In some aspects, such intermediates are the compounds listed in Table 1. An intermediate compound disclosed herein can be converted to an IP5 substituted compound of the present invention by utilizing the procedures described herein. Thus, the present invention provides methods to produce an IP5 substituted compound of the present invention (e.g., a compound selected from the group consisting of Compounds 1 to 53) comprising utilizing an intermediate compound selected from the group consisting of the compounds listed in Table 1. The present invention also provides methods of producing the intermediate compounds disclosed herein. Accordingly, the present invention provides methods of producing intermediates compounds selected from the group consisting of the compounds listed in Table 1 for producing IP5 substituted compounds of the present invention (e.g., a compound selected from the group consisting of Compounds 1 to 53).

In some aspects, an IP5 substituted compound of the present invention comprises or consists of a compound of the following formula:

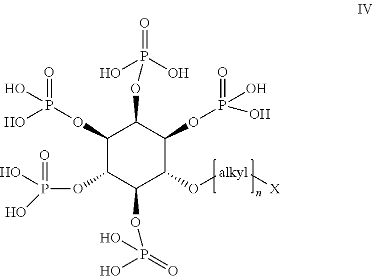

wherein n is an integer between 1 and 30, alkyl is CH₂ and X is —H, —OH, —OMe, pyrazole, triazole, —COOH, CONRR′, —NHCOR, —NHCOOR, —OCONR, —NHSO₂R, —NHCONRR′, —CF₃, alkyl, cyclopropane, cyclopentane or pyrazole. See FIG. 1A. In some aspects, n is an integer between 1 and 20. In some aspects, n is an integer between 1 and 10. In some aspects, n is 1, 2, 3, 4, 5, 6, 7, 8, 9, 10, 11, 12, 13, 14, 15, 16, 17, 18, 19 or 20. In some aspects, the IP5 substituted compound is a sodium salt. In some aspects, the IP5 substituted compound is a tetrasodium salt, pentasodium salt, hexasodium salt, heptasodium salt, octasodium salt, nonasodium salt or decasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. In some aspects, the IP5 substituted compound is an octasodium salt. In some aspects, n is 5, alkyl is CH₂ and X is —H (Compound 1). In some aspects, n is 3, alkyl is CH₂ and X is —H (Compound 24). In some aspects, n is 1, alkyl is CH₂ and X is —H (Compound 22). In some aspects, n is 5, alkyl is CH₂ and X is —OH (Compound 2). In some aspects, n is 10, alkyl is CH₂ and X is —OH (Compound 21). In some aspects, n is 5, alkyl is CH₂ and X is —OMe (Compound 3). In some aspects, n is 7, alkyl is CH₂ and X is —OMe (Compound 23). In some aspects, n is 5, alkyl is CH₂ and X is —COOH (Compound 4). In some aspects, n is 10, alkyl is CH₂ and X is —COOH (Compound 20). In some aspects, n is 3, alkyl is CH₂ and X is —CH(CH₃)₂ (Compound 12). In some aspects, n is 5, alkyl is CH₂ and X is —CF₃ (Compound 13). In some aspects, n is 5, alkyl is CH₂ and X is —NHCOMe (Compound 19). In some aspects, n is 2, alkyl is CH₂ and X is cyclopentane (Compound 16). In some aspects, n is 2, alkyl is CH₂ and X is cyclopropane (Compound 17). In some aspects, n is 5, alkyl is CH₂ and X is pyrazole (Compound 18). In some aspects, n is 5, alkyl is CH₂ and X is —CONH₂ (Compound 33).

In some aspects, n is an integer between 3 and 30, alkyl is CH₂ and X is an amine group. See FIG. 1A; Wang, 2014, supra. In some aspects, n is an integer between 3 and 20. In some aspects, n is an integer between 3 and 10. In some aspects, n is 3, 4, 5, 6, 7, 8, 9, 10, 11, 12, 13, 14, 15, 16, 17, 18, 19 or 20. In some aspects, the IP5 substituted compound is a sodium salt. In some aspects, the IP5 substituted compound is a tetrasodium salt, pentasodium salt, hexasodium salt, heptasodium salt, octasodium salt, nonasodium salt or decasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. In some aspects, the IP5 substituted compound is an octasodium salt. In some aspects, n is 3, alkyl is CH₂ and X is —NH₂ (Compound 27). In some aspects, n is 6, alkyl is CH₂ and X is —NH₂ (Compound 28).

In some aspects, an IP5 substituted compound of the present invention comprises or consists of a compound of the following formula:

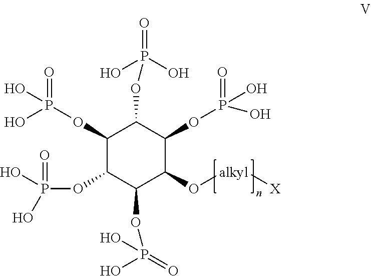

wherein n is an integer between 1 and 30, alkyl is CH₂ and X is —H, —OH, —OMe, amine group, pyrazole, triazole, —COOH, CONRR′, —NHCOR, —NHCOOR, —OCONR, —NHSO₂R, —NHCONRR′, —CF₃, cyclopropane, cyclopentane, pyrazole or alkynyl group. See FIG. 2A. In some aspects, n is an integer between 1 and 20. In some aspects, n is an integer between 1 and 10. In some aspects, n is 1, 2, 3, 4, 5, 6, 7, 8, 9, 10, 11, 12, 13, 14, 15, 16, 17, 18, 19, 21, 22, 23, 24, 25, 26, 27, 28, 29 or 30. In some aspects, the IP5 substituted compound is a sodium salt. In some aspects, the IP5 substituted compound is a tetrasodium salt, pentasodium salt, hexasodium salt, heptasodium salt, octasodium salt, nonasodium salt or decasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. In some aspects, the IP5 substituted compound is an octasodium salt. In some aspects, n is 5, alkyl is CH₂ and X is —H (Compound 6). In some aspects, n is 3, alkyl is CH₂ and X is —H (Compound 34). In some aspects, n is 5, alkyl is CH₂ and X is —OH (Compound 40). In some aspects, n is 5, alkyl is CH₂ and X is —OMe (Compound 7). In some aspects, n is 9, alkyl is CH₂ and X is —OMe (Compound 8). In some aspects, n is 19, alkyl is CH₂ and X is —OMe (Compound 9). In some aspects, n is 29, alkyl is CH₂ and X is —OMe (Compound 10). In some aspects, n is 5, alkyl is CH₂ and X is —NHCOMe (Compound 35). In some aspects, n is 5, alkyl is CH₂ and X is —CF₃ (Compound 36). In some aspects, n is 5, alkyl is CH₂ and X is —CONH₂ (Compound 37). In some aspects, n is 2, alkyl is CH₂ and X is cyclopentane (Compound 38). In some aspects, n is 2, alkyl is CH₂ and X is cyclopropane (Compound 39). In some aspects, n is 1, alkyl is CH₂ and X is C≡CH (Compound 43).

In some aspects, an IP5 substituted compound of the present invention comprises or consists of a compound of the following formula:

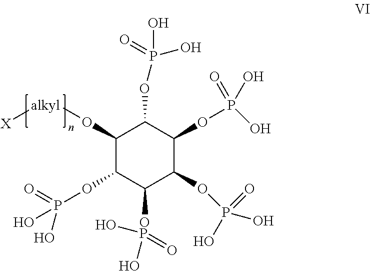

wherein n is an integer between 1 and 30, alkyl is CH₂ and X is —H, —OH, —OMe, amine group, pyrazole, triazole, —COOH, CONRR′, —NHCOR, —NHCOOR, —OCONR, —NHSO₂R, —NHCONRR′, —CF₃, cyclopropane, cyclopentane or pyrazole. See FIG. 3A. In some aspects, n is an integer between 1 and 20. In some aspects, n is an integer between 1 and 10. In some aspects, n is 1, 2, 3, 4, 5, 6, 7, 8, 9, 10, 11, 12, 13, 14, 15, 16, 17, 18, 19 or 20. In some aspects, the IP5 substituted compound is a sodium salt. In some aspects, the IP5 substituted compound is a tetrasodium salt, pentasodium salt, hexasodium salt, heptasodium salt, octasodium salt, nonasodium salt or decasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. In some aspects, the IP5 substituted compound is an octasodium salt. In some aspects, n is 3, alkyl is CH₂ and X is —H (Compound 29). In some aspects, n is 9, alkyl is CH₂ and X is —OMe (Compound 31). In some aspects, n is 5, alkyl is CH₂ and X is —OMe (Compound 44). In some aspects, n is 5, alkyl is CH₂ and X is —CF₃ (Compound 45). In some aspects, n is 2, alkyl is CH₂ and X is cyclopropane (Compound 30).

In some aspects, an IP5 substituted compound of the present invention comprises or consists of a compound of the following formula:

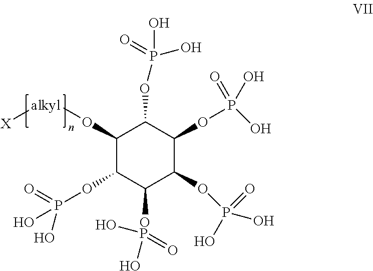

wherein n is an integer between 1 and 30, alkyl is CH₂ and X is —H, —OH, —OMe, amine group, pyrazole, triazole, —COOH, CONRR′, —NHCOR, —NHCOOR, —OCONR, —NHSO₂R, —NHCONRR′, —CF₃, cyclopropane, cyclopentane or pyrazole. See FIG. 4. In some aspects, n is an integer between 1 and 20. In some aspects, n is an integer between 1 and 10. In some aspects, n is 1, 2, 3, 4, 5, 6, 7, 8, 9, 10, 11, 12, 13, 14, 15, 16, 17, 18, 19 or 20. In some aspects, the IP5 substituted compound is a sodium salt. In some aspects, the IP5 substituted compound is a tetrasodium salt, pentasodium salt, hexasodium salt, heptasodium salt, octasodium salt, nonasodium salt or decasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. In some aspects, the IP5 substituted compound is an octasodium salt. In some aspects, n is 3, alkyl is CH₂ and X is —H (Compound 32). In some aspects, n is 9, alkyl is CH₂ and X is —OMe (Compound 47). In some aspects, n is 5, alkyl is CH₂ and X is —NHCOMe (Compound 49). In some aspects, n is 5, alkyl is CH₂ and X is —CF₃ (Compound 50). In some aspects, n is 2, alkyl is CH₂ and X is cyclopropane (Compound 48). In some aspects, n is 2, alkyl is CH₂ and X is cyclopentane (Compound 51).

In some aspects, an IP5 substituted compound of the present invention comprises or consists of a compound of the following formula:

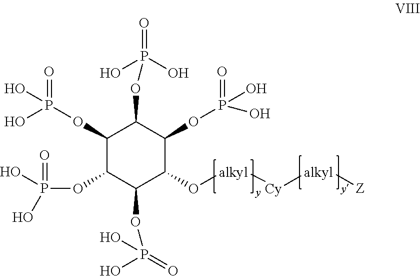

wherein y and y′ is an integer between 0 and 10, alkyl is CH₂, Cy is selected from the group consisting of 1,3-substituted phenyl, 1,4-substituted phenyl, piperazine, triazole-1, and triazole-2, and Z is selected from the group consisting of —CH₃, —OMe, —CF₃, COCH₃, and —COOH₃. See FIG. 1B. In some aspects, the IP5 substituted compound is a sodium salt. In some aspects, the IP5 substituted compound is a tetrasodium salt, pentasodium salt, hexasodium salt, heptasodium salt, octasodium salt, nonasodium salt or decasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. In some aspects, the IP5 substituted compound an octasodium salt or nonasodium salt. In some aspects, y or y′ is 0, 1, 2, 3, 4, 5, 6, 7, 8, 9 or 10. In some aspects, y is 3, y′ is 0, alkyl is CH₂, Cy is 1,4-substituted phenyl, and Z is —CH₃(Compound 11). In some aspects, y is 3, y′ is 0, alkyl is CH₂, Cy is 1,4-substituted phenyl, and Z is —OMe (Compound 14). In some aspects, y is 3, y′ is 0, alkyl is CH₂, Cy is 1,3-substituted phenyl, and Z is —CF₃ (Compound 15). In some aspects, y is 2, y′ is 0, alkyl is CH₂, Cy is piperazine, and Z is —COCH₃ (Compound 5). In some aspects, y is 3, y′ is 2, alkyl is CH₂, Cy is triazole-1, and Z is —COOH (Compound 25). In some aspects, y is 6, y′ is 0, alkyl is CH₂, Cy is triazole-1, and Z is —COOH (Compound 26).

In some aspects, an IP5 substituted compound of the present invention comprises or consists of a compound of the following formula:

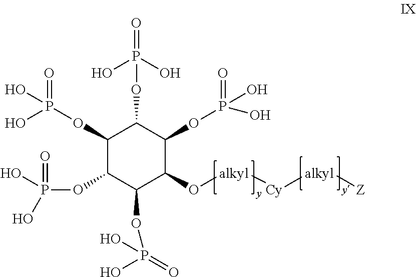

wherein y and y′ is an integer between 0 and 10, alkyl is CH₂, Cy is selected from the group consisting of 1,3-substituted phenyl, 1,4-substituted phenyl, piperazine, triazole-1, and triazole-2, and Z is selected from the group consisting of —CH₃, —OMe, —CF₃, COCH₃, and —COOH₃. See FIG. 2B. In some aspects, the IP5 substituted compound is a sodium salt. In some aspects, the IP5 substituted compound is a tetrasodium salt, pentasodium salt, hexasodium salt, heptasodium salt, octasodium salt, nonasodium salt or decasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. In some aspects, the IP5 substituted compound an octasodium salt or nonasodium salt. In some aspects, y or y′ is 0, 1, 2, 3, 4, 5, 6, 7, 8, 9 or 10. In some aspects, y is 5, y′ is 1, alkyl is CH₂, Cy is triazole-1, and Z is —OMe (Compound 41). In some aspects, y is 1, y′ is 2, alkyl is CH₂, Cy is triazole-2, and Z is —COOH (Compound 46).

In some aspects, an IP5 substituted compound of the present invention comprises or consists of a compound of the following formula:

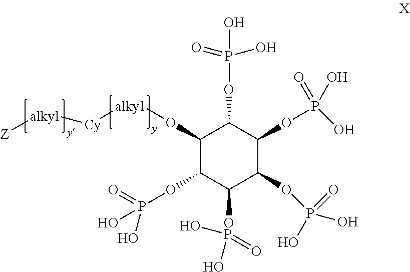

wherein y and y′ is an integer between 0 and 10, alkyl is CH₂, Cy is selected from the group consisting of 1,3-substituted phenyl, 1,4-substituted phenyl, piperazine, triazole-1, and triazole-2, and Z is selected from the group consisting of —CH₃, —OMe, —CF₃, COCH₃, and —COOH₃. See FIG. 3. In some aspects, the IP5 substituted compound is a sodium salt. In some aspects, the IP5 substituted compound is a tetrasodium salt, pentasodium salt, hexasodium salt, heptasodium salt, octasodium salt, nonasodium salt or decasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. In some aspects, the IP5 substituted compound an octasodium salt or nonasodium salt. In some aspects, y or y′ is 0, 1, 2, 3, 4, 5, 6, 7, 8, 9 or 10. In some aspects, y is 5, y′ is 1, alkyl is CH₂, Cy is triazole-1, and Z is —OMe (Compound 42).

In some aspects, an IP5 substituted compound of the present invention comprises or consists of a compound of the following formula:

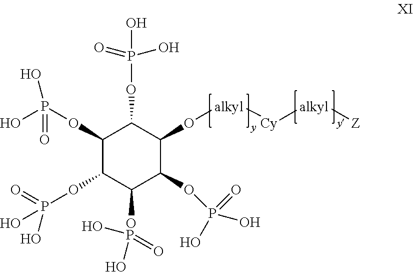

wherein y and y′ is an integer between 0 and 10, alkyl is CH₂, Cy is selected from the group consisting of 1,3-substituted phenyl, 1,4-substituted phenyl, piperazine, triazole-1, and triazole-2, and Z is selected from the group consisting of —CH₃, —OMe, —CF₃, COCH₃, and —COOH₃. See FIG. 4. In some aspects, the IP5 substituted compound is a sodium salt. In some aspects, the IP5 substituted compound is a tetrasodium salt, pentasodium salt, hexasodium salt, heptasodium salt, octasodium salt, nonasodium salt or decasodium salt. In some aspects, the IP5 substituted compound is a tetrasodium salt, pentasodium salt, hexasodium salt, heptasodium salt or octasodium salt. In some aspects, the IP5 substituted compound is a hexasodium salt, octasodium salt, nonasodium salt or decasodium salt. In some aspects, the IP5 substituted compound an octasodium salt or nonasodium salt. In some aspects, y or y′ is 0, 1, 2, 3, 4, 5, 6, 7, 8, 9 or 10. In some aspects, y is 5, y′ is 1, alkyl is CH₂, Cy is triazole-1, and Z is —OMe (Compound 52). In some aspects, y is 1, y′ is 2, alkyl is CH₂, Cy is triazole-2, and Z is —COOH (Compound 53).

TABLE 1
Intermediates

| No. | Formula | Intermediate |
|---|---|---|
| 01 | II_A-1 | rac-2-O-tert-Butyldimethylsilyl-1,3,5-O-methylidyne-4-O-pentyl-myo-inositol |
| | | (II_A-1) |
| 02 | II_A-2 | rac-4-O-(5-(Benzyloxy)pentyl)-2-O-tert-butyldimethylsilyl-1,3,5-O-methylidyne- |
| | | myo-inositol (II_A-2) |
| 03 | II_A-3 | rac-2-O-tert-Butyldimethylsilyl-4-O-(5-methoxypentyl)-1,3,5-O-methylidyne- |
| | | myo-inositol |
| 04 | II_A-4 | rac-2-O-tert-Butyldimethylsilyl-4-O-(5-ethoxycarbonylpentyl)-1,3,5-O- |
| | | methylidyne-myo-inositol |
| 05 | II_A-5 | rac-4-O-(2-(4-Acetylpiperazin-1-yl)ethyl)-2-O-tert-butyldimethylsilyl-1,3,5-O- |
| | | methylidyne-myo-inositol |
| 06 | II_B-1 | 4,6-bis-O-(4-Methoxybenzyl)-1,3,5-O-methylidyne-2-O-pentyl-myo-inositol |
| 07 | II_B-2 | 4,6-bis-O-(4-Methoxybenzyl)-2-O-(5-methoxypentyl)-1,3,5-O-methylidyne-myo- |
| | | inositol |
| 08 | II_B-3 | 4,6-bis-O-(4-Methoxybenzyl)-2-O-(9-methoxynonyl)-1,3,5-O-methylidyne-myo- |
| | | inositol |
| 09 | II_B′-1 | 4,6-di-O-Benzyl-2-O-(19-methoxynonadec-10-yn-1-yl)-1,3,5-O-methylidyne- |
| | | myo-inositol |
| 10 | II_B′-2 | 4,6-di-O-Benzyl-2-O-(29-methoxynonacosa-9,20-diyn-1-yl)-1,3,5-O- |
| | | methylidyne-myo-inositol |
| 11 | II_A-6 | rac-2-O-tert-Butyldimethylsilyl-1,3,5-O-methylidyne4-O-(3-(p-tolyl)propyl)- |
| | | myo-inositol |
| 12 | II_A-7 | rac-2-O-tert-Butyldimethylsilyl-1,3,5-O-methylidyne-4-O-(4-methylpentyl)-myo- |
| | | inositol |
| 13 | II_A-8 | rac-2-O-tert-Butyldimethylsilyl-1,3,5-O-methylidyne-4-O-(6,6,6-trifluorohexyl)- |
| | | myo-inositol |
| 14 | II_A-9 | rac-2-O-tert-Butyldimethylsilyl-4-O-(3-(4-methoxyphenyl)propyl)-1,3,5-O- |
| | | methylidyne-myo-inositol |
| 15 | II_A-10 | rac-2-O-tert-Butyldimethylsilyl-1,3,5-O-methylidyne-4-O-(3-(3- |
| | | (trifluoromethyl)phenyl)propyl)-myo-inositol |
| 16 | II_A-11 | rac-2-O-tert-Butyldimethylsilyl-4-O-(2-cyclopentylethyl)-1,3,5-O-methylidyne- |
| | | myo-inositol |
| 17 | II_A-12 | rac-2-O-tert-Butyldimethylsilyl-4-O-(2-cyclopropylethyl)-1,3,5-O-methylidyne- |
| | | myo-inositol |
| 18 | II_A-13 | rac-2-O-tert-Butyldimethylsilyl-1,3,5-O-methylidyne-4-O-(5-(1H-pyrazol-1- |
| | | yl)pentyl)--myo-inositol |
| 19 | II_A-14 | rac-4-O-(5-Acetamidopentyl)-2-O-tert-butyldimethylsilyl-1,3,5-O-methylidyne- |
| | | myo-inositol |
| 20 | II_A-15 | rac-2-O-tert-Butyldimethylsilyl-4-O-(10-ethoxycarbonyldecyl)-1,3,5-O- |
| | | methylidyne-myo-inositol |
| 21 | II_A-16 | rac-4-O-(10-(Benzyloxy)decyl)-2-O-tert-butyldimethylsilyl-1,3,5-O- |
| | | methylidyne-myo-inositol |
| 22 | II_A-17 | rac-2-O-tert-Butyldimethylsilyl-4-O-methyl-1,3,5-O-methylidyne-myo-inositol |
| 23 | II_A-18 | rac-2-O-tert-Butyldimethylsilyl-4-O-(7-methoxyheptyl)-1,3,5-O-methylidyne- |
| | | myo-inositol |
| 24 | II_A-19 | rac-2-O-tert-Butyldimethylsilyl-1,3,5-O-methylidyne-4-O-propyl-myo-inositol |
| 25 | II_C-1 | 1,2,3,4,6-penta-O-Benzyl-5-O-propargyl-myo-inositol |
| 26 | II_C-2 | 1,2,3,4,6-penta-O-Benzyl-5-O-(2-cyclopropylethyl)-myo-inositol |
| 27 | II_C-3 | 1,2,3,4,6-penta-O-bBnzyl-5-O-(9-methoxynonyl)-myo-inositol |
| 28 | II_D-1 | rac-2,4,6-tri-O-Benzyl-3,5-O-ethylidene-1-O-propargyl-myo-inositol |
| 29 | II_A-1 | rac-4-O-Pentyl-myo-inositol |
| 30 | II_A-2 | rac-4-O-(5-(Benzyloxy)pentyl)-myo-inositol |
| 31 | II_A-3 | rac-4-O-(5-Methoxypentyl)-myo-inositol |
| 32 | II_A-4 | rac-4-O-(5-Methoxycarbonylpentyl)-myo-inositol |
| 33 | II_A-5 | rac-4-O-(2-(4-Acetylpiperazin-1-yl)ethyl)-myo-inositol |
| 34 | II_B-1 | 2-O-Pentyl-myo-inositol |
| 35 | II_B-2 | 2-O-(5-Methoxypentyl)-myo-inositol |
| 36 | II_B-3 | 2-O-(9-Methoxynonyl)-myo-inositol |
| 37 | II_B-4 | 2-O-(19-Methoxynonadecyl)-myo-inositol |
| 38 | II_B-5 | 2-O-(29-Methoxynonacosyl)-myo-inositol |
| 29 | II_A-6 | rac-4-O-(3-(p-Tolyl)propyl)-myo-inositol |
| 40 | II_A-7 | rac-4-O-(4-Methylpentyl)-myo-inositol |
| 41 | II_A-8 | rac-4-O-(6,6,6-Trifluorohexyl)-myo-inositol |
| 42 | II_A-9 | rac-4-O-(3-(4-Methoxyphenyl)propyl)-myo-inositol |
| 43 | II_A-10 | rac-4-O-(3-(3-(Trifluoromethyl)phenyl)propyl)-myo-inositol |
| 44 | II_A-11 | rac-4-O-(2-Cyclopentylethyl)-myo-inositol |
| 45 | II_A-12 | rac-4-O-(2-Cyclopropylethyl)-myo-inositol |
| 46 | II_A-13 | rac-4-O-(5-(1H-Pyrazol-1-yl)pentyl)-myo-inositol |
| 47 | II_A-14 | rac-4-O-(5-Acetamidopentyl)-myo-inositol |
| 48 | II_A-15 | rac-4-O-(10-Methoxycarbonyldecyl)-myo-inositol |
| 49 | II_A-16 | rac-4-O-(10-(Benzyloxy)decyl)-myo-inositol |
| 50 | II_A-17 | rac-4-O-Methyl-myo-inositol |
| 51 | II_A-18 | rac-4-O-(7-Methoxyheptyl)-myo-inositol |
| 52 | II_A-19 | rac-4-O-Propyl-myo-inositol |
| 53 | II_A-20 | rac-4-O-(3-(4-(2-(Benzyloxycarbonyl)ethyl)-1H-1,2,3-triazol-1-yl)propyl)-myo- |
| | | inositol |
| 54 | II_A-21 | rac-4-O-(6-(4-(Methoxycarbonyl)-1H-1,2,3-triazol-1-yl)hexyl)-myo-inositol |
| 55 | II_C-1 | 5-O-Propyl-myo-inositol |
| 56 | II_C-2 | 5-O-(2-Cyclopropylethyl)-myo-inositol |
| 57 | II_C-3 | 5-O-(9-Methoxynonyl)-myo-inositol |
| 58 | II_D-1 | rac-1-O-Propyl-myo-inositol |
| 59 | IV_A-1 | rac-1,2,3,5,6-pentakis-O-(3-Oxido-1,5-dihydrobenzo[e][1,3,2]dioxaphosphepin- |
| | | 3-yl)-4-O-pentyl-myo-inositol |
| 60 | IV_A-2 | rac-4-O-(5-(Benzyloxy)pentyl)-1,2,3,5,6-pentakis-O-(3-oxido-1,5- |
| | | dihydrobenzo[e][1,3,2]dioxaphosphepin-3-yl)-myo-inositol |
| 61 | IV_A-3 | rac-4-O-(5-Methoxypentyl)-1,2,3,5,6-pentakis-O-(3-oxido-1,5- |
| | | dihydrobenzo[e][1,3,2]dioxaphosphepin-3-yl)-myo-inositol |
| 62 | IV_A-4 | rac-4-O-(5-Methoxycarbonylpentyl)-1,2,3,5,6-pentakis-O-(3-oxido-1,5- |
| | | dihydrobenzo[e][1,3,2]dioxaphosphepin-3-yl)myo-inositol |
| 63 | IV_A-5 | rac-4-O-(2-(4-Acetylpiperazin-1-yl)ethyl)-1,2,3,5,6-pentakis-O-(3-oxido-1,5- |
| | | dihydrobenzo[e][1,3,2]dioxaphosphepin-3-yl)-myo-inositol |
| 64 | IV_B-1 | 1,3,4,5,6-pentakis-O-(3-Oxido-1,5-dihydrobenzo[e][1,3,2]dioxaphosphepin-3-yl)- |
| | | 2-O-pentyl-myo-inositol |
| 65 | IV_B-2 | 2-O-(5-Methoxypentyl)-1,3,4,5,6-pentakis-O-(3-oxido-1,5- |
| | | dihydrobenzo[e][1,3,2]dioxaphosphepin-3-yl)-myo-inositol |
| 66 | IV_B-3 | 2-O-(9-Methoxynonyl)-1,3,4,5,6-pentakis-O-(3-oxido-1,5- |
| | | dihydrobenzo[e][1,3,2]dioxaphosphepin-3-yl)-myo-inositol |
| 67 | IV_B-4 | 2-O-(19-Methoxynonadecyl)-1,3,4,5,6-pentakis-O-(3-oxido-1,5- |
| | | dihydrobenzo[e][1,3,2]dioxaphosphepin-3-yl)-myo-inositol |
| 68 | IV_B-5 | 2-O-(29-Methoxynonacosyl)-1,3,4,5,6-pentakis-O-(3-oxido-1,5- |
| | | dihydrobenzo[e][1,3,2]dioxaphosphepin-3-yl)-myo-inositol |
| 69 | IV_A-6 | rac-1,2,3,5,6-pentakis-O-(3-oxido-1,5-dihydrobenzo[e][1,3,2]dioxaphosphepin-3- |
| | | yl)-4-O-(3-(p-Tolyl)propyl)-myo-inositol |
| 70 | IV_A-7 | rac-4-O-(4-Methylpentyl)-1,2,3,5,6-pentakis-O-(3-oxido-1,5- |
| | | dihydrobenzo[e][1,3,2]dioxaphosphepin-3-yl)-myo-inositol |
| 71 | IV_A-8 | rac-1,2,3,5,6-pentakis-O-(3-Oxido-1,5-dihydrobenzo[e][1,3,2]dioxaphosphepin- |
| | | 3-yl)-4-O-(6,6,6-Trifluorohexyl)-myo-inositol |
| 72 | IV_A-9 | rac-4-O-(3-(4-Methoxyphenyl)propyl)-1,2,3,5,6-pentakis-O-(3-oxido-1,5- |
| | | dihydrobenzo[e][1,3,2]dioxaphosphepin-3-yl)-myo-inositol |
| 73 | IV_A-10 | rac-1,2,3,5,6-pentakis-O-(3-Oxido-1,5-dihydrobenzo[e][1,3,2]dioxaphosphepin- |
| | | 3-yl)-4-O-(3-(3-(Trifluoromethyl)phenyl)propyl)-myo-inositol |
| 74 | IV_A-11 | rac-4-O-(2-Cyclopentylethyl)-1,2,3,5,6-pentakis-O-(3-oxido-1,5- |
| | | dihydrobenzo[e][1,3,2]dioxaphosphepin-3-yl)-myo-inositol |
| 75 | IV_A-12 | rac-4-O-(2-Cyclopropylethyl)-1,2,3,5,6-pentakis-O-(3-oxido-1,5- |
| | | dihydrobenzo[e][1,3,2]dioxaphosphepin-3-yl)-myo-inositol |
| 76 | IV_A-13 | rac-1,2,3,5,6-pentakis-O-(3-Oxido-1,5-dihydrobenzo[e][1,3,2]dioxaphosphepin- |
| | | 3-yl)-4-O-(5-(1H-Pyrazol-1-yl)pentyl)-myo-inositol |
| 77 | IV_A-14 | rac-4-O-(5-Acetamidopentyl)-1,2,3,5,6-pentakis-O-(3-oxido-1,5- |
| | | dihydrobenzo[e][1,3,2]dioxaphosphepin-3-yl)-myo-inositol |
| 78 | IV_A-15 | rac-4-O-(10-Methoxycarbonyldecyl)-1,2,3,5,6-pentakis-O-(3-oxido-1,5- |
| | | dihydrobenzo[e][1,3,2]dioxaphosphepin-3-yl)-myo-inositol |
| 79 | IV_A-16 | rac-4-O-(10-(Benzyloxy)decyl)-1,2,3,5,6-pentakis-O-(3-oxido-1,5- |
| | | dihydrobenzo[e][1,3,2]dioxaphosphepin-3-yl)-myo-inositol |
| 80 | IV_A-17 | rac-4-O-Methyl-1,2,3,5,6-pentakis-O-(3-oxido-1,5- |
| | | dihydrobenzo[e][1,3,2]dioxaphosphepin-3-yl)-myo-inositol |
| 81 | IV_A-18 | rac-4-O-(7-Methoxyheptyl)-1,2,3,5,6-pentakis-O-(3-oxido-1,5- |
| | | dihydrobenzo[e][1,3,2]dioxaphosphepin-3-yl)-myo-inositol |
| 82 | IV_A-19 | rac-1,2,3,5,6-pentakis-O-(3-Oxido-1,5-dihydrobenzo[e][1,3,2]dioxaphosphepin- |
| | | 3-yl)-4-O-Propyl-myo-inositol |
| 83 | IV_A-20 | rac-4-O-(3-(4-(2-(Benzyloxycarbonyl)ethyl)-1H-1,2,3-triazol-1-yl)propyl)- |
| | | 1,2,3,5,6-pentakis-O-(3-oxido-1,5-dihydrobenzo[e][1,3,2]dioxaphosphepin-3-yl)- |
| | | myo-inositol |
| 84 | IV_A-21 | rac-4-O-(6-(4-(Methoxycarbonyl)-1H-1,2,3-triazol-1-yl)hexyl)-1,2,3,5,6- |
| | | pentakis-O-(3-oxido-1,5-dihydrobenzo[e][1,3,2]dioxaphosphepin-3-yl)-myo- |
| | | inositol |
| 85 | IV_C-1 | 1,2,3,4,6-pentakis-O-(3-Oxido-1,5-dihydrobenzo[e][1,3,2]dioxaphosphepin-3-yl)- |
| | | 5-O-Propyl-myo-inositol |
| 86 | IV_C-2 | 5-O-(2-Cyclopropylethyl)-1,2,3,4,6-pentakis-O-(3-oxido-1,5- |
| | | dihydrobenzo[e][1,3,2]dioxaphosphepin-3-yl)-myo-inositol |
| 87 | IV_C-3 | 5-O-(9-Methoxynonyl)-1,2,3,4,6-pentakis-O-(3-oxido-1,5- |
| | | dihydrobenzo[e][1,3,2]dioxaphosphepin-3-yl)-myo-inositol |
| 88 | IV_D-1 | rac-2,3,4,5,6-pentakis-O-(3-Oxido-1,5-dihydrobenzo[e][1,3,2]dioxaphosphepin- |
| | | 3-yl)-1-O-propyl-myo-inositol |
| 89 | V_A-1 | rac-4-O-(3-Azidopropyl)-2-O-tert-butyldimethylsilyl-1,3,5-O-methylidyne-myo- |
| | | inositol |
| 90 | V_A-2 | rac-4-O-(6-Azidohexyl)-2-O-tert-butyldimethylsilyl-1,3,5-O-methylidyne-myo- |
| | | inositol |
| 91 | VI_A-1 | rac-4-O-(3-Azidopropyl)-myo-inositol |
| 92 | VI_A-2 | rac-4-O-(6-Azidohexyl)-myo-inositol |
| 93 | VII_A-1 | rac-4-O-(3-Azidopropyl)-1,2,3,5,6-pentakis-O-(3-oxido-1,5- |
| | | dihydrobenzo[e][1,3,2]dioxaphosphepin-3-yl)-myo-inositol |
| 94 | VII_A-2 | rac-4-O-(3-Azidopropyl)-1,2,3,5,6-pentakis-O-(3-oxido-1,5- |
| | | dihydrobenzo[e][1,3,2]dioxaphosphepin-3-yl)-myo-inositol |
| 95 | VII_B-1 | 2-O-(19-Methoxynonadecyl)-1,3,5-O-methylidyne-myo-inositol |
| 96 | VII_B-2 | 2-O-(29-Methoxynonacosyl)-1,3,5-O-methylidyne-myo-inositol |
| 97 | IX_D-1 | rac-2,4,6-tri-O-Benzyl-1-O-propyl-myo-inositol |
| 98 | II_A-20 | rac-2-O-tert-Butyldimethylsilyl-4-O-(5-carboxypentyl)-1,3,5-O-methylidyne- |
| | | myo-inositol |
| 99 | II_A-21 | rac-4-O-(6-Amino-6-oxohexyl)-2-O-tert-butyldimethylsilyl-1,3,5-O- |
| | | methylidyne-myo-inositol |
| 100 | II_B-4 | 4,6-bis-O-(4-Methoxybenzyl)-2-O-propargyl-myo-inositol |
| 101 | II_B-5 | 2-O-(5-(Benzyloxy)pentyl)-4,6-bis-O-(4-methoxybenzyl)-1,3,5-O-methylidyne- |
| | | myo-inositol |
| 102 | II_B-6 | 2-O-(5-Aminopentyl)-4,6-bis-O-(4-methoxybenzyl)-1,3,5-O-methylidyne-myo- |
| | | inositol |
| 103 | II_B-7 | 4,6-bis-O-(4-Methoxybenzyl)-2-O-(5-(4-(methoxymethyl)-1H-1,2,3-triazol-1- |
| | | yl)pentyl)-1,3,5-O-methylidyne-myo-inositol |
| 104 | II_B′-3 | 4,6-di-O-Benzyl-2-O-(6,6,6-trifluorohexyl)-O-1,3,5-methylidyne-myo-inositol |
| 105 | II_B′-4 | 2-O-(6-Amino-6-oxohexyl)-4,6-di-O-benzyl-1,3,5-O-methylidyne-myo-inositol |
| 106 | II_B′-5 | 4,6-di-O-Benzyl-2-O-(2-cyclopentylethyl)-1,3,5-O-methylidyne-myo-inositol |
| 107 | II_B′-6 | 4,6-di-O-Benzyl-2-O-(2-cyclopropylethyl)-1,3,5-O-methylidyne-myo-inositol |
| 108 | II_C-4 | 1,2,3,4,6-penta-O-Benzyl-5-O-(5-methoxypentyl)-myo-inositol |
| 109 | II_C-5 | 1,2,3,4,6-penta-O-Benzyl-5-O-(6,6,6-trifluorohexyl)-myo-inositol |
| 110 | II_C-6 | 1,2,3,4,6-penta-O-Benzyl-5-O-(5-(4-(methoxymethyl)-1,2,3-triazol-1-yl)pentyl)- |
| | | myo-inositol |
| 111 | II_D-2 | rac-2,4,6-tri-O-Benzyl-3,5-O-ethylidene-1-O-(9-methoxynonyl)-myo-inositol |
| 112 | II_D-3 | rac-2,4,6-tri-O-Benzyl-1-O-(2-cyclopropylethyl)-3,5-O-ethylidene-myo-inositol |
| 113 | II_D-4 | rac-2,4,6-tri-O-Benzyl-3,5-O-ethylidene-2-O-propargyl-myo-inositol |
| 114 | II_D-5 | rac-2,4,6-tri-O-Benzyl-3,5-O-ethylidene-1-O-(6,6,6-trifluorohexyl)-myo-inositol |
| 115 | II_D-6 | rac-2,4,6-tri-O-Benzyl-1-O-(2-cyclopentylethyl)-3,5-O-ethylidene-myo-inositol |
| 116 | II_A-22 | rac-4-O-(6-Amino-6-oxohexyl)-myo-inositol |
| 117 | II_B-7 | 2-O-(5-(Benzyloxy)pentyl)-myo-inositol |
| 118 | II_B-8 | 2-O-(6,6,6-Trifluorohexyl)-myo-inositol |
| 119 | III-B-9 | 2-O-(6-Amino-6-oxohexyl)-myo-inositol |
| 120 | II_B-10 | 2-O-(2-Cyclopentylethyl)-myo-inositol |
| 121 | II_B-11 | 2-O-(2-Cyclopropylethyl)-myo-inositol |
| 122 | II_B-12 | 2-O-(5-Aminopentyl)-myo-inositol |
| 123 | II_B-13 | 2-O-Propargyl-myo-inositol |
| 124 | II_B-14 | 2-O-(5-(4-(Methoxymethyl)-1H-1,2,3-triazol-1-yl)pentyl)-myo-inositol |
| 125 | II_B-15 | 2-O-(5-Acetamidopentyl)-myo-inositol |
| 126 | II_B-16 | 2-O-((1-(2-(Metoxycarbonyl)ethyl)-1H-1,2,3-triazol-4-yl)methyl)-myo-inositol |
| 127 | II_C-4 | 5-O-(5-Methoxypentyl)-myo-inositol |
| 128 | II_C-5 | 5-O-(6,6,6-Trifluorohexyl)-myo-inositol |
| 129 | II_C-6 | 5-O-(5-(4-(Methoxymethyl)-1H-1,2,3-triazol-1-yl)pentyl)-myo-inositol |
| 130 | II_D-2 | rac-1-O-(9-Methoxynonyl)-myo-inositol |
| 131 | II_D-3 | rac-1-O-(2-Cyclopropylethyl)-myo-inositol |
| 132 | II_D-5 | rac-1-O-(6,6,6-Trifluorohexyl)-myo-inositol |
| 133 | II_D-6 | rac-1-O-(2-Cyclopentylethyl)-myo-inositol |
| 134 | II_D-7 | rac-1-O-((1-(2-(Metoxycarbonyl)ethyl)-1H-1,2,3-triazol-4-yl)methyl)-myo- |
| | | inositol |
| 135 | II_D-8 | rac-1-O-(5-(4-(Methoxymethyl)-1H-1,2,3-triazol-1-yl)pentyl)-myo-inositol |
| 136 | IV_A-22 | rac-4-O-(6-Amino-6-oxohexyl)-1,2,3,5,6-pentakis-O-(3-oxido-1,5- |
| | | dihydrobenzo[e][1,3,2]dioxaphosphepin-3-yl)-myo-inositol |
| 137 | IV_B-6 | 1,3,4,5,6-pentakis-O-(3-Oxido-1,5-dihydrobenzo[e][1,3,2]dioxaphosphepin-3-yl)- |
| | | 2-O-propargyl-myo-inositol |
| 138 | IV_B-7 | 2-O-(5-(Benzyloxy)pentyl)-1,3,4,5,6-pentakis-O-(3-oxido-1,5- |
| | | dihydrobenzo[e][1,3,2]dioxaphosphepin-3-yl)-myo-inositol |
| 139 | IV_B-8 | 2-O-(6,6,6-Trifluorohexyl)-1,3,4,5,6-pentakis-O-(3-oxido-1,5- |
| | | dihydrobenzo[e][1,3,2]dioxaphosphepin-3-yl)-myo-inositol |
| 140 | IV_B-9 | 2-O-(6-Amino-6-oxohexyl)-1,3,4,5,6-pentakis-O-(3-oxido-1,5- |
| | | dihydrobenzo[e][1,3,2]dioxaphosphepin-3-yl)-myo-inositol |
| 141 | IV_B-10 | 2-O-(2-Cyclopentylethyl)-1,3,4,5,6-pentakis-O-(3-oxido-1,5- |
| | | dihydrobenzo[e][1,3,2]dioxaphosphepin-3-yl)-myo-inositol |
| 142 | IV_B-11 | 2-O-(2-Cyclopropylethyl)-1,3,4,5,6-pentakis-O-(3-oxido-1,5- |
| | | dihydrobenzo[e][1,3,2]dioxaphosphepin-3-yl)-myo-inositol |
| 143 | IV_B-12 | 2-O-(5-(4-(Methoxymethyl)-1H-1,2,3-triazol-1-yl)pentyl)-1,3,4,5,6-pentakis-O- |
| | | (3-oxido-1,5-dihydrobenzo[e][1,3,2]dioxaphosphepin-3-yl)-myo-inositol |
| 144 | IV_B-13 | 2-O-(5-Acetamidopentyl)-1,3,4,5,6-pentakis-O-(3-oxido-1,5- |
| | | dihydrobenzo[e][1,3,2]dioxaphosphepin-3-yl)-myo-inositol |
| 145 | IV_B-14 | 2-O-((1-(2-(Metoxycarbonyl)ethyl)-1H-1,2,3-triazol-4-yl)methyl)-1,3,4,5,6- |
| | | pentakis-O-(3-oxido-1,5-dihydrobenzo[e][1,3,2]dioxaphosphepin-3-yl)-myo- |
| | | inositol |
| 146 | IV_C-4 | 5-O-(5-Methoxypentyl)-1,2,3,4,6-pentakis-O-(3-oxido-1,5- |
| | | dihydrobenzo[e][1,3,2]dioxaphosphepin-3-yl)-myo-inositol |
| 147 | IV_C-5 | 5-O-(6,6,6-Trifluorohexyl)-1,2,3,4,6-pentakis-O-(3-oxido-1,5- |
| | | dihydrobenzo[e][1,3,2]dioxaphosphepin-3-yl)-myo-inositol |
| 148 | IV_C-6 | 5-O-(5-(4-(Methoxymethyl)-1H-1,2,3-triazol-1-yl)pentyl)-1,2,3,4,6-pentakis-O- |
| | | (3-oxido-1,5-dihydrobenzo[e][1,3,2]dioxaphosphepin-3-yl)-myo-inositol |
| 149 | IV_D-2 | rac-1-O-(9-Methoxynonyl)-2,3,4,5,6-pentakis-O-(3-oxido-1,5- |
| | | dihydrobenzo[e][1,3,2]dioxaphosphepin-3-yl)-myo-inositol |
| 150 | IV_D-3 | rac-1-O-(2-Cyclopropylethyl)-2,3,4,5,6-pentakis-O-(3-oxido-1,5- |
| | | dihydrobenzo[e][1,3,2]dioxaphosphepin-3-yl)-myo-inositol |
| 151 | IV_D-4 | rac-1-O-(5-Acetamidopentyl)-2,3,4,5,6-pentakis-O-(3-oxido-1,5- |
| | | dihydrobenzo[e][1,3,2]dioxaphosphepin-3-yl)-myo-inositol |
| 152 | IV_D-5 | rac-1-O-(6,6,6-Trifluorohexyl)-2,3,4,5,6-pentakis-O-(3-oxido-1,5- |
| | | dihydrobenzo[e][1,3,2]dioxaphosphepin-3-yl)-myo-inositol |
| 153 | IV_D-6 | rac-1-O-(2-Cyclopentylethyl)-2,3,4,5,6-pentakis-O-(3-oxido-1,5- |
| | | dihydrobenzo[e][1,3,2]dioxaphosphepin-3-yl)-myo-inositol |
| 154 | IV_D-7 | rac-1-O-((1-(2-(Metoxycarbonyl)ethyl)-1H-1,2,3-triazol-4-yl)methyl)-2,3,4,5,6- |
| | | pentakis-O-(3-oxido-1,5-dihydrobenzo[e][1,3,2]dioxaphosphepin-3-yl)-myo- |
| | | inositol |
| 155 | IV_D-8 | rac-1-O-(5-(4-(Methoxymethyl)-1H-1,2,3-triazol-1-yl)pentyl)-2,3,4,5,6-pentakis- |
| | | O-(3-oxido-1,5-dihydrobenzo[e][1,3,2]dioxaphosphepin-3-yl)-myo-inositol |
| 156 | V_B-1 | 2-O-(5-Azidopentyl)-4,6-bis-O-(4-methoxybenzyl)-1,3,5-O-methylidyne-myo- |
| | | inositol |
| 157 | V_C-1 | 5-O-(5-Azidopentyl)-1,2,3,4,6-penta-O-benzyl-myo-inositol |
| 158 | V_D-1 | rac-1-O-(5-Azidopentyl)-2,4,6-tri-O-benzyl-3,5-O-ethylidene-myo-inositol |
| 159 | VII_B′-1 | 4,6-di-O-Benzyl-2-O-(6,6,6-trifluorohexyl)-myo-inositol |
| 160 | VII_B′-2 | 2-O-(6-Amino-6-oxohexyl)-4,6-di-O-benzyl-myo-inositol |
| 161 | VII_B′-3 | 4,6-di-O-Benzyl-2-O-(2-cyclopentylethyl)-myo-inositol |
| 162 | VII_B′-4 | 4,6-di-O-Benzyl-2-O-(2-cyclopropylethyl)-myo-inositol |
| 163 | IX_D-2 | rac-2,4,6-tri-O-Benzyl-1-O-(9-methoxynonyl)-myo-inositol |
| 164 | IX_D-3 | rac-2,4,6-tri-O-Benzyl-1-O-(2-cyclopropylethyl)-myo-inositol |
| 165 | IX_D-4 | rac-1-O-(5-Aminopentyl)-2,4,6-tri-O-benzyl-myo-inositol |
| 166 | IX_D-5 | rac-2,4,6-tri-O-Benzyl-1-O-(6,6,6-trifluorohexyl)-myo-inositol |
| 167 | IX_D-6 | rac-2,4,6-tri-O-Benzyl-1-O-(2-cyclopentylethyl)-myo-inositol |
| 168 | IX_D-7 | rac-1-O-(5-Acetamidopentyl)-2,4,6-tri-O-benzyl-myo-inositol |
| 169 | IX_D-8 | rac-2,4,6-tri-O-Benzyl-1-O-(5-(4-(methoxymethyl)-1H-1,2,3-triazol-1-yl)pentyl)- |
| | | myo-inositol |
| 170 | IX_D-9 | rac-2,4,6-tri-O-Benzyl-2-O-propargyl-myo-inositol |
| 171 | IX_D-10 | rac-2,4,6-tri-O-Benzyl-1-O-((1-(2-(metoxycarbonyl)ethyl)-1H-1,2,3-triazol-4- |
| | | yl)methyl)-myo-inositol |
| 172 | X_D-1 | rac-1-O-(5-Azidopentyl)-2,4,6-tri-O-benzyl-myo-inositol |

IP5 substituted compounds of the present invention and intermediates for their synthesis can be synthesized by using the methods described herein, as well as other processes known in the field of the organic chemistry. In some aspects, the methods include, but are not limited to, the general procedures shown in the synthesis Schemes 1, 2, 3, 4, 5, 6, 7 and 8 described herein. Thus, in some aspects, the present invention provides a method to manufacture an IP5 substituted compound of the present invention comprising applying synthetic Scheme 1 disclosed below. In some aspects, the present invention provides a method to manufacture an IP5 substituted compound of the present invention comprising applying synthetic Scheme 2 disclosed below. In some aspects, the present invention provides a method to manufacture an IP5 substituted compound of the present invention comprising applying synthetic Scheme 3 disclosed below. In some aspects, the present invention provides a method to manufacture an IP5 substituted compound of the present invention comprising applying synthetic Scheme 4 disclosed below. In some aspects, the present invention provides a method to manufacture an IP5 substituted compound of the present invention comprising applying synthetic Scheme 5 disclosed below. In some aspects, the present invention provides a method to manufacture an IP5 substituted compound of the present invention comprising applying synthetic Scheme 6 disclosed below. In some aspects, the present invention provides a method to manufacture an IP5 substituted compound of the present invention comprising applying synthetic Scheme 7 disclosed below. In some aspects, the present invention provides a method to manufacture an IP5 substituted compound of the present invention comprising applying synthetic Scheme 8 disclosed below.

In some aspects, the present invention provides a method to manufacture an intermediate for the synthesis of an IP5 substituted compound of the present invention (e.g., a compound selected from the group consisting of Compound 1 to Compound 53) comprising applying any of synthetic Scheme 1, Scheme 2, Scheme 3, Scheme 4, Scheme, 5, Scheme 6, Scheme 7 or Scheme 8 disclosed below as applicable.

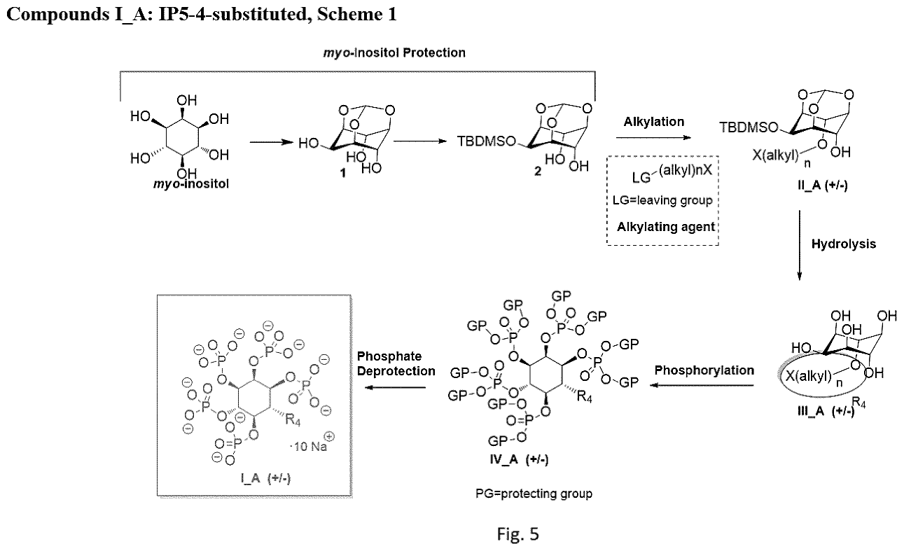

Scheme 1: In general, the IP5 substituted compounds of the present invention (i.e., Compounds I_A) can be obtained by deprotecting the intermediates of formula IV_A (Scheme 1). The “protective group” or GP, can be, without limitation, benzyl, levulinylbenzyl, tert-butyl, o,o′-xylenyl (by union of 2 PG in the same phosphate), 9-fluorenylmethyl, cyanoethyl and other suitable protective groups in each case. Intermediates of formula IV_A may be achieved by phosphorylation of an intermediate of formula III_A according to procedures described in the literature such as reaction with a phosphoroamidite derivative and subsequent oxidation. At the same time, intermediates of formula III_A can be obtained by hydrolysis in acid media of intermediates of formula II_A. Finally, intermediates of formula II_A may be obtained by alkylation of intermediate (2) with an alkylating agent. Preparation of (2) was previously described in the literature (Martin S, et al., J Org Chem 1994; 59(17): 4805-4820, Kardivel M, Org Biomol Chem 2008; 6(11):1966-1972). The “leaving Group” or LG, may be, without limitation, chloride, bromide, iodide, toluenesulfonyl (Ts) or methylsulfonyl (Ms). See FIG. 5.

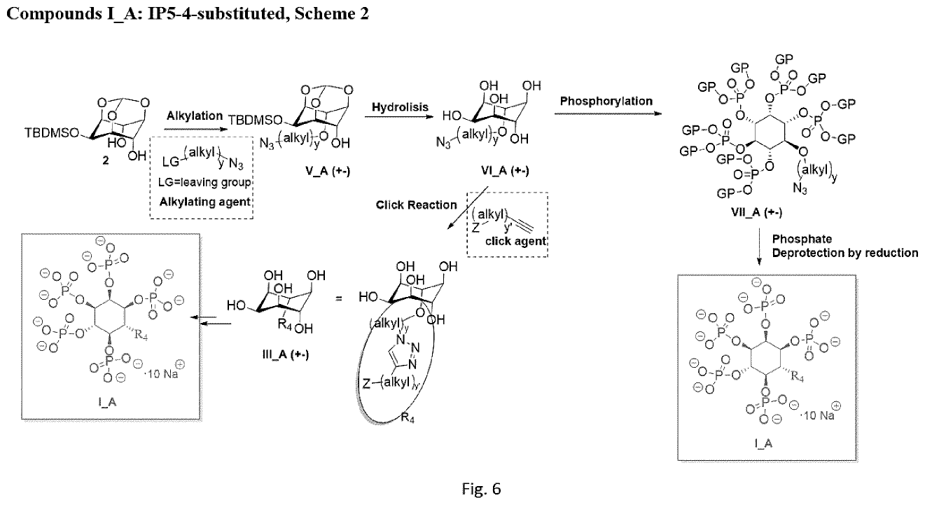

Scheme 2: As an alternative to Scheme 1, when R₄ or R₆ contain a substituted 1,2,3-triazole, compounds of formula I_A can be obtained by following the alternative route described in Scheme 2. In this way, intermediates of formula III_A can be obtained via a click reaction by using as starting materials an intermediate of formula VI_A and an alkynyl “click agent”. Intermediates VI_A can be achieved by alkylation and hydrolysis of intermediate 2 with an appropriate reagent. As another alternative, when R₄ or R₆ contain a terminal amine group, compounds of formula I_A can be obtained by phosphorylation of compound VI_A in order to obtain compounds VII-A, and by the subsequent deprotection/reduction of such compounds. See FIG. 6.

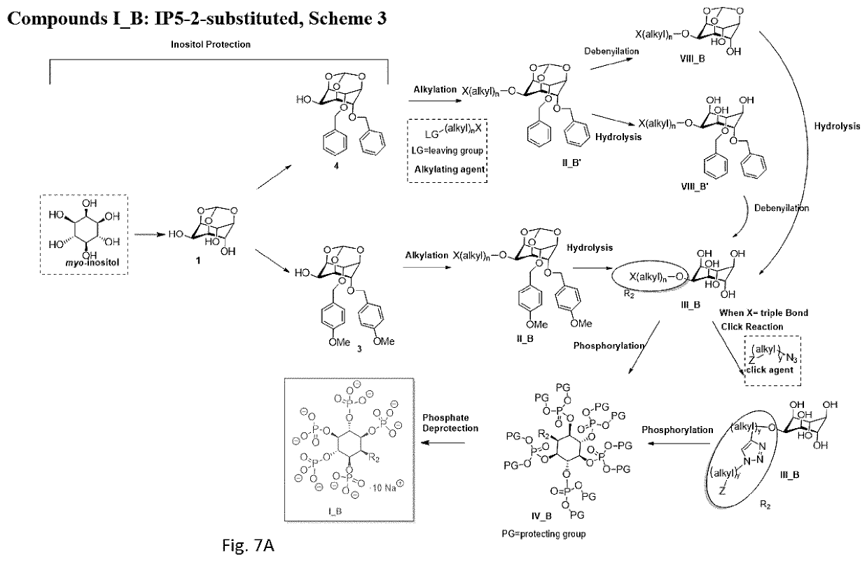

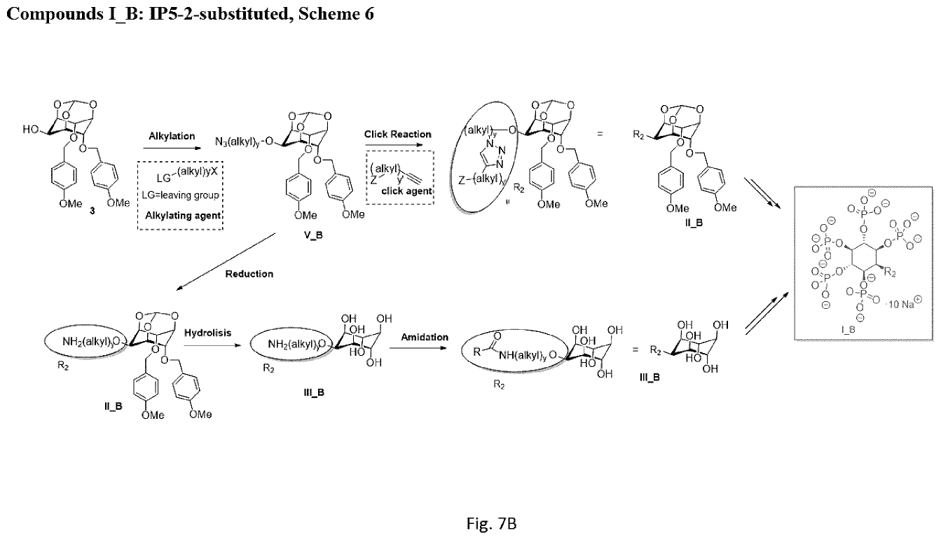

Scheme 3: Compounds I_B may be obtained using a similar synthetic route used for compounds I_A. Thus, compounds I_B may be obtained by deprotecting intermediates of formula IV_B (Scheme 3). Intermediates of formula IV_B may be attained by phosphorylating an intermediate of formula III_B. At the same time, intermediates of formula III_B can be obtained by the hydrolysis in acid media of intermediates of formula II_B or VIII_B. Intermediates of formula VIII_B, may be achieved by the debenzylation of intermediates II_B′. Finally, intermediates of formula II_B or B′ may be obtained by the alkylation of intermediates (3) or (4), respectively, with an alkylating agent. Preparations (3) and (4) have been described previously in the art (Aiba T, et al., Org Biomol Chem 2016; 14(28):6672-6675 and Chen W, et al., Eur J Med Chem 2015; 93:172-181). As an alternative, when R₂ contain a substituted 1,2,3-triazole, compounds of formula I_B can be obtained by following an alternative route. In this way, intermediates of formula III_B can be also obtained via a click reaction by using as starting materials alkynyl intermediate of formula III_B and an azide “click agent”. See FIG. 7A.

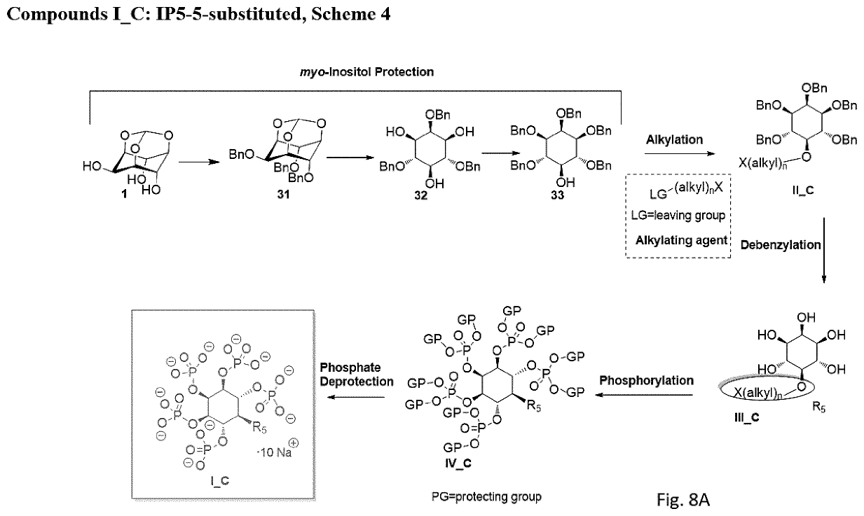

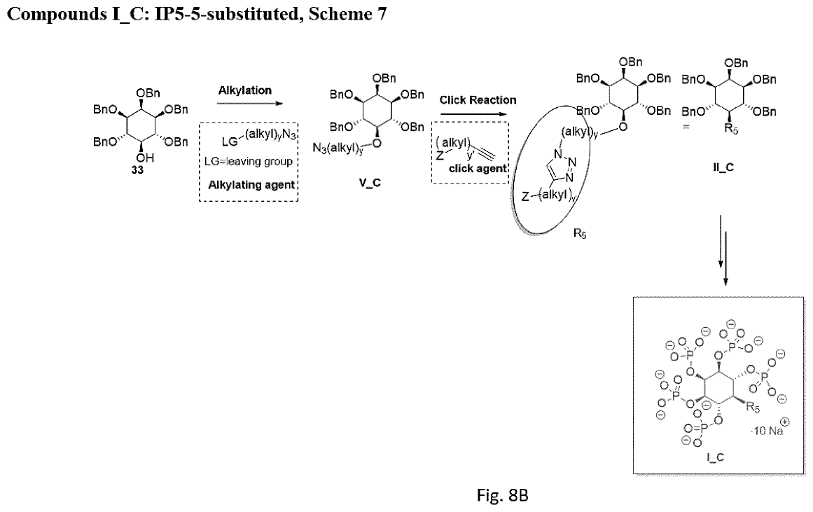

Scheme 4: Compounds I_C can be obtained by the deprotection of intermediates of formula IV_C (Scheme 4). Intermediates of formula IV_C may be achieved phosphorylating intermediates of formula IV_C. Intermediates of formula III_C can be obtained by the debenzylation of intermediates II_C. Finally, intermediates of formula II_C may be obtained by the alkylation of intermediate (33) with an appropriate alkylating agent. Preparation of (33) has been previously described in the art (Phenix C. et al., ChemBioChem 2008; 9(10):1591-1602). See FIG. 8A.

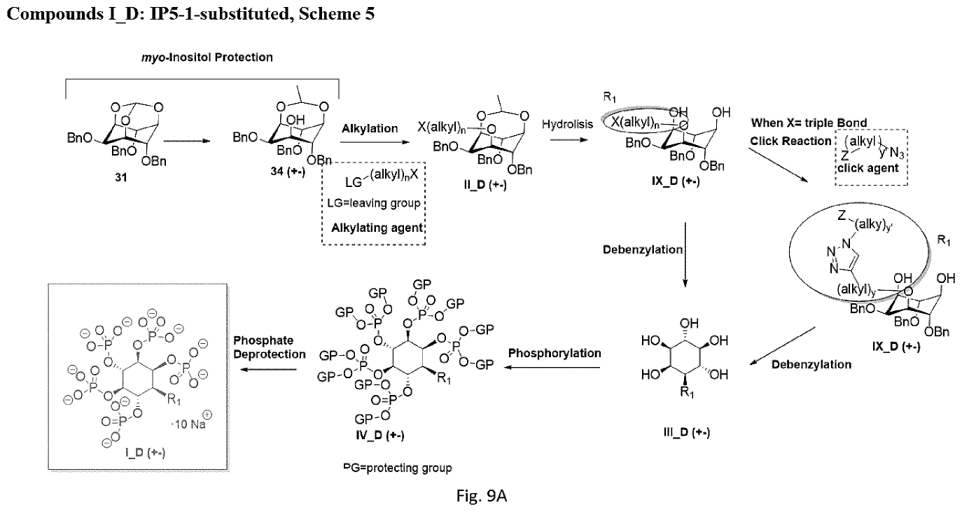

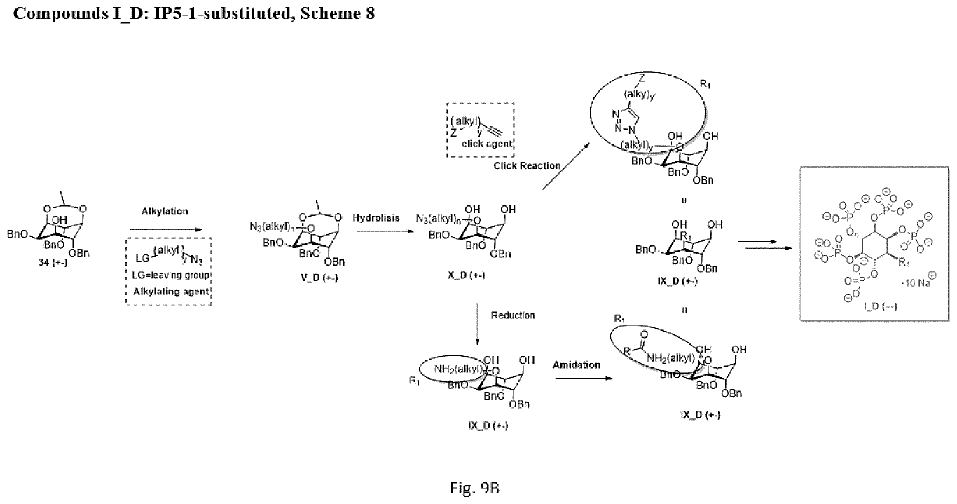

Scheme 5: Compounds I_D may be obtained by the deprotection of intermediates of formula IV_D (Scheme 5). Intermediates of formula IV_D may be achieved by phosphorylating intermediates of formula III_D. Intermediates of formula III_D can be obtained by the debenzylation of intermediates IX_D. Intermediates IX-D can be obtained by the hydrolysis of intermediates II_D. Finally, intermediates of formula II_D may be obtained by the alkylation of intermediate (34) with an appropriate alkylating agent. Preparation of racemate (34) has been in the art (Chen, 2015, supra). As an alternative, when R₂ contain a substituted 1,2,3-triazole, compounds of formula I_D can be obtained by following the alternative route. In this way, intermediates of formula IX_D can be also obtained via a click reaction by using as starting materials alkynyl intermediate of formula IX_D and an azide “click agent”. See FIG. 9A.

Scheme 6: As an alternative to Scheme 3, when R₂ contain a substituted 1,2,3-triazole or an acylamine, compounds of formula I_B can be obtained by following the alternative route described in Scheme 6. In this way, intermediates of formula III_B can be also obtained via a click reaction by using as starting materials an azide intermediate of formula V_B and an alkynyl “click agent”. Intermediate V_B can be achieved by alkylation of intermediate 3 with an appropriate reagent. As another alternative, when R₂ contains a terminal acylamine group, compounds of formula III_B can be obtained by amidation of the corresponding amino compound III_B. See FIG. 7B.

Scheme 7: As an alternative to Scheme 4, when R₅ contain a substituted 1,2,3-triazole, compounds of formula I_C can be obtained by following the alternative route described in Scheme 7. In this way, intermediates of formula II_C can be also obtained via a click reaction by using as starting materials an azide intermediate of formula V_C and an alkynyl “click agent”. Intermediate V_C can be achieved by alkylation of intermediate 33 with an appropriate reagent. See FIG. 8B.

Scheme 8: As an alternative to Scheme 5, when R₁ or R₃ contain a substituted 1,2,3-triazole, compounds of formula I_D can be obtained by following the alternative route described in Scheme 8. In this way, intermediates of formula IX_D can be also obtained via a click reaction by using as starting materials an alkinyl intermediate (where X is a triple bond) of formula V_C and an azide “click agent”. Intermediate V_C (where X is a triple bond) can be achieved by alkylation of intermediate 34 and subsequent hydrolysis of such alkenyl-protected compound II_D. As another alternative, When R₁ or R₃ contain an alkyl group, intermediates of formula III_D, with an alkyl as R₁, can be obtained via debenzilation of intermediate IX_D where X correspond to a triple bond. See FIG. 9A.

Representative IP5 substituted compounds of the present invention (e.g., a compound selected from the group consisting of Compound 1 to Compound 53) are presented herein and all of them are in the myo conformation. However, it is to be understood that any exemplary IP5 substituted compound of the present invention in the myo conformation is not limited to the representative conformation displayed. Thus, for example, Compounds 1 to 53 and the intermediates presented herein are in the myo conformation. In this conformation, the natural myo isomer has a structure in which five of the six hydroxyls (the first, third, fourth, fifth, and sixth) are equatorial, whereas the second hydroxyl group is axial.

The present invention also provides methods to manufacture a medicament for the treatment of pathological crystallization comprising using an intermediate compound selected from the group consisting of the compounds listed in Table 1. Also provided is a compound of formula I (e.g., selected from the group consisting of Compound 1 to Compound 53) for use as a medicament. Also provided is the use of a compound of formula I (e.g., selected from the group consisting of Compound 1 to Compound 53) for the manufacture of a medicament for the prevention or treatment of a disease related to pathological crystallization.

### III. Pharmaceutical Compositions

The present invention also provides pharmaceutical compositions for use in the methods for the prevention and/or treatment of diseases and conditions disclosed herein (e.g., pathological crystallizations), wherein the pharmaceutical composition comprises at least one IP5 substituted compound of the present invention (e.g., a compound selected from the group consisting of Compound 1 to Compound 53). In some aspects, the pharmaceutical composition comprises an IP5 substituted compound of the present invention (e.g., a compound selected from the group consisting of Compound 1 to Compound 53) alone or together with one or more pharmaceutically acceptable excipients or carriers.

The term “excipient” as used herein refers to a substance which helps absorption of the elements of the pharmaceutical composition, stabilizes said elements, activates or helps preparation of the composition. Thus, examples of excipients used in parenteral formulations include, but are not limited to, antimicrobial agents (e.g., benzalkonium chloride, metacresol, thimerosal), co-solvents (e.g., ethanol), buffers, tonicity agents (e.g., NaCl) and pH adjusting factors (e.g., carbonate, citrate, phosphate solutions).

As is the case for the excipient, the “pharmaceutically acceptable vehicle” is a substance used in the composition to dilute any of the components contained therein to a determined volume or weight (e.g., a 0.9% (w/v) NaCl aqueous solution). The pharmaceutically acceptable vehicle is an inert substance or a substance with an analogous action to any of the elements comprising the pharmaceutical composition of the present invention. The role of said vehicle is to allow the incorporation of other elements, allow better dosing and administration or to provide consistency and shape to the composition.

Pharmaceutical compositions can comprise from approximately 1% to approximately 95% active ingredient. In some aspects, e.g., the pharmaceutical compositions of the present invention can comprise from approximately 20% to approximately 90% active ingredient (i.e., an IP5 substituted compound of the present invention or a combination thereof, alone or in combination, e.g., with one or more additional therapeutic agents).

Formulations of a pharmaceutical composition suitable for parenteral administration comprise the active ingredient, e.g., an IP5 substituted compound of the present invention (e.g., a compound selected from the group consisting of Compound 1 to Compound 53), combined with a pharmaceutically acceptable carrier, such as sterile water or sterile isotonic saline (e.g., a 0.9% (w/v) NaCl aqueous solution). Such formulations can be prepared, packaged, or sold in a form suitable for bolus administration or for non-bolus administration. Injectable formulations can be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations can further comprise one or more additional ingredients including, but not limited to, suspending, stabilizing, or dispersing agents.

In some aspects, in a formulation for parenteral administration, the active ingredient, e.g., an IP5 substituted compound of the present invention (e.g., a compound selected from the group consisting of Compound 1 to Compound 53), is provided in dry (i.e., powder or granular) form for reconstitution with a suitable vehicle (e.g., sterile pyrogen-free water) prior to parenteral administration of the reconstituted composition.

The pharmaceutical compositions can be prepared, packaged, or sold in the form of a sterile injectable aqueous or oily suspension or solution. This suspension or solution can be formulated according to the known art, and may comprise, in addition to the active ingredient (e.g., an inositol phosphate of the present invention), additional ingredients such as the dispersing agents, wetting agents, or suspending agents described herein. Such sterile injectable formulations can be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides.

Other administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt. Compositions and methods of making formulations for administering the IP5 substituted compounds of the present invention, including controlled- or sustained-release formulations containing the said active agents, are described in the art. See, e.g., Remington: The Science and Practice of Pharmacy, 23^{rd} Ed., 2021, Academic Press; U.S. Pat. Nos. 6,340,475, 6,488,962, 6,451,808, 5,972,389, 5,582,837, and 5,007,790; US20030147952, US20030104062, US20030104053, US20030044466, US20030039688, and US20020051820; WO2003035041, WO2003035040, WO2003035029, WO2003035177, WO2003035039, WO2002096404, WO2002032416, WO2001097783, WO2001056544, WO2001032217, WO1998055107, WO1998011879, WO1997047285, WO1993018755, and WO1990011757.

Medicaments according to the invention are manufactured by methods known in the art, especially by conventional mixing, coating, granulating, dissolving or lyophilizing.

The present invention also provides a compound or a combination of compounds or pharmaceutical formulation according to any of the above aspects of the invention, in the broadest definition given, or as specified in any of the aspects presented above, for use as a medicament.

The present invention also provides a compound or combination of compounds or pharmaceutical formulation according to any of the above aspects of the invention, in the broadest definition given, or as specified in any of the aspects presented above, for use in the treatment and/or prevention of a disease or condition disclosed herein.

The present invention also provides a compound or combination of compounds or pharmaceutical formulation according to any of the above aspects of the invention, in the broadest definition given, or as specified in any of the aspects presented above, for the manufacture of a medicament for the prevention and/or treatment of a disease or condition disclosed herein.

### IV. Articles of Manufacture and Kits

The present invention also provides articles of manufacture and kits. Such articles of manufacture and kits can comprise a container (e.g., a box) comprising one or more vials containing a formulation comprising one or more of the IP5 substituted compounds of the present invention (e.g., a compound selected from the group consisting of Compound 1 to Compound 53) and/or solvents for their medical administration or other uses according to the methods disclosed herein.

A kit or article of manufacture provided according to this invention can also comprise brochures or instructions describing the process of medical administration and dosages disclosed herein, or the use of the IP5 substituted compounds of the present invention (e.g., a compound selected from the group consisting of Compound 1 to Compound 53) according to the methods disclosed herein. In some aspects, kit or article of manufacture can comprise multiple vials, each one of them containing a single dose. In other aspects, kit or article of manufacture can comprise one or more vials, each one of them comprising more than one dose.

In some aspects, the article of manufacture is a bag containing a solution of an IP5 substituted compound of the present invention (e.g., a compound selected from the group consisting of Compound 1 to Compound 53). In other aspects, the article of manufacture is a bottle (e.g., a glass bottle or a plastic bottle) containing a solution of an IP5 substituted compound of the present invention (e.g., a compound selected from the group consisting of Compound 1 to Compound 53). In some aspects, the article of manufacture is a bag containing an IP5 substituted compound of the present invention (e.g., a compound selected from the group consisting of Compound 1 to Compound 53) in powder form for reconstitution in an appropriate solvent. In other aspects, the article of manufacture is a bottle (e.g., a glass bottle or a plastic bottle) containing an IP5 substituted compound of the present invention (e.g., a compound selected from the group consisting of Compound 1 to Compound 53) in powder form for reconstitution in an appropriate solvent.

The kits and articles of manufacture can include instructions for carrying out one or more administrations of the IP5 substituted compound of the present invention (e.g., a compound selected from the group consisting of Compound 1 to Compound 53) according to the methods and dosages disclosed herein.

Instructions included in the kits and articles of manufacture can be affixed to packaging material or can be included as a package insert. While the instructions are typically written or printed materials, they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### V. Uses of IP5 Substituted Compounds of Formula I

The present invention provides methods for using the IP5 substituted compounds of the present invention (e.g., a compound selected from the group consisting of Compound 1 to Compound 53) to treat diseases and conditions. In some aspects, the medical uses disclosed herein relate to the ability of the IP5 substituted compounds of the present invention (e.g., a compound selected from the group consisting of Compound 1 to Compound 53) to inhibit the formation or growth of calcium salts/crystals (e.g., calcium phosphates, HAP) in a subject in need thereof comprising administering to the subject an effective amount of a compound of formula I, or a pharmaceutical composition of disclosed herein. Accordingly, the IP5 substituted compounds of the present invention (e.g., a compound selected from the group consisting of Compound 1 to Compound 53) can be used to treat and prevent diseases and conditions related to pathological crystallization.

The present invention provides methods to treat and/or prevent pathological crystallizations and/or the consequences thereof in a subject in need thereof comprising administering an IP5 substituted compound of the present invention (e.g., a compound selected from the group consisting of Compound 1 to Compound 53), wherein the administration of the IP5 substituted compound effectively treats and/or prevents pathological crystallization and/or the consequences thereof in the subject.

The present invention also provides methods to manufacture a medicament for the treatment of pathological crystallization comprising using an intermediate compound selected from the group consisting of the compounds listed in Table 1. Also provided is a compound selected from the group consisting of Compound 1 to Compound 53 for the treatment of pathological crystallizations and/or the consequences thereof in a subject in need thereof.

In some aspects, the IP5 substituted compound of the present invention (e.g., a compound selected from the group consisting of Compound 1 to Compound 53) can be administered topically, enterally or parenterally.

### A. Synthesis of Protected Myo-Inositol Agents, Alkylating or Click Agents and Activated Acids

### A.1. Synthesis of Protected Myo-Inositol Agents (1), (2), (3), (4), (31), (32), (33), and (34)

1,3,5-O-Methylidyne-myo-inositol (1): The synthesis of (1) was previously described in the literature (Martin S, et al., J. Org. Chem. 1994; 59(17):4805-4820)

2-O-tert-Butyldimethylsilyl-1,3,5-O-methylidyne-myo-inositol (2): The synthesis of (2) was previously described in the literature (Kadirvel, 2008, supra).

4,6-bis-O-(4-Methoxybenzyl)-1,3,5-O-methylidyne-myo-inositol (3): The synthesis of (3) was previously described in the literature (Aiba, 2016, supra).

4,6-di-O-Benzyl-1,3,5-O-methylidyne-myo-inositol (4): The synthesis of (4) was previously described in the literature (Chen, 2015, supra).

2,4,6-tri-O-Benzyl-1,3,5-O-methylidyne-myo-inositol (31): The synthesis of (31) was previously described in the literature (Song F, et al., Org Biomol Chem 2012; 10:3642-3654).

2,4,6-tri-O-Benzyl-myo-inositol (32): The synthesis of (32) was previously described in the literature (Chen, 2015, supra).

1,2,3,4,6-penta-O-Benzyl-myo-inositol (33): The synthesis of (33) was previously described in the literature (Gurale B, et al., Carbohydrate Res 2018; 461:38-44).

rac-2,4,6-tri-O-Benzyl-3,5-O-ethylidene-myo-inositol (34): The synthesis of (34) was previously described in the literature (Song, 2012, supra).

### A.2. Alkylating or Click Agents' Synthesis

1-(4-(2-Chloroethyl)piperazin-1-yl)ethanone (5): To a solution of 1-(4-(2-hydroxyethyl)piperazin-1-yl)ethanone (1.74 g, 10.10 mmol) in dichloromethane (DCM, 0.2 M) at 0 °C, triethylamine (TEA, 2.1 mL, 15.15 mmol) and Ts-Cl (2.31 g, 12.12 mmol) were added. The reaction mixture was stirred for 72 h at room temperature and concentrated in vacuo. The residue was purified by flash chromatography (silica gel, DCM:MeOH (MeOH) (95:5) rending 1.28 g of (5) (66% yield). HPLC-MS (Condition A): rt=0.24 min; m/z: 191, 193 [M, M+2]⁺.

### 9-Methoxynonyl 4-methylbenzenesulfonate (7)

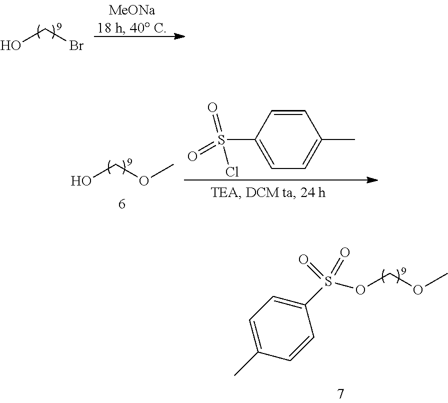

Step 1: 9-Methoxynonan-1-ol (6): A mixture of 9-bromononan-1-ol (0.5 g, 2.24 mmol) and sodium methoxide 4 N (25 mL, 100 mmol)) was stirred for 18 h at 40 °C. The reaction mixture was filtered and concentrated in vacuum. The residue was purified by flash chromatography (silica gel, hexane (Hex):ethyl acetate (EtOAc) 4:1) to afford 324 mg of (6) (83% yield). 1H NMR (400 MHz, Chloroform-d) δ 3.63 (t, J=6.6 Hz, 2H), 3.36 (t, J=6.6 Hz, 2H), 3.33 (s, 3H), 1.59-1.52 (m, 4H), 1.29 (m, 10H).

Step 2: 9-Methoxynonyl 4-methylbenzenesulfonate (7): To a solution of (6) (1.5 g, 8.61 mmol) in (28.7 mL), TEA (1.80 mL, 12.91 mmol) and Ts-Cl (2.13 g, 11.19 mmol) were added. The reaction mixture was stirred for 24 h at rt then quenched with water and washed with brine. The organic layer was dried over with Na₂SO₄, filtered and concentrated in vacuum. The residue was purified by flash chromatography (silica gel, Hex:EtOAc 4:1) to afford 2.15 g of (7) (76%). 1H NMR (400 MHz, Chloroform-d) δ 7.78 (d, J=8.3 Hz, 2H), 7.34 (d, J=8.3 Hz, 2H), 4.01 (t, J=6.5 Hz, 2H), 3.35 (t, J=6.6 Hz, 2H), 3.32 (s, 3H), 2.45 (s, 3H), 1.61 (dt, J=8, 6.6 Hz, 2H), 1.56-1.51 (m, 2H), 1.32-1.22 (m, 10H).

### 19-Methoxynonadec-10-yn-1-yl 4-methylbenzenesulfonate (13)

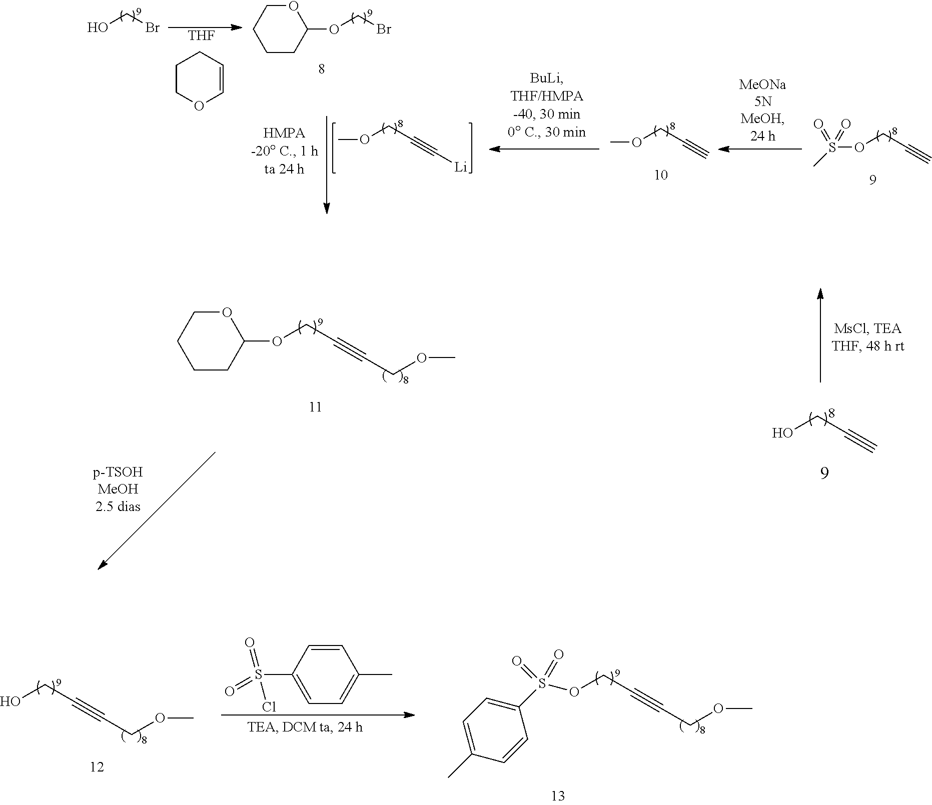

Step 1: 2-((9-Bromononyl)oxy)tetrahydro-2H-pyran (8): To a mixture of 9-bromononan-1-ol (2.18 g, 9.77 mmol) and p-TsOH (37 mg, 0.19 mmol), 3,4-dihydro-2H-pyran (1.3 mL, 14.65 mmol) was added. The reaction mixture was stirred for 3.5 days at 60 °C. The mixture was diluted with water, extracted with ethyl ether (2×), dried over with Na₂SO₄, filtered, and the solvents removed in vacuo. The residue was purified by flash chromatography (silica gel, Hex:EtOAc 9:1) rending 2.4 g of (8) (80% yield). ¹H-NMR was identical to the previously described in the literature (Grube A, et al., Eur J Org Chem 2006; 1285-1295).

Step 2: Dec-9-yn-1-yl methanesulfonate (9): To a solution of dec-9-yn-1-ol (5.22 g, 33.8 mmol) in THF (0.35M) at 0 °C, TEA (6.3 mL, 44 mmol) and Ms-Cl (3.5 mL, 44 mmol) were added. The reaction mixture was stirred for 16 h at room temperature and was diluted with water, extracted with DCM (×2), dried over with Na₂SO₄, filtered, and the solvents removed in vacuo. The residue was purified by flash chromatography (silica gel, Hex:EtOAc (20:1) rending 8.1 g of (9) (>99% yield). ¹H-NMR was identical to the previously described in the literature (Langmuir: the ACS journal of surfaces and colloids., 2013, Vol. 29(2), p. 570-580).

Step 3: 10-Methoxydec-1-yne (10): To a (9) (8.17 g, 35.2 mmol), a solution 5 M of sodium methoxide in MeOH (35.2 mL, 176 mmol) was added. The reaction mixture was stirred for 18 h days at 45 °C. The mixture was diluted with water, extracted with ethyl ether (2×), dried over with Na₂SO₄, filtered, and the solvents removed in vacuo. The residue was purified by flash chromatography (silica gel, Hex:EtOAc 20:1) rending 4 g of (10) (67% yield). ¹H NMR (400 MHz, Chloroform-d) δ 3.34 (td, J=6.6, 1.0 Hz, 2H), 3.31 (s, 3H), 2.15 (tdd, J=7.1, 2.7, 1.0 Hz, 2H), 1.91 (td, J=2.7, 1 Hz, 1H), 1.62-1.45 (m, 4H), 1.41-1.23 (m, 8H).

Step 4: 2-((19-Methoxynonadec-10-yn-1-yl)oxy)tetrahydro-2H-pyran (11): In an tetrahydrofurane (THF) and hexamethylphosphoramide (HMPA) mixed solution (1.2:1) of (10) (1.41 g, 8.42 mmol) cooled to −40 °C, an Hex. solution 1.6M of n-BuLi (7.45 mL, 11.93 mmol) was slowly added, followed by stirring at the same temperature for 30 minutes and further stirring at 0 °C for 30 minutes. After cooling to −20 °C, a HMPA solution of (8) (2.16 g, 7.02 mmol) was slowly added. After stirring at the same temperature for 10 minutes, the temperature was increased to room temperature. Stirring was conducted at the same temperature for 22 hours. Under ice cooling (at 4 °C), an aqueous 1N HCl solution and tBuMeO were added, followed by washing in turn with NaHCO₃ sat solution and saturated brine and further drying over anhydrous Na₂SO₄. After filtration, the solvent was distilled off under reduced pressure. The residue was purified by flash chromatography (silica gel, Hex:EtOAc 10:1) rending 1.28 g of (11) (46% yield). ¹H NMR (400 MHz, Chloroform-d) δ 4.55 (dd, J=4.4, 2.8 Hz, 1H), 3.85 (ddd, J=10.8, 7.6, 3.6 Hz, 1H), 3.71 (dt, J=9.6, 6.8 Hz, 1H), 3.51-3.45 (m, 1H), 3.35 (dt, J=9.6, 6.8 Hz, 1H), 3.34 (t, J=6.8 Hz, 2H), 3.31 (s, 3H), 2.14-2.08 (m, 4H), 1.82 (tdd, J=10.8, 7.6, 4.6 Hz, 1H), 1.71 (dt, J=12.8, 2.8 Hz, 1H), 1.63-1.40 (m, 10H), 1.39-1.21 (m, 16H), 0.90-0.79 (m, 4H).

Step 5: 19-Methoxynonadec-10-yn-1-ol (12): To a solution of (11) (3.28 g, 8.31 mmol) in MeOH (0.6M), p-TsOH (95 mg, 0.5 mmol) was added. The reaction mixture was stirred for 3.5 days at 60 °C. The mixture was diluted with water, extracted with ethyl ether (2×), dried over with Na₂SO₄, filtered, and the solvents removed in vacuo. The filtrate was concentrated yielding 2.6 g of (12) (>99% yield). ¹H NMR (400 MHz, Chloroform-d) 3.61 (t, J=6.6 Hz, 2H), 3.34 (t, J=6.6 Hz, 2H), 3.31 (s, 3H), 2.11 (t, J=7.2 Hz, 4H), 1.56-1.30 (m, 26H).

Step 6: 19-Methoxynonadec-10-yn-1-yl 4-methylbenzenesulfonate (13): To a solution of (12) (2.6 g, 8.37 mmol) in DCM (0.5M) at 0 °C, TEA (1.4 mL, 10.05 mmol), DMAP (102 mg, 0.83 mmol) and Ts-Cl (1.92 g, 10.05 mmol) were added. The reaction mixture was stirred for 6 h at room temperature and concentrated in vacuo. The residue was purified by flash chromatography (silica gel, Hex:EtOAc 10:1) rending 2.45 g of (13) (63% yield). HPLC-MS (Condition B): rt=5.20 min; m/z: 465 [M+1]⁺, 482 [M+23]⁺.

### 29-methoxynonacosa-9,20-diyn-1-yl 4-methylbenzenesulfonate (16)

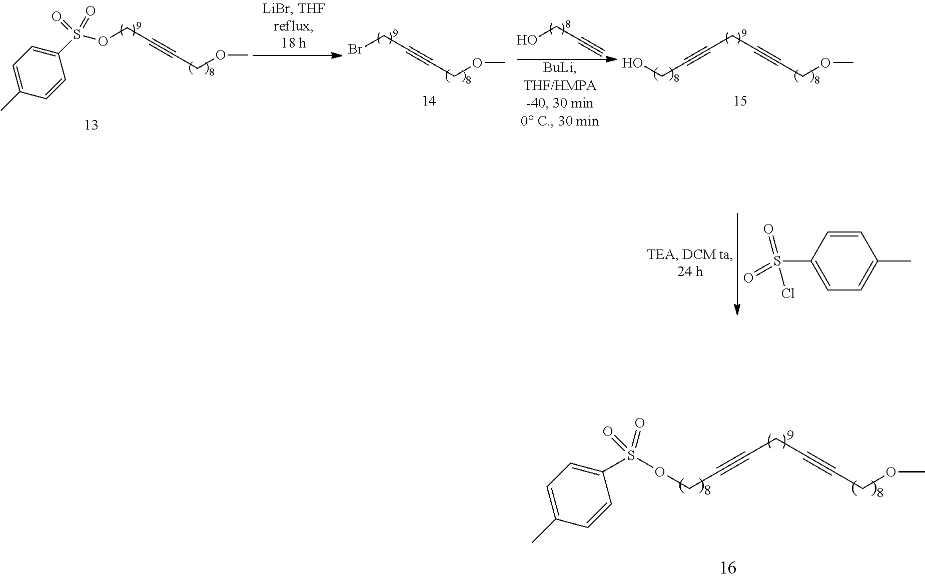

Step 1:19-Bromo-1-methoxynonadec-9-yne (14): A solution of lithium bromide (0.318 g, 3.66 mmol) in anhydrous THF (0.1M) was dried with 3A molecular sieves (300 mg, previously activated at 400 °C for 24 hours) placed inside the reaction flask. After stirring this solution for 20 minutes, a solution of (13) (0.85 g, 1.829 mmol) in anhydrous THF (2 mL+2 mL) was added to the previous reaction flask. This solution was stirred at reflux for 4 h. Then, the reaction mixture was allowed to reach room temperature and it was diluted with tBuMeO. The organic layer was washed with sat aq NaHCO₃, dried over Na₂SO₄, filtered and the solvent was removed under reduced pressure to afford a yellow oil. The residue was purified by flash chromatography (silica gel, Hex:EtOAc 10:1) rending 467 mg of (14) (68% yield). ¹H NMR (400 MHz, Chloroform-d) δ 3.39 (t, J=6.8 Hz, 2H), 3.34 (t, J=6.8 Hz, 2H), 3.31 (s, 3H), 2.12 (t, J=6.6 Hz, 4H), 1.83 (q, J=7.2 Hz, 2H), 1.57-1.51 (m, 2H), 1.49-1.22 (m, 24H).

Step 2: 29-Methoxynonacosa-9,20-diyn-1-ol (15): In an THF/HMPA mixed solution (1.2:1) of dec-9-yn-1-ol (230 mg, 1.48 mmol) cooled to −40 °C, an n-hexane solution 1.6M of n-BuLi (1.9 mL, 3.09 mmol) was slowly added, followed by stirring at the same temperature for 30 minutes and further stirring at 0 °C for 30 minutes. After cooling to −20 °C, a HMPA solution of (14) (462 mg, 1.24 mmol) was slowly added. After stirring at the same temperature for 10 minutes, the temperature was increased to room temperature. Stirring was conducted at the same temperature for 22 hours. Under ice cooling (at 4 °C), an aqueous 1N HCl solution and tBuMeO were added, followed by washing in turn with NaHCO₃ sat solution and saturated brine and further drying over anhydrous Na₂SO₄. After filtration, the solvent was distilled off under reduced pressure. The residue was purified by flash chromatography (silica gel+5% of AgNO₃, Hex:EtOAc 4:1) rending 69.7 mg of (15) (12% yield). ¹H NMR (400 MHz, Deuterium Oxide) δ 3.64 (t, J=6.6 Hz, 2H), 3.36 (t, J=6.6 Hz, 2H), 3.33 (s, 3H), 2.13 (t, J=7.0 Hz, 8H), 1.58-1.52 (d, J=8.2 Hz, 4H), 1.51-1.41 (m, 8H), 1.41-1.23 (m, 26H).

Step 3: 29-Methoxynonacosa-9,20-diyn-1-yl 4-methylbenzenesulfonate (16): To a solution of (15) (69.7 mg, 0.156 mmol) in DCM (0.1M) at 0 °C, TEA (43 uL, 0.325 mmol), DMAP (3.3 mg, 0.027 mmol) and Ts-Cl (62 mg, 0.325 mmol) were added. Then, the reaction mixture was diluted with water and tBuMeO. The organic layer was washed with sat aq NaHCO₃ and brine, dried over Na₂SO₄, filtered and the solvent was removed under reduced pressure to afford a yellow oil. The residue was purified by flash chromatography (silica gel, Hex:EtOAc 10:1) rending 63.3 mg of (16) (67% yield). ¹H NMR (400 MHz, Chloroform-d) δ 7.77 (d, J=8.0 Hz, 2H), 7.32 (d, J=8.0 Hz, 2H), 3.99 (t, J=6.6 Hz, 2H), 3.34 (t, J=6.6 Hz, 2H), 3.30 (s, 3H), 2.43 (s, 3H), 2.12-2.10 (m, 8H), 1.66-1.49 (m, 4H), 1.48-1.39 (m, 8H), 1.39-1.12 (m, 26H).

Bromo-7-methoxyheptane (17): A mixture of 1,7-dibromoheptane (3.27 mL, 19.38 mmol) and sodium methoxide 5N (3.88 mL, 19.38 mmol) was stirred for 4 h at 40 °C. The reaction mixture was filtered and concentrated in vacuum. The residue was purified by flash chromatography (silica gel, Hex:EtOAc 10:1) to afford 1.4 g of (17) (34.5%). ¹H NMR (400 MHz, Chloroform-d) δ 3.40 (t, J=6.9 Hz, 2H), 3.36 (t, J=6.6 Hz, 2H), 3.33 (s, 3H), 1.89-1.82 (m, 2H), 1.60-1.53 (m, 2H), 1.47-1.40 (m, 2H), 1.39-1.32 (m, 4H).

3-Azidopropyl 4-methylbenzenesulfonate (18): To a solution of 3-azidopropan-1-ol (1 g, 9.89 mmol) in DCM (0.2M) at 0 °C, TEA (2.07 mL, 14.84 mmol) and Ts-Cl (2.26 g, 11.87 mmol) were added. The reaction mixture was stirred for 24 h at rt then quenched with water and washed with brine. The organic layer was dried over with Na₂SO₄, filtered and concentrated in vacuum. The residue was purified by flash chromatography (silica gel, Hex:EtOAc 4:1) to afford 1.27 g of (18) (50%). ¹H NMR (400 MHz, Chloroform-d) δ 7.80 (d, J=8.3 Hz, 2H), 7.36 (dd, J=8.3, 0.7 Hz, 2H), 4.11 (t, J=6.3 Hz, 2H), 3.38 (t, J=6.3 Hz, 2H), 2.46 (s, 3H), 1.89 (p, J=6.3 Hz, 2H).

1-(5-Bromopentyl)-1H-pyrazole (19): To a mixture of 1H-pyrazole (0.78 g, 11.49 mmol) and Cs₂CO₃ (3.74 mg, 11.49 mmol) in acetonitrile (CAN, 57 mL) 1,5-dibromopentane (1.56 mL, 11.49 mmol) was added. The mixture was stirred for 18 h at rt. The reaction mixture was filtered and concentrated in vacuum. The residue was purified by flash chromatography (silica gel, Hex:EtOAc 1:1) to afford 1.1 g of (19) (45%). HPLC-MS (Condition A): rt=3.00 min; m/z: 218, 220 [M+1, M+3]⁺.

Benzyl pent-4-ynoate (20): A solution of pent-4-ynoic acid (5.02 g, 51.2 mmol) in DMF (102 mL) was treated with K₂CO₃ (10.61 g, 77 mmol)) and benzyl bromide (6.09 mL, 51.2 mmol) stirred for 72 h, then diluted with water (200 mL) and extracted with diethyl ether (3×150 mL). The combined organic extracts were dried (MgSO₄) and filtered. The solvents were removed in vacuo to afford 9.9 g of (20) (>99%). HPLC-MS (Condition A): rt=3.70 min.

### 6-Azidohexyl 4-methylbenzenesulfonate (22)

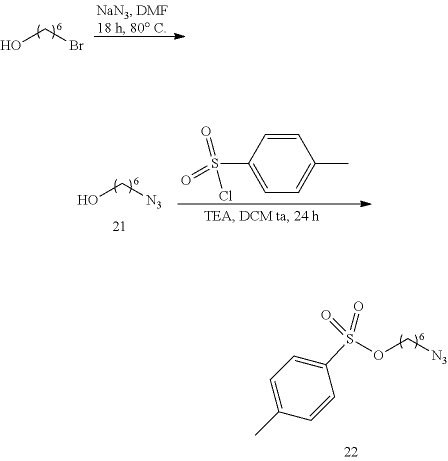

Step 1: 6-Azidohexan-1-ol (21): A solution of 6-bromohexan-1-ol (428 μL, 3.27 mmol) and sodium azide (850 mg, 13.08 mmol) in DMF (0.8 M) was stirred for 18 h at 80 °C. Then, the reaction mixture was quenched with water/EtOAc, and washed with brine (3×). The organic layer was dried over with Na₂SO₄, filtered, and concentrated in vacuum to afford 470 mg of (21) (>99%). ¹H NMR (400 MHz, Chloroform-d) δ 3.65 (t, J=6.8 Hz, 2H), 3.27 (t, J=6.8 Hz, 2H), 1.70-1.56 (m, 4H), 1.40 (m, 4H).

Step 2: 6-Azidohexyl 4-methylbenzenesulfonate (22): At 0 °C, p-Ts-Cl (720 mg, 3.78 mmol) was added to a solution of (21) (515 mg, 3.60 mmol) and TEA (1.5 mL, 10.79 mmol) in dry DCM (0.6 M). The reaction mixture was stirred overnight at rt. It was diluted with EtOAc and washed with 10% aqueous solution of NaHSO₄. The aqueous phase was extracted with EtOAc (3×). The combined layers were washed with saturated aqueous solution of NaHCO₃ and dried over anhydrous Na₂SO₄. The solvent was removed in vacuo and the residue was purified by flash chromatography (silica gel Hex:EtOAc 6:1) giving 666 mg of (22) (62%). ¹H NMR (400 MHz, Chloroform-d) δ 7.79 (d, J=8.0 Hz, 2H), 7.35 (d, J=8.0 Hz, 2H), 4.03 (t, J=6.4 Hz, 2H), 3.23 (t, J=6.8 Hz, 2H), 2.45 (s, 3H), 1.66 (p, J=6.4 Hz, 2H), 1.55 (p, J=6.4 Hz, 2H), 1.41-1.18 (m, 4H).

2-Cyclopropylethyl 4-methylbenzenesulfonate (23): To a solution of 2-cyclopropylethanol (3 mL, 34.0 mmol) in DCM (85 mL), TEA (7.10 mL, 50.9 mmol) and Ts-Cl (7.77 g, 40.8 mmol) were added, and the obtained mixture was stirred for 18 h. Water was added to the reaction mixture and extracted with DCM (2×). The combined organic layer was washed with brine, dried over Na₂SO₄, and concentrated in vacuo. The residue was purified by column chromatography on silica gel (Hex-EtOAc, 4:1) yielding 6.23 g (76%) of (23). ¹H NMR (400 MHz, Chloroform-d) δ 7.78 (d, J=8.2 Hz, 2H), 7.32 (d, J=8.2 Hz, 2H), 4.06 (t, J=6.8 Hz, 2H), 2.43 (s, 3H), 1.51 (q, J=6.8 Hz, 2H), 0.63 (m, 1H), 0.38 (ddd, J=8, 6, 4 Hz, 2H), −0.01 (dt, J=6, 4 Hz, 2H).

2-Cyclopentylethyl 4-methylbenzenesulfonate (24): To a solution of 2-cyclopentylethanol (1.09 mL, 8.76 mmol) in DCM (22 mL), TEA (1.83 mL, 13.1 mmol) and Ts-Cl (2.00 g, 10.5 mmol) were added, and the obtained mixture was stirred for 18 h. Water was added to the reaction mixture and extracted with DCM (2×). The combined organic layer was washed with brine, dried over with Na₂SO₄, and concentrated in vacuo. The residue was purified by column chromatography on silica gel (Hex-EtOAc, 4:1) yielding 1.53 g (65%) of (24). ¹H NMR (400 MHz, Chloroform-d) δ 7.81 (d, J=8 Hz, 2H), 7.37 (d, J=8 Hz, 2H), 4.06 (t, J=6.7 Hz, 2H), 2.47 (s, 3H), 1.89-1.76 (m, 1H), 1.76-1.43 (m, 8H), 1.10-0.99 (m, 2H).

3-(4-Methoxyphenyl)propyl 4-methylbenzenesulfonate (25): To a solution of 3-(4-methoxyphenyl)propan-1-ol (0.96 mL, 6.02 mmol) in DCM (15 mL), TEA (1.26 mL, 9.02 mmol) and Ts-Cl (1.38 g, 7.22 mmol) were added, and the obtained mixture was stirred for 18 h. Water was added to the reaction mixture and extracted with DCM (2×). The combined organic layer was washed with brine, dried over with Na₂SO₄, and concentrated in vacuo. The residue was purified by column chromatography on silica gel (Hex-EtOAc, 4:1) yielding 1.59 g (82%) of (25). ¹H NMR (400 MHz, Chloroform-d) δ 7.81 (d, J=8 Hz, 2H), 7.37 (d, J=8 Hz, 2H), 7.00 (d, J=8.6 Hz, 2H), 6.80 (d, J=8.6 Hz, 2H), 4.04 (t, J=6 Hz, 2H), 3.80 (s, 3H), 2.61 (dd, J=8.2, 6.8 Hz, 2H), 2.48 (s, 3H), 2.03-1.86 (ddt, J=8.2, 6.8, 6 Hz, 2H).

3-(3-(Trifluoromethyl)phenyl)propyl 4-methylbenzenesulfonate (26): To a solution of 3-(3-(trifluoromethyl)phenyl)propan-1-ol (0.91 mL, 4.90 mmol) in DCM (12 mL), TEA (1.02 mL, 7.35 mmol) and Ts-Cl (1.12 g, 5.88 mmol) were added, and the obtained mixture was stirred for 18 h. Water was added to the reaction mixture and extracted with DCM (2×). The combined organic layer was washed with brine, dried over with Na₂SO₄, and concentrated in vacuo. The residue was purified by column chromatography on silica gel (Hex-EtOAc, 4:1) yielding 1.53 g (87%) of (26). ¹H NMR (400 MHz, Chloroform-d) δ 7.63 (d, J=8.5 Hz, 2H), 7.28 (d, J=7.8 Hz, 1H), 7.21-7.16 (m. 2H), 7.19 (d, J=8.5 Hz, 2H), 7.12 (J=7.8 Hz, 1H), 3.88 (t, J=6.2 Hz, 2H), 2.56 (dd, 8.0, 7.6 Hz, 2H), 2.29 (s, 3H), 1.81 (ddt, J=8.0, 7.6, 6.2 Hz, 2H).

3-(p-Tolyl)propyl 4-methylbenzenesulfonate (27): To a solution of 3-(p-tolyl)propan-1-ol (1 g, 6.66 mmol) in DCM (17 mL), TEA (1.39 mL, 9.99 mmol) and Ts-Cl (1.52 g, 7.99 mmol) were added, and the obtained mixture was stirred for 18 h. Water was added to the reaction mixture and extracted with DCM (2×). The combined organic layer was washed with brine, dried over with Na₂SO₄, and concentrated in vacuo. The residue was purified by column chromatography on silica gel (Hex-EtOAc, 4:1) yielding 1.85 g (87%) of (27). ¹H NMR (400 MHz, Chloroform-d) δ 7.81 (d, J=8.0 Hz, 2H), 7.37 (d, J=8.0 Hz, 2H), 7.07 (d, J=8.0 Hz, 2H), 6.98 (d, J=8.0 Hz, 2H), 4.05 (t, J=6.4 Hz, 2H), 2.62 (t, J=7.5 Hz, 2H), 2.48 (s, 3H), 2.32 (s, 3H), 1.95 (tt, J=7.5, 6.4 Hz, 2H).

### 5-acetamidopentyl 4-methylbenzenesulfonate (29)

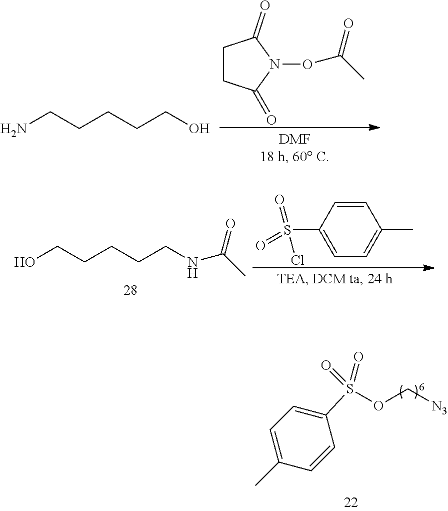

Step 1: N-(5-hydroxypentyl)acetamide (28): To a solution of 5-aminopentan-1-ol (50 g, 485 mmol) and 2,5-dioxopyrrolidin-1-yl acetate (75 g, 485 mmol) in dimethylformamide (DMF, 0.5 M), TEA (101 mL, 727 mmol) was added and the solution was stirred for 18 h at 60 °C. Then, the solvent was evaporated via a high-vacuum rotary evaporator, and residue was dissolved MeOH and treated with IRA-410 till basic pH (approx 10), filtered, and evaporated to dryness to give 70.4 g of (28) (100%). This crude was used without further purification. ¹H NMR (400 MHz, CD₃OD) δ 3.57 (t, J=6.5 Hz, 2H), 3.18 (t, J=7.0 Hz, 2H), 1.94 (s, 3H), 1.65-1.47 (m, 4H), 1.49-1.32 (m, 2H).

Step 2: 5-Acetamidopentyl 4-methylbenzenesulfonate (29): To a solution of (28) (70.4 g, 485 mmol) in DCM (0.5 M), TEA (101 mL, 727 mmol) and Ts-Cl (92 g, 485 mmol) were added. The reaction mixture was stirred for 18 h at rt, then, quenched with water/DCM and washed with NaHCO₃ sat and brine. The organic layer was dried over with Na₂SO₄, filtered and concentrated in vacuum to afford 92.45 g of (29) (63%). ¹H NMR (400 MHz, Chloroform-d): δ 7.84-7.74 (m, 2H), 7.37 (d, J=8.1 Hz, 2H), 5.59 (s, 1H), 4.04 (t, J=6.3 Hz, 2H), 3.21 (q, J=6.8 Hz, 2H), 2.47 (s, 3H), 1.98 (s, 3H), 1.68 (dt, J=7.8, 6.6 Hz, 2H), 1.57-1.43 (m, 2H), 1.39 (qd, J=7.3, 3.0 Hz, 2H). HPLC-MS (Condition A): rt=3.42 min; m/z: 300 [M+1]⁺.

(((10-Bromodecyl)oxy)methyl)benzene (30): The synthesis of (4) was previously described in the literature (Hanbali M., et al., Bioorg Med Chem Letters 2006; 16(10):2637-2640).

5-(benzyloxy)pentyl 4-methylbenzenesulfonate (35): To a solution of 5-(benzyloxy)pentan-1-ol (11.2 g, 57.7 mmol) in dichloromethane (DCM, 0.4 M) at 0 °C, triethylamine (TEA, 12 mL, 86 mmol) and Ts-Cl (11 g, 57.7 mmol) were added. The reaction mixture was stirred for 18 h at room temperature and concentrated in vacuo. The residue was purified by flash chromatography (silica gel, Hex/OEtAc (4:1)) rending 15.32 g of (35) (76% yield). HPLC-MS (Condition A): rt=4.55 min; m/z: 349 [M+1]⁺, 371 [M+23]⁺

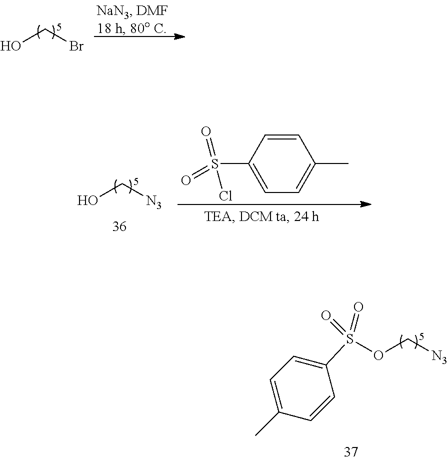

### 5-Azidopentyl 4-methylbenzenesulfonate (37)

Step 1: 5-Azidopentan-1-ol (36): A solution of 5-bromopentan-1-ol (0.10 mL, 0.89 mmol) and sodium azide (63 mg, 0.98 mmol) in dimethylformamide (DMF, 0.2 M) was stirred for 18 h at 80 °C. Then, the reaction mixture was quenched with water/EtOAc and was washed with brine (3×). The organic layer was dried over with Na₂SO₄, filtered, and concentrated in vacuum to afford 57 mg of (36) (50%). ¹H NMR (400 MHz, Chloroform-d) δ 3.63 (t, J=6.8 Hz, 2H), 3.26 (t, J=6.8 Hz, 2H), 1.61 (p, J=7.2 Hz, 2H), 1.58 (p, J=6.8 Hz, 2H), 1.52-1.36 (m, 2H).

Step 2: 5-Azidopentyl 4-methylbenzenesulfonate (37) *csc_san_02_N37: To a solution of (36) (54 mg, 0.42 mmol) in DCM (0.2 M) at 0 °C, TEA (87 μL, 0.63 mmol) and Ts-Cl (80 mg, 0.42 mmol) were added. The reaction mixture was stirred for 60 h at rt, and then quenched with water/DCM and washed with brine. The organic layer was dried over with Na₂SO₄, filtered and concentrated in vacuum to afford 92 mg of (37) (78%). HPLC-MS (Condition A): rt=3.00 min; m/z: 284 [M+1]⁺.

Methyl 3-azidopropanoate (38): A mixture of methyl 3-bromopropanoate (10.70 ml, 98 mmol) and sodium azide (8.92 g, 137 mmol) were dissolved in DMSO (Vol: 49.0 ml). The solution was heated to 45 °C and stirred for 18 h. After the mixture was cooled down to room temperature, and extracted with diethyl ether. The combined organic layer was dried over anhydrous magnesium sulfate, filtered, and concentrated on a rotary evaporator to give azidopropanoate as colorless liquid. ¹H NMR (400 MHz, Chloroform-d) δ 3.62 (s, 3H), 3.47 (t, J=6.3 Hz, 2H), 2.87 (t, J=6.6 Hz, 2H).

### B. General Procedures

### B.1. Alkylation Procedures

Procedure A: To a solution of (2), (3), (4), (33) or (34) (1 eq) in dimethylformamide (DMF, 0.2 M) at 0 °C, sodium hydride (2.15 eq) was added. When the addition was complete, the mixture was stirred for 5 minutes at room temperature. Finally, the alkylating agent was added. The reaction was allowed to stir overnight, then quenched with water and extracted with EtOAc. The organic layer was dried over Na₂SO₄, filtered and concentrated in vacuum. The residue was purified by flash chromatography (silica gel) to afford pure compounds.

Procedure B: To a solution of (2), (3), (4), (33) or (34) (1 eq) in DMF (c=0.15 M) at 0 °C, sodium hydride (1.2 eq) was added. When the addition was complete, the mixture was stirred for 5 min at room temperature. Finally, alkylation agent was added. The reaction was allowed to stir overnight, then quenched with water and extracted with EtOAc. The organic layer was dried over Na₂SO₄, filtered and concentrated in vacuum. The residue was purified by flash chromatography (silica gel) to afford pure compounds.

Procedure N: To a solution of (2), (3), (4), (33) or (34) (1 eq) in DMF (0.2 M) at 0 °C, LiH was added. When the addition was complete, the mixture was stirred for 5 minutes at rt. Finally, the alkylating agent was added. The reaction was allowed to stir during different times and temperatures depending on the alkylating agent, then quenched with water, and extracted with EtOAc. The organic layer was dried over with Na₂SO₄, filtered, and concentrated in vacuum. The residue was purified by flash chromatography (silica gel) to afford pure compounds.

### B.2. Debenzylation Procedures

Procedure K: Alkyl-inositol derivative (I_B′, II_C, VIII_B′ or IX_D) was dissolved in a mixture of THF/MeOH (7:3, 0.05M) followed by addition of excess palladium hydroxide on carbon. The mixture was placed under hydrogen atmosphere and stirred 2 days at room temperature. The mixture was then purged with nitrogen, filtered and concentrated to afford the desired compounds.

### B.3. Hydrolysis Procedures

Procedure C: Alky-Inositol derivative (II_A, II_B, II_B′, II_D, V_A, V_B, V_D or VIII_B) was dissolved in a mixture of MeOH/Water/DCM/trifluoroacetic acid (3:1:1:1, 0.1M) and the solution was stirred at room temperature overnight. Finally, the solvent and excess of TFA was removed under vacuum to afford the desiderated compounds.

Procedure D: Alky-Inositol derivative (II_A, II_B, II_B′, II_D, V_A, V_B, V_D or VIII_B) was dissolved in a mixture of MeOH/Water/DCM/trifluoroacetic acid (3:1:1:1, 0.1M) and the solution was stirred at room temperature overnight. Then, the solvent and excess of TFA was removed under vacuum. Finally, the residue was dissolved in MeOH, treated with IRA-400 resin until pH was basic, filtered and the solvent was removed under vacuum to afford the desiderated compounds.

Procedure E: To a solution of alkyl-inositol derivative (II_A or V_A) in tetrahydrofuran (THF) (c=0.1M), a solution 0.1M of TBAF in THF (1.2 eq) was added. The reaction mixture was stirred for 18 h at room temperature, quenched with water and extracted with EtOAc. The organic layer was dried over Na₂SO₄, filtered and concentrated in vacuum. The residue was dissolved in a mixture of MeOH/Water/DCM/trifluoroacetic acid (3:1:1:1, 0.1M) and the solution was stirred at room temperature overnight. Then, the solvent and excess of TFA was removed under vacuum. Finally, the residue was dissolved in MeOH (MeOH), treated with IRA-400 resin until pH was basic, filtered and the solvent was removed under vacuum to afford the desiderated compounds.

Procedure F: alkyl-inositol derivative (II_A, II_B, II_B′, II_D, V_A, V_B, V_D or VIII_B) was dissolved in a mixture of DCM/trifluoroacetic acid (4:1, 0.14 M) and the solution was stirred at room temperature 1 h. Then, water was added (final concentration 0.1M) and the reaction mixture was stirred at room temperature 18 h. Finally, layers were separated, and aqueous layer was concentrated in vacuum to afford the desiderated compound.

Procedure S: alkyl-inositol derivative (II_A, II_B, II_B′, II_D, V_A, V_B, V_D or VIII_B) was dissolved in a mixture of MeOH/HCl 1N (10:1, 0.1 M) and the solution was stirred at rt or 60 °C 48 h. Finally, the solvent and excess of HCl were removed under vacuum. Finally, the residue was dissolved in MeOH, treated with IRA-400 resin until pH was basic, filtered and the solvent was removed under vacuum to afford the desiderated compounds.

### B.4. Click Reaction Procedures

Procedure M: To a solution of Intermediate (VI_A, V_C or X_D) (1 eq), CuSO₄·5H₂O (0.4 eq) and sodium ascorbate (0.6 eq) in a mixture water (0.07 M), a solution of alkynyl reagent in THF (0.35 M) was added. The reaction mixture was stirred at rt for 24 h at 60 °C under inert atmosphere, filtered over celite, and concentrated in vacuum. The residue was dissolved in MeOH, treated with IRA-400 resin until pH was basic, filtered, and the solvent was removed under vacuum to afford the desired compounds.

Procedure L: To a solution of Intermediate (III_B or IX_D) (1 eq), CuSO₄·5H₂O (0.4 eq) and sodium ascorbate (0.6 eq) in water (0.07M), a solution of azide-reagent (2 eq) in THF (0.35 M) was added. The reaction mixture was stirred at rt for 20 h under inert atmosphere, filtered over celite, and concentrated in vacuo. The residue was treated with water/DCM. Then, the organic layer was dried over with Na₂SO₄, filtered, and concentrated in vacuum to afford the desired compound.

### B.5. Azide Reduction Procedure

Procedure P: To a solution of Intermediate (V_B or X_D) (1 eq) in a mixture of THF/water (9:1, 0.08M), DIPEA (3 eq) and PPh₃ (2.6 eq) was added. The reaction mixture was stirred for 4 h at 60 °C, then quenched with water, and washed with brine. The organic layer was dried over with Na₂SO₄, filtered, and concentrated in vacuum to afford the desired compound. No purification was performed.

### B.6. Amide Formation Procedure

Procedure Q: To a solution of Intermediate (II_A, III_B or IX_D) (1 eq) in DMF (0.1 M), TEA (4 eq) and activated acid (2 eq) were added. The reaction mixture was stirred for 4 h at rt and concentrated in vacuum. The residue was dissolved in MeOH, treated with IRA-400 resin until pH was basic, filtered, and the solvent was removed under vacuum. Finally, the residue was purified by flash chromatography (silica gel) to afford pure compounds.

### B.5. Phosphorylation Procedures

Procedure G: alkyl-inositol derivative (III_A, VI_A, III_B, III_C or III_D) (1 eq) was dissolved in DCM (0.02M) and a solution on tetrazole in ACN (0.43M) (14.4 eq) was added. The mixture was stirred 30 min. at room temperature. Then N,N-diethyl-1,5-dihydrobenzo[e][1,3,2]-dioxaphosphepin-3-amine (7.2 eq) was added and the was stirred at room temperature overnight. Finally, the reaction mixture was cooled at 0 °C and a solution of tert-butyl hydroperoxide in hexane (5.5M) (19.2 eq) was added. The solution was brought to room temperature and stirred for 1 h. The mixture was washed with dilute sodium sulfite and extracted with DCM. The organic layer was dried over Na₂SO₄, filtered and concentrated in vacuum. The residue was purified by flash chromatography (silica gel) to afford pure compounds.

Procedure H: Alky-Inositol derivative (III_A, VI_A, III_B, III_C or III_D) (1 eq) was dissolved in DCM (0.02M) and a solution on tetrazole in ACN (0.43M) (18 eq) was added. The mixture was stirred 30 min at room temperature. Then, N,N-diethyl-1,5-dihydrobenzo[e][1,3,2]-dioxaphosphepin-3-amine (9 eq) was added and the was stirred at room temperature overnight. Finally, the reaction mixture was cooled at 0 °C and a solution of tert-butyl hydroperoxide in hexane (5.5M) (24 eq) was added. The solution was brought to room temperature and stirred for 1 h. The mixture was washed with dilute sodium sulfite and extracted with DCM. The organic layer was dried over Na₂SO₄, filtered and concentrated in vacuum. The residue was purified by flash chromatography (silica gel) to afford pure compounds.

Procedure R: Alky-Inositol derivative (III_A, VI_A, III_B, III_C or III_D) (1 eq) was dissolved in DMF (0.05M) and a solution on phenyltetrazole in DMF (5M) (18 eq) was added. The mixture was stirred 30 min at room temperature. Then, N,N-diethyl-1,5-dihydrobenzo[e][1,3,2]-dioxaphosphepin-3-amine (9 eq) was added and the was stirred at room temperature overnight. Finally, the reaction mixture was cooled at 0 °C and a solution of tert-butyl hydroperoxide in hexane (5.5M) (24 eq) was added. The solution was brought to room temperature and stirred for 1 h. The mixture was washed with dilute sodium sulfite and extracted with EtOAc (×2). The organic layer was dried over Na₂SO₄, filtered and concentrated in vacuum. The residue was purified by flash chromatography (silica gel) to afford pure compounds.

### B.6. Phosphate Deprotection Procedures

Procedure I: Phosphorylated compound (IV_A, VII-A, IV_B, IV_C or IV_D) was dissolved in a mixture of THF/MeOH/Water (3:1:1, 0.01M) followed by addition of excess palladium hydroxide on carbon or palladium on carbon. The mixture was placed under hydrogen atmosphere and stirred 2 days at room temperature. The mixture was then purged with nitrogen, filtered and concentrated. The compound was brought at pH 7 by addition of dilute aqueous NaOH (1N) and the residue was purified on a sephadex column (PD-10, G-25-M) by eluting with water. All fractions were lyophilized and analyzed by ¹H-RMN. The fractions containing product were purified further on a reverse phase cartridge (Sep-Pack, Waters, 1 g, C18) by eluting with water. All fractions were lyophilized and analyzed by ¹H-RMN.

Procedure J: Phosphorylated compound (IV_A, VII-A, IV_B, IV_C or IV_D) was dissolved in a mixture of THF/MeOH/Water (3:1:1, 0.01M) followed by addition of excess palladium hydroxide on carbon or palladium on carbon. The mixture was placed under hydrogen atmosphere and stirred 2 days at room temperature. The mixture was then purged with nitrogen, filtered and concentrated. The compound was brought at pH 10 by addition of dilute aqueous NaOH (1N) and the solution was stirred for 24-48 h. Finally, the solution was purified on a sephadex column (PD-10, G-25-M) by eluting with water. All fractions were lyophilized and analyzed by ¹H-RMN. The fractions containing product were purified further on a reverse phase cartridge (Sep-Pack, Waters, 1 g, C18) by eluting with water. All fractions were lyophilized and analyzed by ¹H-RMN.

Procedure T: Phosphorylated compound (IV_A, VII-A, IV_B, IV_C or IV_D) was treated with thiophenol (40 eq), m-cresol (40 eq) in TFA (0.045 M). Then TBMSBr (40 eq) was added slowly and the mixture was stirred at rt for 4 h, quenched with water, and extracted with DCM (3×). The aqueous layer was concentrated in vacuum. The residue was brought at pH 9-10 by addition of water and dilute aqueous NaOH (1N), and the compound was purified on a sephadex column (PD-10, G-25-M) by eluting with water. All fractions were lyophilized and analyzed by ¹H-RMN. The fractions containing product were purified further in a reverse phase cartridge (Sep-Pack® C18 cartridge, 1 g, Waters Corp., Milford, MA, USA) by eluting with water. All fractions were lyophilized and analyzed by ¹H-RMN.

### C. Synthesis of Intermediates II-X

### C.1. Intermediates II_A, II_B, II_B′, II_C and II_D

rac-2-O-tert-Butyldimethylsilyl-1,3,5-O-methylidyne-4-O-pentyl-myo-inositol (II_A-1): According to general alkylation Procedure A, from 377 μL (3 eq) of 1-bromopentane, 48 mg of (II_A-1) were obtained (13% yield). HPLC-MS (Condition A): rt=4.90 min; m/z: 375 [M+1]⁺.

rac-4-O-(5-(Benzyloxy)pentyl)-2-O-tert-butyldimethylsilyl-1,3,5-O-methylidyne-myo-inositol (II_A-2): According to general alkylation Procedure A, from 1.04 mL (3 eq) of (((5-bromopentyl)oxy)methyl)benzene, 183 mg of (II_A-2) were obtained (22.8% yield). HPLC-MS (Condition A): rt=4.99 min; m/z: 481 [M+1]⁺, 503 [M+23]⁺.

rac-2-O-tert-Butyldimethylsilyl-4-O-(5-methoxypentyl)-1,3,5-O-methylidyne-myo-inositol (II_A-3): According to general alkylation Procedure A, from 241 mg (2.2 eq, in 3 mL of DMF) of 1-bromo-5-methoxypentane, 26 mg of (II_A-3) were obtained (11% yield). HPLC-MS (Condition B): rt=3.22 min; m/z: 405 [M+1]⁺.

rac-2-O-tert-Butyldimethylsilyl-4-O-(5-ethoxycarbonylpentyl)-1,3,5-O-methylidyne-myo-inositol (II_A-4): According to general alkylation Procedure A, from 351 μL of ethyl 6-bromohexanoate (3 eq), 107 mg of (II_A-4) were obtained (36% yield). HPLC-MS (Condition A): rt=4.99 min; m/z: 447 [M+1]⁺.

rac-4-O-(2-(4-Acetylpiperazin-1-yl)ethyl)-2-O-tert-butyldimethylsilyl-1,3,5-O-methylidyne-myo-inositol (II_A-5): According to general alkylation Procedure A, from 700 mg (2.5 eq in 7.3 mL of DMF) of (5) (2.5 eq, in 7.3 mL of DMF), 460 mg of (II_A-5) were obtained (68% yield). HPLC-MS (Condition A): rt=1.90 min; m/z: 459 [M+1]⁺.

4,6-bis-O-(4-Methoxybenzyl)-1,3,5-O-methylidyne-2-O-pentyl-myo-inositol (II_B-1): According to general alkylation Procedure B, from 200 mg of (3) and 87 μL (1.5 eq) of 1-bromopentane, 142 mg (II_B-1) were obtained (61% yield). HPLC-MS (Condition B): rt=3.43 min; m/z: 501 [M+1]⁺, 523[M+23]⁺.

4,6-bis-O-(4-Methoxybenzyl)-2-O-(5-methoxypentyl)-1,3,5-O-methylidyne-myo-inositol (II_B-2): According to general alkylation Procedure B, from 400 mg of (3) and 505 mg (3 eq) of 1-bromo-5-methoxypentane, 180 mg of (II_B-2) were obtained (36.5% yield). HPLC-MS (Condition A): rt=4.26 min; m/z: 531 [M+1]⁺, 553 [M+23]⁺.

4,6-bis-O-(4-Methoxybenzyl)-2-O-(9-methoxynonyl)-1,3,5-O-methylidyne-myo-inositol (II_B-3): According to general alkylation Procedure B, from 500 mg of (3) and 420 mg (1.1 eq) of (7), 430 mg of (II_B-3) were obtained (63% yield). ¹H NMR (400 MHz, Chloroform-d) δ 7.20 (d, J=8.6 Hz, 4H), 6.82 (d, J=8.6 Hz, 4H), 5.48 (d, J=1.2 Hz, 1H), 4.60 (d, J=11.3 Hz, 2H), 4.47 (d, J=11.3 Hz, 2H), 4.38-4.35 (m, 1H), 4.30 (t, J=3.6 Hz, 2H), 4.24-4.22 (m, 2H), 3.82 (q, J=1.6 Hz, 1H), 3.79 (s, 6H), 3.47 (t, J=6.8 Hz, 2H), 3.34 (t, J=6.8 Hz, 2H), 3.31 (s, 3H), 1.64-1.50 (m, 8H), 1.33-1.21 (m, 6H).

4,6-di-O-Benzyl-2-O-(19-methoxynonadec-10-yn-1-yl)-1,3,5-O-methylidyne-myo-inositol (IIB′-1): According to general alkylation Procedure B, from 437 mg of (4) and 631 mg (1.1 eq) of (13), 504 mg of (II_B′-1) were obtained (66% yield). ¹H NMR (400 MHz, Chloroform-d) δ 7.28 (s, 10H), 5.49 (d, J=1.3 Hz, 1H), 4.68 (d, J=11.8 Hz, 3H), 4.55 (d, J=11.8 Hz, 2H), 4.45-4.40 (m, 1H), 4.34 (t, J=3.6 Hz, 2H), 4.27 (m, 2H), 3.85 (q, J=1.6 Hz, 1H), 3.48 (t, J=6.8 Hz, 2H), 3.34 (t, J=6.8 Hz, 2H), 3.31 (s, 3H), 2.11 (t, J=7.0 Hz, 4H), 1.60 (q, J=7.0 Hz, 2H), 1.54-1.51 (m, 2H), 1.44 (q, J=7.0 Hz, 4H), 1.37-1.24 (m, 16H).

4,6-di-O-Benzyl-2-O-(29-methoxynonacosa-9,20-diyn-1-yl)-1,3,5-O-methylidyne-myo-inositol (II_B′-2): According to general alkylation Procedure B, from 404 mg of (4) and 721 mg (1.1 eq) of (16), 776 mg of (II_B′-2) were obtained (89% yield). ¹H NMR (400 MHz, Chloroform-d) δ 7.28 (s, 10H), 5.49 (d, J=1.3 Hz, 1H), 4.68 (d, J=11.8 Hz, 2H), 4.55 (d, J=11.8 Hz, 2H), 4.44-4.42 (m, 1H), 4.34 (t, J=3.7 Hz, 2H), 4.27 (m, 2H), 3.85 (q, J=1.7 Hz, 1H), 3.47 (t, J=6.8 Hz, 2H), 3.34 (t, J=6.8 Hz, 2H), 3.31 (s, 3H), 2.11 (t, J=7.2 Hz, 8H), 1.61 (p, J=6.9 Hz, 2H), 1.55-1.50 (m, 2H), 1.45 (p, J=7.0 Hz, 8H), 1.38-1.25 (m, 26H).

rac-2-O-tert-Butyldimethylsilyl-1,3,5-O-methylidyne-4-O-(3-(p-tolyl)propyl)-myo-inositol (II_A-6): According to general alkylation Procedure N, from 1.848 g (2.5 eq.) of (27), 374 mg of (II_A-6) were obtained (35% yield). HPLC-MS (Condition A): rt=4.99 min; m/z: 437 [M+1]⁺, 459 [M+23]⁺

rac-2-O-tert-Butyldimethylsilyl-1,3,5-O-methylidyne-4-O-(4-methylpentyl)-myo-inositol (II_A-7): According to general alkylation Procedure N, from 1 mL (2.5 eq.) of 1-bromo-4-methylpentane, 781 mg of (II_A-7) were obtained (71% yield). HPLC-MS (Condition A): rt=5.09 min; m/z: 389 [M+1]⁺, 411 [M+23]⁺.

rac-2-O-tert-Butyldimethylsilyl-1,3,5-O-methylidyne-4-O-(6,6,6-trifluorohexyl)-myo-inositol (II_A-8): According to general alkylation Procedure N, from 1.158 g (2.5 eq.) of 6-bromo-1,1,1-trifluorohexane, 574 mg of (II_A-8) were obtained (61% yield). HPLC-MS (Condition A): rt=4.73 min; m/z: 443 [M+1]⁺, 465 [M+23]⁺.

rac-2-O-tert-Butyldimethylsilyl-4-O-(3-(4-methoxyphenyl)propyl)-1,3,5-O-methylidyne-myo-inositol (II_A-9): According to general alkylation Procedure N, from 1.59 g (2.5 eq.) of (25), 457 mg of (II_A-9) were obtained (51% yield). HPLC-MS (Condition A): rt=4.72 min; m/z: 453 [M+1]⁺, 455 [M+23]⁺.

rac-2-O-tert-Butyldimethylsilyl-1,3,5-O-methylidyne-4-O-(3-(3-(trifluoromethyl)phenyl)propyl)-myo-inositol (II_A-10): According to general alkylation Procedure N, from 1.53 g (2.5 eq.) of (26), 807 mg of (II_A-10) were obtained (96% yield). HPLC-MS (Condition A): rt=4.99 min; m/z: 491 [M+1]⁺.

rac-2-O-tert-Butyldimethylsilyl-4-O-(2-cyclopentylethyl)-1,3,5-O-methylidyne-myo-inositol (II_A-11): According to general alkylation Procedure N, from 1.53 g (2.5 eq.) of (24), 50 mg of (11_A-11) were obtained (5% yield). HPLC-MS (Condition A): rt=5.26 min; m/z: 401 [M+1]⁺, 423 [M+23]⁺.

rac-2-O-tert-Butyldimethylsilyl-4-O-(2-cyclopropylethyl)-1,3,5-O-methylidyne-myo-inositol (II_A-12): According to general alkylation Procedure A, from 1.45 g (1 eq.) of (23), 552 mg of (II_A-12) were obtained (25% yield). HPLC-MS (Condition A): rt=4.65 min; m/z: 373 [M+1]⁺, 395 [M+23]⁺.

rac-2-O-tert-Butyldimethylsilyl-1,3,5-O-methylidyne-4-O-(5-(1H-pyrazol-1-yl)pentyl)-myo-inositol (II_A-13): According to general alkylation Procedure A, from 214 mg of (19), 174 mg of (11_A-13) were obtained (60% yield). HPLC-MS (Condition A): rt=4.11 min; m/z: 441 [M+1]⁺.

rac-4-O-(5-Acetamidopentyl)-2-O-tert-butyldimethylsilyl-1,3,5-O-methylidyne-myo-inositol (II_A-14): According to general alkylation Procedure N, from 1 g (3 eq.) of (29), 241 mg of (II_A-14) were obtained (50% yield). HPLC-MS (Condition A): rt=3.78 min; m/z: 432 [M+1]⁺.

rac-2-O-tert-Butyldimethylsilyl-4-O-(10-ethoxycarbonyldecyl)-1,3,5-O-methylidyne-myo-inositol (II_A-15): According to general alkylation Procedure A, from 500 mg (2.5 eq.) of ethyl 11-bromoundecanoate, 50 mg of (II_A-15) were obtained (14% yield). ¹H NMR (400 MHz, Chloroform-d) δ 1H NMR (400 MHz, Chloroform-d) δ 5.47 (d, J=1.6 Hz, 1H), 4.39 (ddt, J=10.1, 4.1, 2.8 Hz, 1H), 4.24 (t, J=2.8 Hz, 2H), 4.18 (dt, J=5.2, 2.1 Hz, 1H), 4.14 (q, J=1.6 Hz, 1H), 4.11-4.08 (m, 1H), 4.09 (q, J=7.1 Hz, 1H), 3.70 (d, J=10.1 Hz, 1H), 3.59 (td, J=9.5, 6.5 Hz, 1H), 3.58 (td, J=9.5, 6.5 Hz, 1H), 2.25 (t, J=7.6 Hz, 2H), 1.58 (p, J=6.8 Hz, 2H), 1.55 (p, J=6.8 Hz, 2H), 1.30-1.23 (m, 12H), 1.22 (t, J=7.1 Hz, 3H), 0.92 (s, 9H), 0.13 (s, 6H).

rac-4-O-(10-(Benzyloxy)decyl)-2-O-tert-butyldimethylsilyl-1,3,5-O-methylidyne-myo-inositol (II_A-16): According to general alkylation Procedure A, from 407.9 mg (2.1 eq.) of (30), 44.5 mg of (II_A-16) were obtained (14% yield). HPLC-MS (Condition A): rt=6.13 min; m/z: 551 [M+1]⁺.

rac-2-O-tert-Butyldimethylsilyl-4-O-methyl-1,3,5-O-methylidyne-myo-inositol (II_A-17): According to general alkylation Procedure A, from 0.1 mL (1 eq.) of methyl iodide, 50 mg of (11_A-17) were obtained (10% yield). HPLC-MS (Condition A): rt=3.83 min; m/z: 319 [M+1]⁺, 341 [M+23]⁺.

rac-2-O-tert-Butyldimethylsilyl-4-O-(7-methoxyheptyl)-1,3,5-O-methylidyne-myo-inositol (II_A-18): According to general alkylation Procedure A, from 780 mg (2.5 eq.) of (17), 163 mg of (II_A-18) were obtained (25% yield). HPLC-MS (Condition A): rt=4.79 min; m/z: 433 [M+1]⁺, 455 [M+23]⁺.

rac-2-O-tert-Butyldimethylsilyl-1,3,5-O-methylidyne-4-O-propyl-myo-inositol (II_A-19): According to general alkylation Procedure A, from 481 uL (5 eq.) of 1-iodopropane, 139 mg of (II_A-19) were obtained (41% yield). HPLC-MS (Condition A): rt=4.41 min; m/z: 347 [M+1]⁺, 369 [M+23]⁺.

1,2,3,4,6-penta-O-Benzyl-5-O-propargyl-myo-inositol (II_C-1): According to general alkylation Procedure B, from 200 mg of (33) and 141 uL 3-bromoprop-1-yne in toluene (3 eq, 80%), 107 mg of (II_C-1) were obtained (50% yield). HPLC-MS (Condition A): rt=5.98 min; m/z: 670 [M+1]⁺, 692 [M+23]⁺.

1,2,3,4,6-penta-O-Benzyl-5-O-(2-cyclopropylethyl)-myo-inositol (II_C-2): According to general alkylation Procedure B, from 200 mg of (33) and 114 mg (1.5 eq) of (23), 108 mg of (II_C-2) were obtained (49% yield). HPLC-MS (Condition A): rt=6.56 min; m/z: 700 [M+1]⁺, 722 [M+23]⁺.

1,2,3,4,6-penta-O-Benzyl-5-O-(9-methoxynonyl)-myo-inositol (II_C-3): According to general alkylation Procedure B, from 200 mg of (33) and 156 mg (1.5 eq) of (7), 127 mg of (II_C-3) were obtained (51% yield). HPLC-MS (Condition A): rt=7.11 min; m/z: 789[M+1]⁺, 810 [M+23]⁺.

rac-2,4,6-tri-O-Benzyl-3,5-O-ethylidene-1-O-propargyl-myo-inositol (II_D-1): According to general alkylation Procedure B, from 162 mg of (34) and 152 uL 3-bromoprop-1-yne in toluene (3 eq, 80%), 146 mg of (II_C-1) were obtained (83% yield). HPLC-MS (Condition A): rt=5.08 min; m/z: 537 [M+23]⁺.

rac-2-O-tert-Butyldimethylsilyl-4-O-(5-carboxypentyl)-1,3,5-O-methylidyne-myo-inositol (II_A-20): To a solution of Intermediate (II_A-4) (1.58 g, 3.56 mmol) in MeOH/THF (0.15 M), a solution of lithium hydroxide (1M, 11 mL, 10.96 mmol) was added and the solution was stirred for 18 h at rt, then, quenched with HCl 1M/DCM and washed with brine. The organic layer was dried over with Na₂SO₄, filtered and concentrated in vacuum. The residue was purified by flash chromatography (silica gel, Hex/EtOAc 1:1) to afford 575 mg of (II_A-20) (38%). HPLC-MS (Condition A): rt=3.91 min; m/z: 419 [M+1]⁺.

rac-4-O-(6-Amino-6-oxohexyl)-2-O-tert-butyldimethylsilyl-1,3,5-O-methylidyne-myo-inositol (II_A-21): To a mixture of Intermediate (II_A-20) (80 mg, 0.19 mmol), TEA (53 uL, 0.38 mmol) and bis(2,5-dioxopyrrolidin-1-yl) carbonate (59 mg, 0.23 mmol) in DCM (0.15 M), a solution of ammonia in dioxane (2.8 mL, 1.91 mmol) was added and the solution was stirred for 18 h at rt, then, quenched with water/DCM and washed with brine. The organic layer was dried over with Na₂SO₄, filtered and concentrated in vacuum to afford 72 mg of (II_A-21) (90%). HPLC-MS (Condition A): rt=3.66 min; m/z: 418 [M+1]⁺.

4,6-bis-O-(4-Methoxybenzyl)-2-O-propargyl-myo-inositol (II_B-4): According to general alkylation Procedure A, from 248 mg of (3) and 171 mg (2 eq) of propargyl bromide, 184 mg of (II_B-4) were obtained (68% yield). HPLC-MS (Condition A): rt=4.07 min; m/z: 469 [M+1]⁺.

2-O-(5-(Benzyloxy)pentyl)-4,6-bis-O-(4-methoxybenzyl)-1,3,5-O-methylidyne-myo-inositol (II_B-5): According to general alkylation Procedure A, from 162 mg of (3) and 262 mg (2 eq) of (35), 179 mg of (II_B-5) were obtained (78% yield). HPLC-MS (Condition A): rt=4.90 min; m/z: 607 [M+1]⁺, 629 [M+23]⁺.

2-O-(5-Aminopentyl)-4,6-bis-O-(4-methoxybenzyl)-1,3,5-O-methylidyne-myo-inositol (II_B-6): According to general azide reduction Procedure P, from 110 mg of (V_B-1), 203 mg of (II_B-6) were obtained (100%). HPLC-MS (Condition A): rt=3.24 min; m/z: 516 [M+1]⁺.

4,6-bis-O-(4-Methoxybenzyl)-2-O-(5-(4-(methoxymethyl)-1H-1,2,3-triazol-1-yl)pentyl)-1,3,5-O-methylidyne-myo-inositol (II_B-7): According to general click reaction Procedure M, from 126 mg of (V_B-1) and 16 mg (1 eq.) of 3-methoxyprop-1-yne, 142 mg of (II_B-7) were obtained (100%). HPLC-MS (Condition A): rt=3.97 min; m/z: 612 [M+1]⁺.

4,6-di-O-Benzyl-2-O-(6,6,6-trifluorohexyl)-O-1,3,5-methylidyne-myo-inositol (II_B′-3): According to general alkylation Procedure B, from 200 mg of (4) and 142 mg (1.2 eq) of 6-bromo-1,1,1-trifluorohexane, 89.9 mg of (II_B′-3) were obtained (33% yield). HPLC-MS (Condition A): rt=4.90 min; m/z: 509 [M+1]⁺, 532 [M+23]⁺.

2-O-(6-Amino-6-oxohexyl)-4,6-di-O-benzyl-1,3,5-O-methylidyne-myo-inositol (IIB′-4): According to general alkylation Procedure B, from 200 mg of (4) and 126 mg (1.2 eq) of 6-bromohexanamide, 70 mg of (II_B′-4) were obtained (27% yield). HPLC-MS (Condition A): rt=3.75 min; m/z: 484 [M+1]⁺.

4,6-di-O-Benzyl-2-O-(2-cyclopentylethyl)-1,3,5-O-methylidyne-myo-inositol (II_B′-5): According to general alkylation Procedure B, from 175 mg of (4) and 152 mg (1.2 eq) of (24), 184 mg of (II_B′-5) were obtained (83% yield). HPLC-MS (Condition A): rt=5.20 min; m/z: 467 [M+1]⁺.

4,6-di-O-Benzyl-2-O-(2-cyclopropylethyl)-1,3,5-O-methylidyne-myo-inositol (II_B′-6): According to general alkylation Procedure B, from 200 mg of (4) and 195 mg (1.5 eq) of (23), 230 mg of (II_B′-6) were obtained (>99% yield). HPLC-MS (Condition A): rt=4.74 min; m/z: 439 [M+1]⁺, 461 [M+23]⁺.

1,2,3,4,6-penta-O-Benzyl-5-O-(5-methoxypentyl)-myo-inositol (II_C-4): According to general alkylation Procedure A, from 250 mg of (33) and 144 mg (1.5 eq) of 1-bromo-5-methoxypentane, 46 mg of (II_C-4) were obtained (16% yield). HPLC-MS (Condition A): rt=6.36 min; m/z: 732 [M+1]⁺, 754 [M+23]⁺.

1,2,3,4,6-penta-O-Benzyl-5-O-(6,6,6-trifluorohexyl)-myo-inositol (II_C-5): According to general alkylation Procedure A, from 250 mg of (33) and 174 mg (2 eq) of 6-bromo-1,1,1-trifluorohexane, 162 mg of (II_C-5) were obtained (53% yield). HPLC-MS (Condition A): rt=6.38 min; m/z: 770 [M+1]⁺, 792 [M+23]⁺.

1,2,3,4,6-penta-O-Benzyl-5-O-(5-(4-(methoxymethyl)-1,2,3-triazol-1-yl)pentyl)-myo-inositol (II_C-6): According to general click reaction Procedure M, from 110 mg of (V_C-1) and 21 mg (2 eq.) of 3-methoxyprop-1-yne, 102 mg of (II_C-6) were obtained (85%). HPLC-MS (Condition A): rt=5.59 min; m/z: 813 [M+1]⁺.

rac-2,4,6-tri-O-Benzyl-3,5-O-ethylidene-1-O-(9-methoxynonyl)-myo-inositol (II_D-2): According to general alkylation Procedure B, from 203 mg of (34) and 210 mg (1.5 eq) of (7), 270 mg of (II_D-2) were obtained (>99% yield). HPLC-MS (Condition A): rt=6.06 min; m/z: 656 [M+23]⁺.

rac-2,4,6-tri-O-Benzyl-1-O-(2-cyclopropylethyl)-3,5-O-ethylidene-myo-inositol (II_D-3): According to general alkylation Procedure B, from 215 mg of (34) and 163 mg (1.5 eq) of (23), 246 mg of (II_D-3) were obtained (>99% yield). HPLC-MS (Condition A): rt=5.53 min; m/z: 568 [M+23]⁺.

rac-2,4,6-tri-O-Benzyl-3,5-O-ethylidene-2-O-propargyl-myo-inositol (II_D-4): According to general alkylation Procedure A, from 215 mg of (34) and 201 mg (3 eq) of propargyl bromide, 232 mg of (II_D-4) were obtained (>99% yield). HPLC-MS (Condition A): rt=4.97 min; m/z: 537 [M+23]⁺.

rac-2,4,6-tri-O-Benzyl-3,5-O-ethylidene-1-O-(6,6,6-trifluorohexyl)-myo-inositol (II_D-5): According to general alkylation Procedure A, from 250 mg of (34) and 230 mg (2 eq) of 6-bromo-1,1,1-trifluorohexane, 107 mg of (II_D-5) were obtained (33% yield). HPLC-MS (Condition A): rt=5.62 min; m/z: 637 [M+23]⁺.

rac-2,4,6-tri-O-Benzyl-1-O-(2-cyclopentylethyl)-3,5-O-ethylidene-myo-inositol (II_D-6): According to general alkylation Procedure A, from 250 mg of (34) and 211 mg (1.5 eq) of (24), 124 mg of (II_D-6) were obtained (41% yield). HPLC-MS (Condition A): rt=6.06 min; m/z: 596 [M+23]⁺.

### C.2. Intermediates III_A, III_B, III_C and III_D

rac-4-O-Pentyl-myo-inositol (III_A-1): According to general hydrolysis Procedure E, from 48 mg of (II_A-1), 16.6 mg of (III_A-1) were obtained (53% yield).

rac-4-O-(5-(Benzyloxy)pentyl)-myo-inositol (III_A-2): According to general hydrolysis Procedure C, from 48 mg of (II_A-2), 20.71 mg of (III_A-2) were obtained (58% yield). HPLC-MS (Condition B): rt=1.83 min; m/z: 357 [M+1]⁺.

rac-4-O-(5-Methoxypentyl)-myo-inositol (III_A-3): According to general hydrolysis Procedure C, from 26 mg (II_A-3), 12.3 mg of (III_A-3) were obtained (68% yield). HPLC-MS (Condition B): rt=0.53 min; m/z: 281 [M+1]⁺, 303 [M+23]⁺.

rac-4-O-(5-Methoxycarbonylpentyl)-myo-inositol (III_A-4): According to general hydrolysis Procedure C introducing a slight modification (aqueous work-up), from 100 mg of (II_A-4), 42 mg of (III_A-4) were obtained (61% yield). HPLC-MS (Condition B): rt=0.92 min; m/z: 309 [M+1]⁺.

rac-4-O-(2-(4-Acetylpiperazin-1-yl)ethyl)-myo-inositol (III_A-5): According to general hydrolysis Procedure C, from 38 mg of (II_A-5), 24 mg of (III_A-5) were obtained (86% yield). HPLC-MS (Condition B): rt=0.23 min; m/z: 335 [M+1]⁺.

2-O-Pentyl-myo-inositol (III_B-1): According to general hydrolysis Procedure F, from 127 mg of (II_B-1), 55.5 mg of (III_B-1) were obtained (87% yield). HPLC-MS (Condition A): rt=1.65 min; m/z: 251 [M+1]⁺, 273 [M+23]⁺.

2-O-(5-Methoxypentyl)-myo-inositol (III_B-2): According to general hydrolysis Procedure F, from 180 mg of (II_B-2), 84 mg of (III_B-2) were obtained (88% yield). ¹H NMR (400 MHz, MeOH-d₄) δ 374 (t, J=6.4 Hz, 2H), 3.67 (t, J=2.6 Hz, 1H), 3.56 (t, J=9.6 Hz, 2H), 3.37 (t, J=6.4 Hz, 2H), 3.35 (dd, 9.6, 2.6 Hz, 2H, 2H), 3.29 (s, 3H), 3.11 (t, J=9.6 Hz, 1H), 1.62-1.53 (m, 4H), 1.44-1.36 (m, 2H).

2-O-(9-Methoxynonyl)-myo-inositol (III_B-3): According to general hydrolysis Procedure F, from 430 mg of (II_B-3), 240 mg of (III_B-3) were obtained (97% yield). HPLC-MS (Condition B): rt=2.53 min; m/z: 337 [M+1]⁺.

2-O-(19-Methoxynonadecyl)-myo-inositol (III_B-4): According to general hydrolysis Procedure C, from 337 mg of (VIII_B-1), 200 mg of (III_B-4) were obtained (60% yield). HPLC-MS (Condition B): rt=4.43 min; m/z: 477 [M+1]⁺.

2-O-(29-Methoxynonacosyl)-myo-inositol (III_B-5): According to general hydrolysis Procedure C, from 284 mg of (VIII_B-2), 75 mg of (III_B-5) were obtained (27% yield).

rac-4-O-(3-(p-Tolyl)propyl)-myo-inositol (III_A-6): According to general hydrolysis Procedure S, from 344 mg of (II_A-6), 166.4 mg of (III_A-6) were obtained (67% yield). HPLC-MS (Condition A): rt=2.87 min; m/z: 313 [M+1]⁺.

rac-4-O-(4-Methylpentyl)-myo-inositol (III_A-7): According to general hydrolysis Procedure S, from 767 mg of (II_A-7), 225 mg of (III_A-7) were obtained (43% yield). HPLC-MS (Condition A): rt=2.60 min; m/z: 265 [M+1]⁺.

rac-4-O-(6,6,6-Trifluorohexyl)-myo-inositol (III_A-8): According to general hydrolysis Procedure S, from 424 mg of (II_A-8), 274 mg of (III_A-8) were obtained (90% yield). HPLC-MS (Condition A): rt=2.66 min; m/z: 319 [M+1]⁺.

rac-4-O-(3-(4-Methoxyphenyl)propyl)-myo-inositol (III_A-9): According to general hydrolysis Procedure S, from 409 mg of (II_A-9), 196 mg of (III_A-9) were obtained (66% yield). HPLC-MS (Condition A): rt=2.66 min; m/z: 329 [M+1]⁺.

rac-4-O-(3-(3-(Trifluoromethyl)phenyl)propyl)-myo-inositol (III_A-10): According to general hydrolysis Procedure S, from 209 mg of (II_A-10), 146 mg of (III_A-10) were obtained (93% yield). HPLC-MS (Condition A): rt=3.01 min; m/z: 367 [M+1]⁺.

rac-4-O-(2-Cyclopentylethyl)-myo-inositol (III_A-11): According to general hydrolysis Procedure S, from 68 mg of (11_A-11), 45 mg of (III_A-11) were obtained (96% yield). HPLC-MS (Condition A): rt=2.65 min; m/z: 277 [M+1]⁺.

rac-4-O-(2-Cyclopropylethyl)-myo-inositol (III_A-12): According to general hydrolysis Procedure S, from 486 mg of (II_A-12), 355 mg of (III_A-12) were obtained (96% yield). HPLC-MS (Condition A): rt=1.09 min; m/z: 249 [M+1]⁺.

rac-4-O-(5-(1H-Pyrazol-1-yl)pentyl)-myo-inositol (III_A-13): According to general hydrolysis Procedure S, from 137 mg of (II_A-13), 77 mg of (III_A-13) were obtained (78% yield). HPLC-MS (Condition A): rt=1.65 min; m/z: 317 [M+1]⁺.

rac-4-O-(5-Acetamidopentyl)-myo-inositol (III_A-14): According to general hydrolysis Procedure S, from 254 mg of (II_A-14), 180 mg of (III_A-14) were obtained (>99% yield). HPLC-MS (Condition A): rt=2.71 min; m/z: 308 [M+1]⁺.

rac-4-O-(10-Methoxycarbonyldecyl)-myo-inositol (III_A-15): According to general hydrolysis Procedure S, from 63 mg of (II_A-15), 30 mg of (III_A-15) were obtained (64% yield). HPLC-MS (Condition A): rt=3.11 min; m/z: 379 [M+1]⁺.

rac-4-O-(10-(Benzyloxy)decyl)-myo-inositol (III_A-16): According to general hydrolysis Procedure S, from 25 mg of (II_A-16), 19 mg of (III_A-16) were obtained (98% yield). HPLC-MS (Condition A): rt=3.54 min; m/z: 427 [M+1]⁺.

rac-4-O-Methyl-myo-inositol (III_A-17): According to general hydrolysis Procedure S, from 58 mg of (II_A-17), 27 mg of (III_A-17) were obtained (76% yield). ¹H NMR (400 MHz, Chloroform-d) δ 1H NMR (400 MHz, MeOH-d4) δ 3.87 (t, J=2.8 Hz, 1H), 3.55 (s, 3H), 3.36 (dd, ii=9.6, 2.8 Hz, 1H), 3.30-3.22 (m, 3H), 3.15 (t, J=9.2 Hz, 1H).

rac-4-O-(7-Methoxyheptyl)-myo-inositol (III_A-18): According to general hydrolysis Procedure S, from 167 mg of (II_A-18), 103 mg of (III_A-18) were obtained (87% yield). HPLC-MS (Condition A): rt=2.57 min; m/z: 309 [M+1]⁺.

rac-4-O-Propyl-myo-inositol (III_A-19): According to general hydrolysis Procedure S, from 128 mg of (II_A-19), 66 mg of (III_A-19) were obtained (80% yield). 1H NMR (400 MHz, MeOH-d4) δ 3.88 (t, J=2.4 Hz, 1H), 3.69 (td, J=9.2, 7.2 Hz, 1H), 3.68 (td, J=9.2, 7.2 Hz, 1H), 3.56 (t, J=9.5 Hz, 1H), 3.39-3.33 (m, 2H), 3.30-3.23 (m, 1H), 3.19-3.12 (m, 1H), 1.58 (h, J=7.2 Hz, 2H), 0.87 (t, J=7.2 Hz, 3H).

rac-4-O-(3-(4-(2-(Benzyloxycarbonyl)ethyl)-1H-1,2,3-triazol-1-yl)propyl)-myo-inositol (III_A-20): According to general click reaction Procedure M, from 57 mg of (VI_A-1) and 61.3 mg (1.5 eq.) of (20), 34 mg of (III_A-20) were obtained (35%). In this case, flash chromatography was employed. HPLC-MS (Condition A): rt=2.84 min; m/z: 452 [M+1]⁺.

rac-4-O-(6-(4-(Methoxycarbonyl)-1H-1,2,3-triazol-1-yl)hexyl)-myo-inositol (III_A-21): According to general click reaction Procedure M, from 99 mg of (VI_A-2) and 27 mg (1 eq.) of methyl propiolate, 109 mg of (III_A-21) were obtained (86%). HPLC-MS (Condition A): rt=2.39 min; m/z: 390 [M+1]⁺.

5-O-Propyl-myo-inositol (III_C-1): According to general debenzylation Procedure K, from 107 mg of (U C-1), 15 mg of (III_C-1) were obtained (42% yield). ¹H NMR (400 MHz MeOH-d4) δ 3.84 (t, J=2.8, 1H), 3.64 (t, J=7.4 Hz, 2H), 3.56 (t, J=9.6 Hz, 2H), 3.24 (dd, J=9.6, 2.8 Hz, 2H), 2.86 (t, J=9.6 Hz, 1H), 1.54 (q, J=7.4 Hz, 3H), 0.83 (t, J=7.4 Hz, 4H).

5-O-(2-Cyclopropylethyl)-myo-inositol (III_C-2): According to general debenzylation Procedure K, from 108 mg of (II_C-2), 15 mg of (III_C-2) were obtained (39% yield). 1H NMR (400 MHz, MeOH-d4) δ 3.88 (t, J=2.8 Hz, 1H), 3.80 (t, J=7.2 Hz, 2H), 3.61 (t, J=9.6 Hz, 2H), 3.29 (dd, J=9.6, 2.8 Hz, 3H), 2.91 (t, J=9.6 Hz, 1H), 1.47 (q, J=7.2 Hz, 2H), 0.80-0.62 (m, 1H), 0.41-0.30 (m, 2H), 0.07-0.10 (m, 2H).

5-O-(9-Methoxynonyl)-myo-inositol (III_C-3): According to general debenzylation Procedure K, from 127 mg of (II_C-3), 27 mg of (III_C-3) were obtained (50% yield). ¹H NMR (400 MHz, MeOH-d4) δ 3.96 (t, J=2.8 Hz, 1H), 3.80 (t, J=7.0 Hz, 2H), 3.67 (d, J=9.6 Hz, 2H), 3.40 (t, J=6.8 Hz, 2H), 3.35 (dd, J=9.6, 2.8 Hz, 2H), 2.97 (t, J=9.6 Hz, 1H), 1.64 (t, J=7.0 Hz, 3H), 1.57 (p, J=6.8 Hz, 3H), 1.35 (br, 10H).

rac-1-O-Propyl-myo-inositol (III_D-1): According to general debenzylation Procedure K, from 107 mg of (IX_D-1), 46 mg of (III_C-1) were obtained (95% yield). ¹H NMR (400 MHz, MeOH-d4) δ 4.04 (t, J=2.8 Hz, 1H), 3.59 (t, J=9.6 Hz, 1H), 3.54 (dt, J=9.2, 7.2 Hz, 1H), 3.53 (t, J=9.6 Hz, 1H), 3.38 (dt, J=9.2, 7.2 Hz, 1H), 3.24 (dd, J=9.6, 2.8 Hz, 1H), 3.09 (t, J=9.6 Hz, 1H), 3.01 (dd, J=9.6, 2.8 Hz, 1H), 1.55 (h, J=7.2 Hz, 2H), 0.85 (t, J=7.2 Hz, 3H).

rac-4-O-(6-Amino-6-oxohexyl)-myo-inositol (III_A-22): According to general hydrolysis Procedure S, from 72 mg of (II_A-21), 50 mg of (III_A-19) were obtained (80% yield). ¹H NMR (400 MHz, MeOH-d₄) δ 3.94 (t, J=2.5 Hz, 1H), 3.82-3.77 (m, 2H), 3.61 (t, J=9.5 Hz, 1H), 3.42-3.39 (m, 2H), 3.34-3.30 (m, 1H), 3.23-3.19 (m, 1H), 2.22 (t, J=7.4 Hz, 2H), 1.68-1.61 (m, 4H), 1.47-1.39 (m, 2H).

2-O-(5-(Benzyloxy)pentyl)-myo-inositol (III_B-7): According to general hydrolysis Procedure F, from 179 mg of (II_B-5), 75 mg of (III_B-7) were obtained (71% yield). HPLC-MS (Condition A): rt=2.83 min; m/z: 357 [M+1]⁺, 379 [M+23]⁺.

2-O-(6,6,6-Trifluorohexyl)-myo-inositol (III_B-8): According to general debenzylation Procedure K, from 85 mg of (VIII_B′-1), 54 mg of (III_B-8) were obtained (>99% yield). HPLC-MS (Condition A): rt=2.69 min; m/z: 319 [M+1]⁺.

2-O-(6-Amino-6-oxohexyl)-myo-inositol (III-B-9): According to general debenzylation Procedure K, from 75 mg of (VIII_B′-2), 45 mg of (III_B-9) were obtained (>99% yield). ¹H NMR (400 MHz, MeOH-d₄) δ 3.78 (t, J=6.4, 1H), 3.76 (t, J=6.4, 1H), 3.69 (q, J=3.0 Hz, 1H), 3.59 (td, J=10.0, 3.0 Hz, 2H), 3.39 (dt, J=10.0, 3.0 Hz, 2H), 3.14 (td, J=9.2, 3.6 Hz, 1H), 2.20 (t, J=7.6 Hz, 2H), 1.67-1.57 (m, 4H), 1.47-1.39 (m, 2H).

2-O-(2-Cyclopentylethyl)-myo-inositol (III_B-10): According to general debenzylation Procedure K, from 200 mg of (VIII_B′-3), 120 mg of (III_B-10) were obtained (>99% yield). HPLC-MS (Condition A): rt=2.69 min; m/z: 277 [M+1]⁺.

2-O-(2-Cyclopropylethyl)-myo-inositol (III_B-11): According to general debenzylation Procedure K, from 261 mg of (VIII_B′-4), 150 mg of (III_B-11) were obtained (>99% yield). ¹H NMR (400 MHz, MeOH-d₄) δ 3.84 (t, J=6.9 Hz, 2H), 3.70 (t, J=2.7 Hz, 1H), 3.58 (t, J=9.5 Hz, 2H), 3.37 (dd, J=9.5, 2.7 Hz, 2H), 3.12 (t, J=9.2 Hz, 1H), 1.50 (q, J=6.9 Hz, 2H), 0.81-0.69 (m, 1H), 0.43-0.39 (m, 2H), 0.07-0.03 (m, 2H).

2-O-(5-Aminopentyl)-myo-inositol (III_B-12): According to general hydrolysis Procedure F, from 105 mg of (II_B-6), 50 mg of (III_B-12) were obtained (93% yield). ¹H NMR (400 MHz, MeOH-d₄) δ 3.79 (t, J=6.2 Hz, 2H), 3.70 (t, J=2.7 Hz, 1H), 3.57 (t, J=9.2 Hz, 2H), 3.40 (dd, J=10.0, 2.7 Hz, 2H), 3.15 (t, J=9.2 Hz, 1H), 2.93 (t, J=7.6 Hz, 2H), 1.74-1.61 (m, 4H), 1.53-1.45 (m, 2H).

2-O-Propargyl-myo-inositol (III_B-13): According to general hydrolysis Procedure F, from 183 mg of (II_B-4), 73 mg of (III_B-13) were obtained (86% yield). ¹H NMR (400 MHz, MeOH-d₄) δ 4.49 (d, J=2.6 Hz, 2H), 3.93 (t, J=2.8 Hz, 1H), 3.57 (t, J=9.6 Hz, 2H), 3.40 (dd, J=10.0, 2.8 Hz, 2H), 3.13 (t, J=9.2 Hz, 1H), 2.81 (t, J=2.4 Hz, 1H).

2-O-(5-(4-(Methoxymethyl)-1H-1,2,3-triazol-1-yl)pentyl)-myo-inositol (III_B-14): According to general hydrolysis Procedure F, from 142 mg of (II_B-7), 65 mg of (III_B-14) were obtained (77% yield). HPLC-MS (Condition A): rt=1.28 min; m/z: 362 [M+1]⁺.

2-O-(5-Acetamidopentyl)-myo-inositol (III_B-15): According to general Amide formation Procedure Q, from 46 mg of (II_B-12) and 30 mg (1 eq.) of 2,5-dioxopyrrolidin-1-yl acetate, 30 mg of (III_B-15) were obtained (56% yield). HPLC-MS (Condition A): rt=1.06 min; m/z: 308 [M+1]⁺.

2-O-((1-(2-(Metoxycarbonyl)ethyl)-1H-1,2,3-triazol-4-yl)methyl)-myo-inositol (III-B-16): According to general click reaction Procedure L, from 109 mg of (III_B-13) and 71 mg (1.1 eq.) of (38), 118 mg of (III_B-16) were obtained (68%). HPLC-MS (Condition A): rt=0.97 min; m/z: 348 [M+1]⁺.

5-O-(5-Methoxypentyl)-myo-inositol (III_C-4): According to general debenzylation Procedure K, from 46 mg of (II_C-4), 17 mg of (III_C-4) were obtained (>99% yield). ¹H NMR (400 MHz, MeOH-d₄) δ 3.94 (t, J=2.8 Hz, 1H), 3.79 (t, J=6.7 Hz, 2H), 3.66 (t, J=9.5 Hz, 2H), 3.40 (t, J=6.5 Hz, 2H), 3.36-3.32 (dd, J=10.0, 2.8 Hz, 2H), 3.32 (s, 3H), 2.95 (t, J=9.2 Hz, 1H), 1.68-1.56 (m, 4H), 1.47-1.40 (m, 2H).

5-O-(6,6,6-Trifluorohexyl)-myo-inositol (III_C-5): According to general debenzylation Procedure K, from 160 mg of (II_C-5), 29 mg of (III_C-5) were obtained (43% yield). ¹H NMR (400 MHz, MeOH-d₄) δ 3.94 (t, J=2.8 Hz, 1H), 3.79 (t, J=6.6 Hz, 2H), 3.66 (t, J=9.6 Hz, 2H), 3.34 (dd, J=10.0, 2.8 Hz, 2H), 2.95 (t, J=9.2 Hz, 1H), 2.21-2.09 (m, 2H), 1.68-1.56 (m, 4H), 1.52-1.46 (m, 2H).

5-O-(5-(4-(Methoxymethyl)-1H-1,2,3-triazol-1-yl)pentyl)-myo-inositol (III_C-6): According to general debenzylation Procedure K, from 102 mg of (II_C-6), 15 mg of (III_C-6) were obtained (33% yield). ¹H NMR (400 MHz, MeOH-d₄) δ 7.97 (s, 1H), 4.53 (s, 2H), 4.42 (t, J=7.1 Hz, 2H), 3.94 (t, J=2.8 Hz, 1H), 3.78 (t, J=6.6 Hz, 2H), 3.65 (t, J=9.5 Hz, 2H), 3.37 (s, 3H), 3.33 (dd, J 10.0. 2.8 Hz, 2H), 2.94 (t, J=9.2 Hz, 1H), 1.95 (p, J=7.1 Hz, 2H), 1.66 (p, J=6.6 Hz, 2H), 1.45-1.38 (m, 2H).

rac-1-O-(9-Methoxynonyl)-myo-inositol (III_D-2): According to general debenzylation Procedure K, from 196 mg of (IX_D-2), 109 mg of (III_D-2) were obtained (>99% yield). HPLC-MS (Condition A): rt=2.89 min; m/z: 337 [M+1]⁺.

rac-1-O-(2-Cyclopropylethyl)-myo-inositol (III_D-3): According to general debenzylation Procedure K, from 181 mg of (IX_D-3), 87 mg of (III_D-3) were obtained (>99% yield). HPLC-MS (Condition A): rt=1.06 min; m/z: 249 [M+1]⁺.

rac-1-O-(5-Acetamidopentyl)-myo-inositol (III_D-4): According to general debenzylation Procedure K, from 107 mg of (IX_D-4), 57 mg of (III_D-4) were obtained (>99% yield). ¹H NMR (400 MHz, MeOH-d₄) δ 4.13 (s, 1H), 3.70-3.60 (m, 3H), 3.51 (q, J=7.1 Hz, 1H), 3.35-3.28 (m, 2H), 3-20-3.16 (m, 3H), 3.10 (d, J=9.5 Hz, 1H), 1.95 (s, 3H), 1.69-1.60 (m, 2H), 1.58-1.49 (m, 2H), 1.47-1.39 (m, 2H).

rac-1-O-(6,6,6-Trifluorohexyl)-myo-inositol (III_D-5): According to general debenzylation Procedure K, from 102 mg of (IX_D-5), 55 mg of (III_D-5) were obtained (>99% yield). HPLC-MS (Condition A): rt=2.72 min; m/z: 319 [M+1]⁺, 341 [M+23]⁺.

rac-1-O-(2-Cyclopentylethyl)-myo-inositol (III_D-6): According to general debenzylation Procedure K, from 119 mg of (IX_D-6), 76 mg of (III_D-6) were obtained (64% yield). HPLC-MS (Condition A): rt=2.72 min; m/z: 277 [M+1]⁺, 299 [M+23]⁺.

rac-1-O-((1-(2-(Metoxycarbonyl)ethyl)-1H-1,2,3-triazol-4-yl)methyl)-myo-inositol (III_D-7): According to general debenzylation Procedure K, from 248 mg of (IX_D-10), 130 mg of (III_D-7) were obtained (93% yield). HPLC-MS (Condition A): rt=0.99 min; m/z: 348 [M+1]⁺.

rac-1-O-(5-(4-(Methoxymethyl)-1H-1,2,3-triazol-1-yl)pentyl)-myo-inositol (III_D-8): According to general debenzylation Procedure K, from 250 mg of (IX_D-8), 143 mg of (III_D-8) were obtained (>99% yield). ¹H NMR (400 MHz, MeOH-d4) δ 8.04 (s, 1H), 4.57 (s, 2H), 4.46 (t, J=7.0 Hz, 2H), 4.17 (t, J=2.8 Hz, 1H), 3.74-3.65 (m, 3H), 3.54 (dt, J=9.4, 6.5 Hz, 1H), 3.41 (s, 3H), 3.40-3.37 (m 1H), 3.24 (t, J=9.2 Hz, 1H), 3.15 (dd, J=10.0, 2.8 Hz, 1H), 1.98 (p, J=7.2 Hz, 2H), 1.69 (p, J=6.9 Hz, 2H), 1.48-1.41 (m, 2H).

### C.3. Intermediates IV_A, IV_B, IV_C and IV_D

rac-1,2,3,5,6-pentakis-O-(3-Oxido-1,5-dihydrobenzo[e][1,3,2]dioxaphosphepin-3-yl)-4-O-pentyl-myo-inositol (IV_A-1): According to general phosphorylation Procedure H, from 76 mg of (III_A-1), 57.6 mg of (IV_A-1) were obtained (16% yield). HPLC-MS (Condition A): rt=4.15 min; m/z: 1161 [M+1]⁺.

rac-4-O-(5-(Benzyloxy)pentyl)-1,2,3,5,6-pentakis-O-(3-oxido-1,5-dihydrobenzo[e][1,3,2]dioxaphosphepin-3-yl)-myo-inositol (IV_A-2): According to general phosphorylation Procedure H, from 20 mg of (III_A-2), 30 mg of (IV_A-2) were obtained (42% yield). HPLC-MS (Condition B): rt=3.24 min; m/z: 1267 [M+1]⁺.

rac-4-O-(5-Methoxypentyl)-1,2,3,5,6-pentakis-O-(3-oxido-1,5-dihydrobenzo[e][1,3,2]dioxaphosphepin-3-yl)-myo-inositol (IV_A-3): According to general phosphorylation Procedure H, from 45 mg of (III_A-3), 98 mg of (IV_A-3) were obtained (51% yield). HPLC-MS (Condition B): rt=2.95 min; m/z: 1191 [M+1]⁺.

rac-4-O-(5-Methoxycarbonylpentyl)-1,2,3,5,6-pentakis-O-(3-oxido-1,5-dihydrobenzo[e][1,3,2]dioxaphosphepin-3-yl)myo-inositol (IV_A-4): According to general phosphorylation Procedure G, from 42 mg of (III_A-4), 23 mg of (IV_A-4) were obtained (14% yield). HPLC-MS (Condition B): rt=2.99 min; m/z: 1219 [M+1]⁺.

rac-4-O-(2-(4-Acetylpiperazin-1-yl)ethyl)-1,2,3,5,6-pentakis-O-(3-oxido-1,5-dihydrobenzo[e][1,3,2]dioxaphosphepin-3-yl)-myo-inositol (IV_A-5): According to general phosphorylation Procedure H, from 24 mg of (III_A-5), 29 mg of (IV_A-5) were obtained (32% yield). HPLC-MS (Condition B): rt=2.37 min.; m/z: 1245[M+1]⁺.

1,3,4,5,6-pentakis-O-(3-Oxido-1,5-dihydrobenzo[e][1,3,2]dioxaphosphepin-3-yl)-2-O-pentyl-myo-inositol (IV_B-1): According to general phosphorylation Procedure H, from 150 mg of (III_B-1), 399 mg of (IV_B-1) were obtained (57% yield). HPLC-MS (Condition A): rt=4.15 min; m/z: 1161 [M+1]⁺.

2-O-(5-Methoxypentyl)-1,3,4,5,6-pentakis-O-(3-oxido-1,5-dihydrobenzo[e][1,3,2]dioxaphosphepin-3-yl)-myo-inositol (IV_B-2): According to general phosphorylation Procedure H, from 84 mg of (III_B-2), 71 mg of (IV_B-2) were obtained (19.9% yield). HPLC-MS (Condition A): rt=3.99 min; m/z: 1191.6 [M+1]⁺.

2-O-(9-Methoxynonyl)-1,3,4,5,6-pentakis-O-(3-oxido-1,5-dihydrobenzo[e][1,3,2]dioxaphosphepin-3-yl)-myo-inositol (IV_B-3): According to general phosphorylation Procedure H, from 240 mg of (III_B-3), 110 mg of (IV_B-3) were obtained (12.4% yield). HPLC-MS (Condition B): rt=3.95 min; m/z: 1247 [M+1]⁺, 1264 [M+23]⁺.

2-O-(19-Methoxynonadecyl)-1,3,4,5,6-pentakis-O-(3-oxido-1,5-dihydrobenzo[e][1,3,2]dioxaphosphepin-3-yl)-myo-inositol (IV_B-4): According to general phosphorylation Procedure H, from 200 mg of (III_B-4), 80 mg of (IV_B-4) were obtained (14% yield). HPLC-MS (Condition B): rt=5.17 min; m/z: 1387 [M+1]⁺.

2-O-(29-Methoxynonacosyl)-1,3,4,5,6-pentakis-O-(3-oxido-1,5-dihydrobenzo[e][1,3,2]dioxaphosphepin-3-yl)-myo-inositol (IV_B-5): According to general phosphorylation Procedure H, from 75 mg of (III_B-5), 20 mg of (IV_B-5) were obtained (11% yield). ³¹P NMR (162 MHz, Chloroform-d) δ −1.15, −4.17, −4.76.

rac-1,2,3,5,6-pentakis-O-(3-Oxido-1,5-dihydrobenzo[e][1,3,2]dioxaphosphepin-3-yl)-4-O-(3-(p-Tolyl)propyl)-myo-inositol (IV_A-6): According to general phosphorylation Procedure H, from 50 mg of (III_A-6), 44.6 mg of (IV_A-6) were obtained (23% yield). ³¹P NMR (162 MHz, Chloroform-d) δ −0.71, −1.32, −1.89, −3.00, −4.16. HPLC-MS (Condition A): rt=4.46 min; m/z: 1223 [M+1]⁺.

rac-4-O-(4-Methylpentyl)-1,2,3,5,6-pentakis-O-(3-oxido-1,5-dihydrobenzo[e][1,3,2]dioxaphosphepin-3-yl)-myo-inositol (IV_A-7): According to general phosphorylation Procedure H, from 50 mg of (III_A-7), 45 mg of (IV_A-7) were obtained (20% yield). ³¹P NMR (162 MHz, Chloroform-d) δ −0.50, −1.20, −1.96, −3.05, −4.16. HPLC-MS (Condition A): rt=4.44 min; m/z: 1175 [M+1]⁺.

rac-1,2,3,5,6-pentakis-O-(3-oxido-1,5-dihydrobenzo[e][1,3,2]dioxaphosphepin-3-yl)-4-O-(6,6,6-Trifluorohexyl)-myo-inositol (IV_A-8): According to general phosphorylation Procedure H, from 50 mg of (III_A-8), 48 mg of (IV_A-8) were obtained (25% yield). ³¹P NMR (162 MHz, Chloroform-d) −0.67, −1.28, −1.85, −2.99, −4.10. HPLC-MS (Condition A): rt=4.31 min; m/z: 1229 [M+1]⁺.

rac-4-O-(3-(4-Methoxyphenyl)propyl)-1,2,3,5,6-pentakis-O-(3-oxido-1,5-dihydrobenzo[e][1,3,2]dioxaphosphepin-3-yl)-myo-inositol (IV_A-9): According to general phosphorylation Procedure H, from 50 mg of (III_A-9), 48 mg of (IV_A-9) were obtained (25% yield). ³¹P NMR (162 MHz, Chloroform-d) δ −0.74, −1.33, −1.85, −3.00, −3.00, −4.15. HPLC-MS (Condition A): rt=4.29 min; m/z: 1240 [M+1]⁺.

rac-1,2,3,5,6-pentakis-O-(3-Oxido-1,5-dihydrobenzo[e][1,3,2]dioxaphosphepin-3-yl)-4-O-(3-(3-(trifluoromethyl)phenyl)propyl)-myo-inositol (IV_A-10): According to general phosphorylation Procedure H, from 50 mg of (III_A-10), 35 mg of (IV_A-10) were obtained (20% yield). ³¹P NMR (162 MHz, Chloroform-d) δ −0.52, −1.24, −1.84, −3.01, −4.11. HPLC-MS (Condition A): rt=4.51 min; 1277 m/z: [M+1]⁺.

rac-4-O-(2-Cyclopentylethyl)-1,2,3,5,6-pentakis-O-(3-oxido-1,5-dihydrobenzo[e][1,3,2]dioxaphosphepin-3-yl)-myo-inositol (IV_A-11): According to general phosphorylation Procedure H, from 45 mg of (III_A-11), 33 mg of (IV_A-11) were obtained (17% yield). ³¹P NMR (162 MHz, Chloroform-d) −0.59, −1.18, −1.93, −3.03, −4.15. HPLC-MS (Condition A): rt=4.46 min; m/z: 1187 [M+1]⁺.

rac-4-O-(2-Cyclopropylethyl)-1,2,3,5,6-pentakis-O-(3-oxido-1,5-dihydrobenzo[e][1,3,2]dioxaphosphepin-3-yl)-myo-inositol (IV_A-12): According to general phosphorylation Procedure H, from 52 mg of (III_A-12), 47 mg of (IV_A-12) were obtained (19% yield). ³¹P NMR (162 MHz, Chloroform-d) δ −0.68, −1.24, −1.90, −3.00, −4.15. HPLC-MS (Condition A): rt=4.15 min; m/z: 1159 [M+1]⁺.

rac-1,2,3,5,6-pentakis-O-(3-Oxido-1,5-dihydrobenzo[e][1,3,2]dioxaphosphepin-3-yl)-4-O-(5-(1H-Pyrazol-1-yl)pentyl)-myo-inositol (IV_A-13): According to general phosphorylation Procedure H, from 50 mg of (III_A-13), 100 mg of (IV_A-13) were obtained (54% yield). ³¹P NMR (162 MHz, Chloroform-d) δ −0.72, −1.39, −1.88, −3.00, −4.13. HPLC-MS (Condition A): rt=4.08 min; m/z: 1227 [M+1]⁺.

rac-4-O-(5-Acetamidopentyl)-1,2,3,5,6-pentakis-O-(3-oxido-1,5-dihydrobenzo[e][1,3,2]dioxaphosphepin-3-yl)-myo-inositol (IV_A-14): According to general phosphorylation Procedure H, from 35 mg of (III_A-14), 14 mg of (IV_A-14) were obtained (10% yield). ³¹P NMR (162 MHz, Chloroform-d) δ −0.75, −1.63, −1.78, −2.98, −4.04. HPLC-MS (Condition A): rt=3.82 min; m/z: 1218 [M+1]⁺.

rac-4-O-(10-Methoxycarbonyldecyl)-1,2,3,5,6-pentakis-O-(3-oxido-1,5-dihydrobenzo[e][1,3,2]dioxaphosphepin-3-yl)-myo-inositol (IV_A-15): According to general phosphorylation Procedure H, from 30 mg of (III_A-15), 7.5 mg of (IV_A-15) were obtained (7% yield). ³¹P NMR (162 MHz, Chloroform-d) −0.51, −1.17, −1.92, −3.07, −4.18. HPLC-MS (Condition A): rt=4.65 min; m/z: 1289 [M+1]⁺.

rac-4-O-(10-(Benzyloxy)decyl)-1,2,3,5,6-pentakis-O-(3-oxido-1,5-dihydrobenzo[e][1,3,2]dioxaphosphepin-3-yl)-myo-inositol (IV_A-16): According to general phosphorylation Procedure H, from 19 mg of (III_A-16), 13 mg of (IV_A-16) were obtained (22% yield). ³¹P NMR (162 MHz, Chloroform-d) δ −0.50, −1.14, −1.93, −3.07, −4.18. HPLC-MS (Condition A): rt=5.11 min; m/z: 1337 [M+1]⁺.

rac-4-O-Methyl-1,2,3,5,6-pentakis-O-(3-oxido-1,5-dihydrobenzo[e][1,3,2]dioxaphosphepin-3-yl)-myo-inositol (IV_A-17): According to general phosphorylation Procedure H, from 27 mg of (III_A-17), 10 mg of (IV_A-17) were obtained (6% yield). ³¹P NMR (162 MHz, Chloroform-d) δ −0.83, −1.43, −1.80, −3.17, −4.20. HPLC-MS (Condition A): rt=3.96 min; m/z: 1105 [M+1]⁺.

rac-4-O-(7-Methoxyheptyl)-1,2,3,5,6-pentakis-O-(3-oxido-1,5-dihydrobenzo[e][1,3,2]dioxaphosphepin-3-yl)-myo-inositol (IV_A-18): According to general phosphorylation Procedure H, from 50 mg of (III_A-18), 32 mg of (IV_A-18) were obtained (16% yield). ³¹P NMR (162 MHz, Chloroform-d) δ −0.54, −1.18, −1.92, −3.06, −4.17. HPLC-MS (Condition A): rt=4.25 min; m/z: 1219 [M+1]⁺.

rac-1,2,3,5,6-pentakis-O-(3-Oxido-1,5-dihydrobenzo[e][1,3,2]dioxaphosphepin-3-yl)-4-O-Propyl-myo-inositol (IV_A-19): According to general phosphorylation Procedure H, from 66 mg of (III_A-19), 20 mg of (IV_A-19) were obtained (6% yield). ³¹P NMR (162 MHz, Chloroform-d) −0.59, −1.24, −1.92, −3.01, −4.14. HPLC-MS (Condition A): rt=3.98 min; m/z: 1133 [M+1]⁺.

rac-4-O-(3-(4-(2-(Benzyloxycarbonyl)ethyl)-1H-1,2,3-triazol-1-yl)propyl)-1,2,3,5,6-pentakis-O-(3-oxido-1,5-dihydrobenzo[e][1,3,2]dioxaphosphepin-3-yl)-myo-inositol (IV_A-20): According to general phosphorylation Procedure H, from 34 mg of (III_A-20), 14 mg of (IV_A-20) were obtained (14% yield). ³¹P NMR (162 MHz, Chloroform-d) δ −0.63, −1.61 (d, J=6.2 Hz), −2.97, −3.98. HPLC-MS (Condition A): rt=4.16 min; m/z: 1363 [M+1]⁺.

rac-4-O-(6-(4-(Methoxycarbonyl)-1H-1,2,3-triazol-1-yl)hexyl)-1,2,3,5,6-pentakis-O-(3-oxido-1,5-dihydrobenzo[e][1,3,2]dioxaphosphepin-3-yl)-myo-inositol (IV_A-21): According to general phosphorylation Procedure H, from 40 mg of (III_A-21), 15 mg of (IV_A-21) were obtained (11% yield). ³¹P NMR (162 MHz, Chloroform-d) δ −0.95, −1.45, −1.78, −3.04, −4.12. HPLC-MS (Condition A): rt=4.00 min; m/z: 1300[M+1]⁺.

1,2,3,4,6-pentakis-O-(3-Oxido-1,5-dihydrobenzo[e][1,3,2]dioxaphosphepin-3-yl)-5-O-Propyl-myo-inositol (IV_C-1): According to general phosphorylation Procedure H, from 15 mg of (III_C-1), 10 mg of (IV_C-1) were obtained (13% yield). ³¹P NMR (162 MHz, Chloroform-d) δ −0.16, −3.40, −3.90. HPLC-MS (Condition A): rt=4.04 min; m/z: 1133 [M+1]⁺.

5-O-(2-Cyclopropylethyl)-1,2,3,4,6-pentakis-O-(3-oxido-1,5-dihydrobenzo[e][1,3,2]dioxaphosphepin-3-yl)-myo-inositol (IV_C-2): According to general phosphorylation Procedure H, from 15 mg of (III_C-2), 10 mg of (IV_C-2) were obtained (14% yield). ³¹P NMR (162 MHz, Chloroform-d) δ −0.21, −3.36, −3.90. HPLC-MS (Condition A): rt=4.17 min; m/z: 1160 [M+1]⁺.

5-O-(9-Methoxynonyl)-1,2,3,4,6-pentakis-O-(3-oxido-1,5-dihydrobenzo[e][1,3,2]dioxaphosphepin-3-yl)-myo-inositol (IV_C-3): According to general phosphorylation Procedure H, from 27 mg of (III_C-3), 15 mg of (IV_C-3) were obtained (15% yield). ³¹P NMR (162 MHz, Chloroform-d) δ −0.07, −3.35, −3.92. HPLC-MS (Condition A): rt=4.55 min; m/z: 1248 [M+1]⁺.

rac-2,3,4,5,6-pentakis-O-(3-Oxido-1,5-dihydrobenzo[e][1,3,2]dioxaphosphepin-3-yl)-1-O-propyl-myo-inositol (IV_D-1): According to general phosphorylation Procedure H, from 61 mg of (III_D-1), 53 mg of (IV_D-1) were obtained (17% yield). ³¹P NMR (162 MHz, Chloroform-d) δ −1.76, −2.59, −3.97, −4.47, −4.69. HPLC-MS (Condition A): rt=4.10 min; m/z: 1133 [M+1]⁺.

rac-4-O-(6-Amino-6-oxohexyl)-1,2,3,5,6-pentakis-O-(3-oxido-1,5-dihydrobenzo[e][1,3,2]dioxaphosphepin-3-yl)-myo-inositol (IV_A-22): According to general phosphorylation Procedure H, from 130 mg of (III_A-22), 6 mg of (IV_A-22) were obtained (1% yield). ³¹P NMR (162 MHz, Chloroform-d) δ −1.64, −1.81, −2.31, −2.89, −4.09. HPLC-MS (Condition A): rt=3.44 min; m/z: 1205 [M+1]⁺.

1,3,4,5,6-pentakis-O-(3-Oxido-1,5-dihydrobenzo[e][1,3,2]dioxaphosphepin-3-yl)-2-O-propargyl-myo-inositol (IV_B-6): According to general phosphorylation Procedure H, from 54 mg of (III_B-13), 10 mg of (IV_B-6) were obtained (3.6% yield). ³¹P NMR (162 MHz, Chloroform-d) δ −2.03, −4.28, −4.89. HPLC-MS (Condition A): rt=3.98 min; m/z: 1129 [M+1]⁺.

2-O-(5-(Benzyloxy)pentyl)-1,3,4,5,6-pentakis-O-(3-oxido-1,5-dihydrobenzo[e][1,3,2]dioxaphosphepin-3-yl)-myo-inositol (IV_B-7): According to general phosphorylation Procedure H, from 67 mg of (III_B-7), 75 mg of (IV_B-7) were obtained (32% yield). ³¹P NMR (162 MHz, Chloroform-d) δ −1.06, −4.00, −4.63. HPLC-MS (Condition A): rt=4.43 min; m/z: 1267 [M+1]⁺.

2-O-(6,6,6-Trifluorohexyl)-1,3,4,5,6-pentakis-O-(3-oxido-1,5-dihydrobenzo[e][1,3,2]dioxaphosphepin-3-yl)-myo-inositol (IV_B-8): According to general phosphorylation Procedure R, from 54 mg of (III_B-8), 56 mg of (IV_B-8) were obtained (27% yield). ³¹P NMR (162 MHz, Chloroform-d) δ 6-0.76, −3.76, −4.57. HPLC-MS (Condition A): rt=4.37 min; m/z: 1229 [M+1]⁺.

2-O-(6-Amino-6-oxohexyl)-1,3,4,5,6-pentakis-O-(3-oxido-1,5-dihydrobenzo[e][1,3,2]dioxaphosphepin-3-yl)-myo-inositol (IV_B-9): According to general phosphorylation Procedure R, from 46 mg of (III_B-9), 23 mg of (IV_B-9) were obtained (14% yield). ³¹P NMR (162 MHz, Chloroform-d) δ −0.71, −4.15, −4.34. HPLC-MS (Condition A): rt=3.77 min; m/z: 1204 [M+1]⁺.

2-O-(2-Cyclopentylethyl)-1,3,4,5,6-pentakis-O-(3-oxido-1,5-dihydrobenzo[e][1,3,2]dioxaphosphepin-3-yl)-myo-inositol (IV_B-10): According to general phosphorylation Procedure R, from 121 mg of (III_B-10), 124 mg of (IV_B-10) were obtained (24% yield). ³¹P NMR (162 MHz, Chloroform-d) δ −1.09, −4.00, −4.64. HPLC-MS (Condition A): rt=4.40 min; m/z: 1187 [M+1]⁺.

2-O-(2-Cyclopropylethyl)-1,3,4,5,6-pentakis-O-(3-oxido-1,5-dihydrobenzo[e][1,3,2]dioxaphosphepin-3-yl)-myo-inositol (IV_B-11): According to general phosphorylation Procedure H, from 62 mg of (III_B-11), 85 mg of (IV_B-11) were obtained (29% yield). ³¹P NMR (162 MHz, Chloroform-d) δ −1.30, −3.99, −4.70. HPLC-MS (Condition A): rt=4.15 min; m/z: 1159 [M+1]⁺.

2-O-(5-(4-(Methoxymethyl)-1H-1,2,3-triazol-1-yl)pentyl)-1,3,4,5,6-pentakis-O-(3-oxido-1,5-dihydrobenzo[e][1,3,2]dioxaphosphepin-3-yl)-myo-inositol (IV_B-12): According to general phosphorylation Procedure G, from 65 mg of (III_B-14), 25 mg of (IV_B-12) were obtained (11% yield). ³¹P NMR (162 MHz, Chloroform-d) δ −0.64, −3.84, −4.44. HPLC-MS (Condition A): rt=3.92 min; m/z: 1272 [M+1]⁺.

2-O-(5-Acetamidopentyl)-1,3,4,5,6-pentakis-O-(3-oxido-1,5-dihydrobenzo[e][1,3,2]dioxaphosphepin-3-yl)-myo-inositol (IV_B-13): According to general phosphorylation Procedure H, from 30 mg of (III_B-15), 10 mg of (IV_B-13) were obtained (8% yield). ³¹P NMR (162 MHz, Chloroform-d) δ −0.61, −4.30 (d, J=3.6 Hz). HPLC-MS (Condition A): rt=3.73 min; m/z: 1218 [M+1]⁺.

2-O-((1-(2-(Metoxycarbonyl)ethyl)-1H-1,2,3-triazol-4-yl)methyl)-1,3,4,5,6-pentakis-O-(3-oxido-1,5-dihydrobenzo[e][1,3,2]dioxaphosphepin-3-yl)-myo-inositol (IV_B-14): According to general phosphorylation Procedure G, from 118 mg of (III_B-16), 101 mg of (IV_B-14) were obtained (24% yield). ³¹P NMR (162 MHz, Chloroform-d) δ −1.44, −4.02, −4.58. HPLC-MS (Condition A): rt=3.84 min; m/z: 1258 [M+1]⁺.

5-O-(5-Methoxypentyl)-1,2,3,4,6-pentakis-O-(3-oxido-1,5-dihydrobenzo[e][1,3,2]dioxaphosphepin-3-yl)-myo-inositol (IV_C-4): According to general phosphorylation Procedure H, from 23 mg of (III_C-4), 6 mg of (IV_C-4) were obtained (6% yield). ³¹P NMR (162 MHz, Chloroform-d) δ −0.06, −3.39, −3.90. HPLC-MS (Condition A): rt=4.03 min; m/z: 1192 [M+1]⁺.

5-O-(6,6,6-Trifluorohexyl)-1,2,3,4,6-pentakis-O-(3-oxido-1,5-dihydrobenzo[e][1,3,2]dioxaphosphepin-3-yl)-myo-inositol (IV_C-5): According to general phosphorylation Procedure H, from 28 mg of (III_C-5), 10 mg of (IV_C-5) were obtained (9% yield). ³¹P NMR (162 MHz, Chloroform-d) δ −0.11, −3.21, −3.83. HPLC-MS (Condition A): rt=4.34 min; m/z: 1230 [M+1]⁺.

5-O-(5-(4-(Methoxymethyl)-1H-1,2,3-triazol-1-yl)pentyl)-1,2,3,4,6-pentakis-O-(3-oxido-1,5-dihydrobenzo[e][1,3,2]dioxaphosphepin-3-yl)-myo-inositol (IV_C-6): According to general phosphorylation Procedure H, from 15 mg of (III_C-6), 7 mg of (IV_C-6) were obtained (13% yield). ³¹P NMR (162 MHz, Chloroform-d) δ −0.50, −2.93, −3.74. HPLC-MS (Condition A): rt=3.84 min; m/z: 1273 [M+1]⁺.

rac-1-O-(9-Methoxynonyl)-2,3,4,5,6-pentakis-O-(3-oxido-1,5-dihydrobenzo[e][1,3,2]dioxaphosphepin-3-yl)-myo-inositol (IV_D-2): According to general phosphorylation Procedure H, from 113 mg of (III_D-2), 100 mg of (IV_D-2) were obtained (24% yield). ³¹P NMR (162 MHz, Chloroform-d) δ −1.97, −2.60, −4.01, −4.49, −4.61. HPLC-MS (Condition A): rt=4.40 min; m/z: 1248 [M+1]⁺.

rac-1-O-(2-Cyclopropylethyl)-2,3,4,5,6-pentakis-O-(3-oxido-1,5-dihydrobenzo[e][1,3,2]dioxaphosphepin-3-yl)-myo-inositol (IV_D-3): According to general phosphorylation Procedure H, from 96 mg of (III_D-3), 90 mg of (IV_D-3) were obtained (20% yield). ³¹P NMR (162 MHz, Chloroform-d) δ −2.28, −2.56, −4.03, −4.49, −4.58. HPLC-MS (Condition A): rt=4.08 min; m/z: 1160 [M+1]⁺.

rac-1-O-(5-Acetamidopentyl)-2,3,4,5,6-pentakis-O-(3-oxido-1,5-dihydrobenzo[e][1,3,2]dioxaphosphepin-3-yl)-myo-inositol (IV_D-4): According to general phosphorylation Procedure G, from 59 mg of (III_D-4), 10 mg of (IV_D-4) were obtained (4% yield). ³¹P NMR (162 MHz, Chloroform-d) δ −2.18, −2.69, −3.76, −4.30, −4.54. HPLC-MS (Condition A): rt=3.71 min; m/z: 1219 [M+1]⁺.

rac-1-O-(6,6,6-Trifluorohexyl)-2,3,4,5,6-pentakis-O-(3-oxido-1,5-dihydrobenzo[e][1,3,2]dioxaphosphepin-3-yl)-myo-inositol (IV_D-5): According to general phosphorylation Procedure H, from 64 mg of (III_D-5), 25 mg of (IV_D-5) were obtained (10% yield). ³¹P NMR (162 MHz, Chloroform-d) δ −1.90, −2.58, −3.97, −4.46, −4.64. HPLC-MS (Condition A): rt=4.28 min; m/z: 1230 [M+1]⁺.

rac-1-O-(2-Cyclopentylethyl)-2,3,4,5,6-pentakis-O-(3-oxido-1,5-dihydrobenzo[e][1,3,2]dioxaphosphepin-3-yl)-myo-inositol (IV_D-6): According to general phosphorylation Procedure H, from 71 mg of (III_D-6), 25 mg of (IV_D-6) were obtained (8% yield). ³¹P NMR (162 MHz, Chloroform-d) δ −1.94, −2.49, −4.12, −4.50, −4.71. HPLC-MS (Condition A): rt=4.35 min; m/z: 1188 [M+1]⁺.

rac-1-O-((1-(2-(Metoxycarbonyl)ethyl)-1H-1,2,3-triazol-4-yl)methyl)-2,3,4,5,6-pentakis-O-(3-oxido-1,5-dihydrobenzo[e][1,3,2]dioxaphosphepin-3-yl)-myo-inositol (IV_D-7): According to general phosphorylation Procedure G, from 130 mg of (III_D-7), 104 mg of (IV_D-7) were obtained (22% yield). ³¹P NMR (162 MHz, Chloroform-d) δ −2.38, −2.62, −3.63, −4.36, −4.50. HPLC-MS (Condition A): rt=3.89 min; m/z: 1259 [M+1]⁺.

rac-1-O-(5-(4-(Methoxymethyl)-1H-1,2,3-triazol-1-yl)pentyl)-2,3,4,5,6-pentakis-O-(3-oxido-1,5-dihydrobenzo[e][1,3,2]dioxaphosphepin-3-yl)-myo-inositol (IV_D-8): According to general phosphorylation Procedure H, from 46 mg of (III_D-8), 10 mg of (IV_D-8) were obtained (6% yield). ³¹P NMR (162 MHz, Chloroform-d) δ −2.06, −2.65, −3.84, −4.36, −4.59. HPLC-MS (Condition A): rt=3.86 min; m/z: 1273 [M+1]⁺.

### C.4. Intermediates V_A, V_B, V_C and V_D

rac-4-O-(3-Azidopropyl)-2-O-tert-butyldimethylsilyl-1,3,5-O-methylidyne-myo-inositol (V_A-1): According to general alkylation Procedure A, from 613 mg (2.1 eq.) of (18), 85 mg of (V_A-1) were obtained (19%). HPLC-MS (Condition B): rt=3.15 min; m/z: 388 [M+1]⁺.

rac-4-O-(6-Azidohexyl)-2-O-tert-butyldimethylsilyl-1,3,5-O-methylidyne-myo-inositol (V_A-2): According to general alkylation Procedure A, from 391 mg (2 eq.) of (22), 36 mg of (V_A-2) were obtained (13%). HPLC-MS (Condition B): rt=4.83 min; m/z: 430 [M+1]⁺.

2-O-(5-Azidopentyl)-4,6-bis-O-(4-methoxybenzyl)-1,3,5-O-methylidyne-myo-inositol (V_B-1): According to general alkylation Procedure A, from 236 mg of (3) and 311 mg (2 eq) of (37), 225 mg of (V_B-1) were obtained (76% yield). HPLC-MS (Condition A): rt=4.55 min; m/z: 542 [M+1]⁺.

5-O-(5-Azidopentyl)-1,2,3,4,6-penta-O-benzyl-myo-inositol (V_C-1): According to general alkylation Procedure A, from 200 mg of (33) and 180 mg (2 eq) of (37), 218 mg of (V_C-1) were obtained (93% yield). HPLC-MS (Condition A): rt=6.30 min; m/z: 765 [M+23]⁺.

rac-1-O-(5-Azidopentyl)-2,4,6-tri-O-benzyl-3,5-O-ethylidene-myo-inositol (V_D-1): According to general alkylation Procedure B, from 285 mg of (34) and 254 mg (1.5 eq) of (37), 351 mg mg of (V_D-1) were obtained (>99% yield). HPLC-MS (Condition A): rt=5.55 min; m/z: 610 [M+23]⁺.

### C.5. Intermediates VI_A

rac-4-O-(3-Azidopropyl)-myo-inositol (VI_A-1): According to general Hydrolysis Procedure S, from 166 mg of (V_A-1), 110 mg of (VI_A-1) were obtained (97%). HPLC-MS (Condition A): rt=2.59 min; m/z: 265 [M+1]⁺.

rac-4-O-(6-Azidohexyl)-myo-inositol (VI_A-2): According to general Hydrolysis Procedure S, from 279 mg of (V_A-2), 160 mg of (VI_A-2) were obtained (81%). HPLC-MS (Condition A): rt=2.59 min; m/z: 306 [M+1]⁺.

### C.6. Intermediates VII_A

rac-4-O-(3-Azidopropyl)-1,2,3,5,6-pentakis-O-(3-oxido-1,5-dihydrobenzo[e][1,3,2]dioxaphosphepin-3-yl)-myo-inositol (VII_A-1): According to general phosphorylation Procedure H, from 55 mg of (VI_A-1), 15 mg of (VII_A-2) were obtained (6% yield). ³¹P NMR (162 MHz, Chloroform-d) δ −0.38, −1.11, −1.80, −3.06, −4.12. HPLC-MS (Condition A): rt=4.11 min; m/z: 1174 [M+1]⁺.

rac-4-O-(3-Azidopropyl)-1,2,3,5,6-pentakis-O-(3-oxido-1,5-dihydrobenzo[e][1,3,2]dioxaphosphepin-3-yl)-myo-inositol (VII_A-2): According to general phosphorylation Procedure H, from 40 mg of (VI_A-1), 44 mg of (VII_A-2) were obtained (28% yield). ³¹P NMR (162 MHz, Chloroform-d) δ −0.63, −1.25, −1.88, −3.02, −4.14. HPLC-MS (Condition A): rt=4.31 min; m/z: 1216 [M+1]⁺.

### C.7. Intermediates VIII_B and VIII-B′

2-O-(19-Methoxynonadecyl)-1,3,5-O-methylidyne-myo-inositol (VIII_B-1): According to general debenzylation Procedure K, from 504 mg of (II_B′-1), 337 mg of (VIII_B-1) were obtained (91% yield). ¹H NMR (400 MHz, Chloroform-d) δ 5.50 (d, J=1.3 Hz, 1H), 4.58 (t, J=3.9 Hz, 2H), 4.32 (br, 2H), 4.27 (br, 1H), 3.82 (br, 1H), 3.62 (t, J=6.8 Hz, 2H), 3.35 (t, J=6.8 Hz, 2H), 3.31 (s, 3H), 1.67 (p, J=7.2 Hz, 2H), 1.54 (p, J=7.2 Hz, 2H), 1.38-1.18 (s, 50H).

2-O-(29-Methoxynonacosyl)-1,3,5-O-methylidyne-myo-inositol (VIII_B-2): According to general debenzylation Procedure K, from 776 mg of (II_B′-2), 282 mg of (VIII_B-2) were obtained (46% yield). ¹H NMR (400 MHz, Chloroform-d) δ 5.49 (d, J=1.3 Hz, 1H), 4.57 (t, J=4.0 Hz, 2H), 4.32 (dt, J=4.7, 1.7 Hz, 2H), 4.26 (br, 1H), 3.82 (d, J=1.7 Hz, 1H), 3.61 (t, J=6.8 Hz, 2H), 3.35 (t, J=6.8 Hz, 2H), 3.31 (s, 3H), 1.65 (q, J=8.7, 7.0 Hz, 2H), 1.54 (p, J=6.8 Hz, 2H), 1.38-1.18 (s, 59H).

4,6-di-O-Benzyl-2-O-(6,6,6-trifluorohexyl)-myo-inositol (VIII B′-1): According to general hydrolysis Procedure S, from 90 mg of (II_B′-3), 85 mg of (VIII_B′-1) were obtained (96% yield). HPLC-MS (Condition A): rt=4.19 min; m/z: 521 [M+23]⁺.

2-O-(6-Amino-6-oxohexyl)-4,6-di-O-benzyl-myo-inositol (VIII B′-2): According to general hydrolysis Procedure S, from 70 mg of (II_B′-4), 68 mg of (VIII_B′-2) were obtained (>99% yield). HPLC-MS (Condition A): rt=3.19 min; m/z: 474 [M+1]⁺.

4,6-di-O-Benzyl-2-O-(2-cyclopentylethyl)-myo-inositol (VIII B′-3): According to general hydrolysis Procedure S, from 185 mg of (II_B′-5), 180 mg of (VIII_B′-3) were obtained (>99% yield). HPLC-MS (Condition A): rt=4.43 min; m/z: 457 [M+1]⁺, 479 [M+23]⁺.

4,6-di-O-Benzyl-2-O-(2-cyclopropylethyl)-myo-inositol (VIII B′-4): According to general hydrolysis Procedure S, from 237 mg of (II_B′-6), 230 mg of (VIII_B′-4) were obtained (>99% yield). HPLC-MS (Condition A): rt=4.43 min; m/z: 451 [M+23]⁺.

### C.8. Intermediates IX_D

rac-2,4,6-tri-O-Benzyl-1-O-propyl-myo-inositol (IX_D-1): According to general hydrolysis Procedure S, from 68 mg of (II_D-1), 63 mg of (IX_D-1) were obtained (98%). HPLC-MS (Condition A): rt=4.36 min; m/z: 511 [M+23]⁺.

rac-2,4,6-tri-O-Benzyl-1-O-(9-methoxynonyl)-myo-inositol (IX_D-2): According to general hydrolysis Procedure S, from 306 mg of (II_D-2), 198 mg of (IX_D-2) were obtained (68%). HPLC-MS (Condition A): rt=5.29 min; m/z: 607 [M+1]⁺, 629 [M+23]⁺.

rac-2,4,6-tri-O-Benzyl-1-O-(2-cyclopropylethyl)-myo-inositol (IX_D-3): According to general hydrolysis Procedure S, from 255 mg of (II_D-3), 181 mg of (IX_D-3) were obtained (74%). HPLC-MS (Condition A): rt=4.77 min; m/z: 519 [M+1]⁺.

rac-1-O-(5-Aminopentyl)-2,4,6-tri-O-benzyl-myo-inositol (IX_D-4): According to general azide reduction Procedure P, from 262 mg of (X_D-1), 198 mg of (IX_D-4) were obtained (79%). HPLC-MS (Condition A): rt=3.16 min; m/z: 536 [M+1]

rac-2,4,6-tri-O-Benzyl-1-O-(6,6,6-trifluorohexyl)-myo-inositol (IX_D-5): According to general hydrolysis Procedure S, from 107 mg of (II_D-5), 102 mg of (IX_D-5) were obtained (>99%). HPLC-MS (Condition A): rt=4.89 min; m/z: 589 [M+1]⁺.

rac-2,4,6-tri-O-Benzyl-1-O-(2-cyclopentylethyl)-myo-inositol (IX_D-6): According to general hydrolysis Procedure S, from 124 mg of (II_D-6), 118 mg of (IX_D-6) were obtained (>99%). HPLC-MS (Condition A): rt=5.28 min; m/z: 548 [M+1]⁺.

rac-1-O-(5-Acetamidopentyl)-2,4,6-tri-O-benzyl-myo-inositol (IX_D-7): According to general amide formation Procedure Q, from 112 mg of (IX_D-4) and 36 mg (1.1 eq.) of 2,5-dioxopyrrolidin-1-yl acetate, 107 mg of (IX_D-7) were obtained (89%). HPLC-MS (Condition A): rt=3.82 min; m/z: 579 [M+1]⁺.

rac-2,4,6-tri-O-Benzyl-1-O-(5-(4-(methoxymethyl)-1H-1,2,3-triazol-1-yl)pentyl)-myo-inositol (IX_D-8): According to general click reaction Procedure M, from 226 mg of (X_D-1) and 56 mg (2 eq.) of 3-methoxyprop-1-yne, 254 mg of (IX_D-8) were obtained (>99%). HPLC-MS (Condition A): rt=4.01 min; m/z: 633 [M+1]⁺.

rac-2,4,6-tri-O-Benzyl-2-O-propargyl-myo-inositol (IX_D-9): According to general hydrolysis Procedure S, from 164 mg of (II_D-4), 120 mg of (IX_D-9) were obtained (77%). HPLC-MS (Condition A): rt=4.24 min; m/z: 489 [M+1]⁺.

rac-2,4,6-tri-O-Benzyl-1-O-((1-(2-(metoxycarbonyl)ethyl)-1H-1,2,3-triazol-4-yl)methyl)-myo-inositol (IX_D-10): According to general click reaction Procedure L, from 185 mg of (IX_D-9) and 54 mg (1.1 eq.) of (38), 234 mg of (IX_D-10) were obtained (>99%). HPLC-MS (Condition A): rt=3.88 min; m/z: 618 [M+1]⁺.

### C.9 Intermediates X_D

rac-1-O-(5-Azidopentyl)-2,4,6-tri-O-benzyl-myo-inositol (X_D-1): According to general hydrolysis Procedure S, from 394 mg of (V_D-1), 263 mg of (X_D-1) were obtained (70%). HPLC-MS (Condition A): rt=4.74 min; m/z: 585 [M+23]⁺.

### D. Compound Characterization: Analytical and Spectroscopic Tests

### D.1. NMR

NMR spectra were recorded on an Agilent VNMRS-400 (¹H at 400.10 MHz and ³¹P at 162 MHz). In ¹H-NMR chemical shifts were expressed in ppm relative to TMS and coupling constant (J) in Hz. In ³¹P NMR no internal standard was used to collect the phosphorous NMR spectra. The usual internal standard is phosphoric acid, but this was not used due to concerns that it would affect the ¹H-NMR.

### D.2. HPLC-MS

Condition A: High-performance Liquid Chromatography (HPLC) 2795 Alliance Waters Aquity coupled to Detector DAD Agilent 1100 and Detector MS Waters ESI triple cuadrupolo Quattro micro, 10 μL of sample in MeOH was injected. Mass spectroscopy (MS) analyzed by FIA (flux injected analysis) coupled to LCT Premier Orthogonal Accelerated Time of Flight Mass Spectrometer, acquiring data by electrospray ionization (ESI) in positive mode. Spectra have been scanned between 50 and 1500 Da with values every 0.2 seconds and peaks are given m/z (% of basis peak). Stationary phase: ZORBAX Extend-C18 3.5 μm 2.1×50 mm (Ta 35 °C).

TABLE 2
Condition A, mobile phase

| | Mobile Phase A | Mobile Phase B |
|---|---|---|
| 1 | water + 0.05 | Acetonitrile + 0.05 |
| | formic acid | formic acid |

TABLE 3
Condition A, gradient

| | Time | Flow | | | |
|---|---|---|---|---|---|
| | (min) | (mL/min) | A (%) | B (%) | Curve |
| 1 | 0.0 | 0.50 | 95.0 | 5.0 | 5 |
| 2 | 0.50 | 0.50 | 95.0 | 5.0 | 5 |
| 3 | 5.50 | 0.50 | 0.0 | 100.0 | 5 |
| 4 | 7.50 | 0.50 | 0.0 | 100.0 | 5 |

Condition B: HPLC-MS were performed with a High-Performance Liquid Chromatography Thermo Ultimate 3000SD (Thermo Scientific Dionex) coupled to a photodiode array detector and a mass spectrometer LTQ XL ESI-ion trap (Thermo Scientific); 5 μL-20 μL of sample MeOH were injected (c=0.5 mg/mL). Data from mass spectra were analyzed by electrospray ionization in positive and negative mode and peaks are given m/z (% of basis peak). Stationary phase: ZORBAX Extend-C18 3.5 μm 2.1×50 mm (Ta 35 °C).

TABLE 4
Condition B, mobile phase

| | Mobile Phase A | Mobile Phase B |
|---|---|---|
| 1 | water + 0.05 | Acetonitrile + 0.05 |
| | formic acid | formic acid |

TABLE 5
Condition B, gradient

| | Time | Flow | | | |
|---|---|---|---|---|---|
| | (min) | (mL/min) | A (%) | B (%) | Curve |
| 1 | 0.0 | 0.50 | 95.0 | 5.0 | 5 |
| 2 | 0.50 | 0.50 | 95.0 | 5.0 | 5 |
| 3 | 5.50 | 0.50 | 0.0 | 100.0 | 5 |
| 4 | 7.50 | 0.50 | 0.0 | 100.0 | 5 |

Condition C: HPLC-MS were performed with a High-Performance Liquid Chromatography Thermo Ultimate 3000SD (Thermo Scientific Dionex) coupled to a photodiode array detector and a mass spectrometer LTQ XL ESI-ion trap (Thermo Scientific); 5 μL-20 μL of sample MeOH were injected (c=0.5 mg/mL). Data from mass spectra were analyzed by electrospray ionization in positive and negative mode and peaks are given m/z (% of basis peak). Stationary phase: Xbridge BEH Amide 2.5 μm 4.6×150 mm XP.

TABLE 6
Condition C, Mobile phase

| | Mobile Phase A | Mobile Phase B |
|---|---|---|
| 1 | H₂O + 0.1% DEA + | AcN |
| | 40 mM AcONH₄ | |

TABLE 7
Condition C, gradient

| | Time | Flow | | | |
|---|---|---|---|---|---|
| | (min) | (mL/min) | A (%) | B (%) | Curve |
| 1 | 0.0 | 1.0 | 5.0 | 95.0 | 5 |
| 2 | 10.00 | 1.0 | 60.0 | 40.0 | 5 |
| 3 | 15.00 | 1.0 | 60.0 | 40.0 | 5 |

Condition D: HPLC-MS were performed with a High-Performance Liquid Chromatography Thermo Ultimate 3000SD (Thermo Scientific Dionex) coupled to a photodiode array detector and a mass spectrometer LTQ XL ESI-ion trap (Thermo Scientific); 5 μL-20 μL of sample MeOH were injected (c=0.5 mg/mL). Data from mass spectra were analyzed by electrospray ionization in positive and negative mode and peaks are given m/z (% of basis peak). Stationary phase: ZORBAX Extend-C18 3.5 μm 2.1×50 mm (Ta 35 °C).

TABLE 8
Condition D, mobile phase

| | Mobile Phase A | Mobile Phase B |
|---|---|---|
| 1 | H₂O + 0.1% DEA + | AcN |
| | 40 mM AcONH₄ | |

TABLE 9
Condition D, gradient

| | Time | Flow | | | |
|---|---|---|---|---|---|
| | (min) | (mL/min) | A (%) | B (%) | Curve |
| 1 | 0.0 | 0.90 | 99.0 | 1.0 | 5 |
| 2 | 5.00 | 0.90 | 99.0 | 1.0 | 5 |
| 3 | 7.00 | 0.90 | 0.0 | 100.0 | 5 |
| 4 | 10.00 | 0.90 | 0.0 | 100.0 | 5 |

Condition E: HPLC-MS were performed with a High-Performance Liquid Chromatography Thermo Ultimate 3000SD (Thermo Scientific Dionex) coupled to a photodiode array detector and a mass spectrometer LTQ XL ESI-ion trap (Thermo Scientific); 5 μL-20 μL of sample water were injected (c=0.5 mg/mL). Data from mass spectra were analyzed by electrospray ionization in positive and negative mode and peaks are given m/z (% of basis peak). Stationary phase: Atlantis Premier BEH C18 AX 2.5 μm 2.1×100 mm, SN 1013130715504.

TABLE 10
Condition E, mobile phase

| | Mobile Phase A | Mobile Phase B |
|---|---|---|
| 1 | H₂O + 10 mM | AcN |
| | NH₄COOH | |

TABLE 11
Condition E, gradient

| | Time | Flow | | | |
|---|---|---|---|---|---|
| | (min) | (mL/min) | A (%) | B (%) | Curve |
| 1 | 0.0 | 0.50 | 100.0 | 0.0 | 1 |
| 2 | 2.00 | 0.50 | 100.0 | 0.0 | 5 |
| 3 | 8.00 | 0.50 | 0.0 | 100.0 | 5 |
| 4 | 12.00 | 0.50 | 0.0 | 100.0 | 5 |

Condition F: HPLC-MS were performed with a High-Performance Liquid Chromatography Thermo Ultimate 3000SD (Thermo Scientific Dionex) coupled to a photodiode array detector and a mass spectrometer LTQ XL ESI-ion trap (Thermo Scientific); 5 μL-20 μL of sample water were injected (c=0.5 mg/mL). Data from mass spectra were analyzed by electrospray ionization in positive and negative mode and peaks are given m/z (% of basis peak). Stationary phase: CORTECS T3 2.7 μm 2.1×150 mm, SN 1353131615509.

TABLE 12
Condition F, mobile phase

| | Mobile Phase A | Mobile Phase B |
|---|---|---|
| 1 | H₂O + 0.1% DEA + | AcN |
| | 40 mM AcONH₄ | |

TABLE 13
Condition E, gradient

| | Time | Flow | | | |
|---|---|---|---|---|---|
| | (min) | (mL/min) | A (%) | B (%) | Curve |
| 1 | 0.0 | 0.50 | 100.0 | 0.0 | 1 |
| 2 | 3.00 | 0.50 | 100.0 | 0.0 | 5 |
| 3 | 8.00 | 0.50 | 0.0 | 100.0 | 5 |
| 4 | 12.00 | 0.50 | 0.0 | 100.0 | 5 |

### Examples

### Compound 1 rac-4-O-Pentyl-myo-inositol-1,2,3,5,6-pentakis(phosphate) decasodium salt

According to general phosphate deprotection Procedure I, from 64 mg of (IV_A-1), 14.9 mg of Compound 1 (IA-1) were obtained (31% yield). ¹H NMR (400 MHz, D₂O) δ 4.99-4.91 (m, 1H), 4.42 (q, J=9.6 Hz, 1H), 4.15-3.95 (m, 3H), 3.88-3.81 (m, 1H), 3.79-3.71 (m, 2H), 3.64 (m, 3H), 1.65 (p, 7.2 Hz, 2H), 1.35-1.29 (m, 4H), 0.88 (m, 7.2 Hz, 3H). ³¹P NMR (162 MHz, D₂O) δ 3.94, 3.26. HPLC-MS (Condition C): rt=10.02 min; m/z: 797 [M+1+DEA]⁺, 870 [M+1+2DEA]⁺.

### Compound 2 rac-4-O-(5-Hydroxypentyl)-myo-inositol-1,2,3,5,6-pentakis(phosphate) decasodium salt

According to general phosphate deprotection Procedure I, from 30 mg of (IV_A-2), 8.24 mg of Compound 2 (IA-2) were obtained (39% yield). ¹H NMR (400 MHz, D₂O) δ 5.10-5.01 (m, 1H), 4.50-4.39 (m, 1H), 4.18-4.01 (m, 3H), 3.92-3.85 (m, 1H), 3.81-3.71 (m, 2H), 3.61 (t, J=6.8 Hz, 2H), 1.70-1.63 (m, 2H), 1.62-1.55 (m, 2H), 1.45-1.38 (m, 2H). ³¹P NMR (162 MHz, D₂O) δ 4.76, 2.86, 2.54, −0.16. HPLC-MS (Condition C): rt=10.36 min; m/z: 813 [M+1+DEA]⁺, 886 [M+1+2DEA]⁺.

### Compound 3 rac-4-O-(5-Methoxypentyl)-myo-inositol-1,2,3,5,6-pentakis(phosphate) decasodium salt

According to general phosphate deprotection Procedure I, from 97 mg of (IV_A-3), 20.5 mg Compound 3 (IA-3) were obtained (28% yield). ¹H NMR (400 MHz, D₂O) δ 5.00-4.96 (m, 1H), 4.52-4.42 (m, 1H), 4.23-4.61 (m, 3H), 3.86-3.72 (m, 3H), 3.51 (t, J=6.8 Hz, 2H), 3.35 (s, 3H), 1.70-1.60 (m, 4H), 1.41-1.34 (m, 2H). HPLC-MS (Condition C): rt=10.13 min; m/z: 827 [M+1+DEA]⁺, 900 [M+1+2DEA]⁺.

### Compound 4 rac-4-O-(5-Carboxypentyl)-myo-inositol-1,2,3,5,6-pentakis(phosphate) undecasodium salt

According to general phosphate deprotection Procedure J, from 23 mg of (IV_A-4), 4.4 mg of Compound 4 (IA-4) were obtained (25% yield). ¹H NMR (400 MHz, D₂O) δ: 5.08 (s, 1H), 5.51-4.43 (m, 1H), 4.25-3.97 (m, 3H), 3.86-3.72 (m, 4H), 2.20 (t, J=7.6 Hz, 2H), 1.71-1.64 (m, 2H), 1.562-1.55 (m, 2H), 1.36-1-32 (m, 2H). HPLC-MS (Condition C): rt=10.46 min; m/z: 841 [M+1+DEA]⁺, 914 [M+1+2DEA]⁺.

### Compound 5 rac-4-O-(2-(4-Acetylpiperazin-1-yl)ethyl)-myo-inositol-1,2,3,5,6-pentakis(phosphate) decasodium salt

According to general phosphate deprotection Procedure I, from 31 mg of (IV_A-5), 2.8 mg of Compound 5 (IA-5) were obtained (12% yield). ¹H NMR (400 MHz, D₂O) δ 4.93-4.86 (m, 1H), 4.45 (q, J=9.6 Hz, 1H), 4.28-4.20 (m, 2H), 4.18-4.11 (m, 4H), 4.06 (q, J=9.6 Hz, 2H), 4.01-3.88 (m, 2H), 3.88 (t, J=9.6 Hz, 2H), 3.44-3.29 (m, 4H), 2.18 (s, 3H). HPLC-MS (Condition C): rt=10.28 min; m/z: 808 [M+1+DEA]⁺, 881 [M+1+2DEA]⁺, 954[M+1+3DEA]⁺.

### Compound 6 2-O-Pentyl-myo-inositol-1,3,4,5,6-pentakis(phosphate) decasodium salt

According to general phosphate deprotection Procedure I, from 325 mg of (IV_B-1), 88.56 mg of Compound 6 (I_B-1) were obtained (36% yield). ¹H NMR (400 MHz, D₂O) δ 4.36 (m, 3H), 4.08 (q, J=9.6 Hz, 1H), 4.00 (t, J=9.6 Hz, 2H), 3.92 (t, J=7.2 Hz, 2H), 1.63 (p, J=7.2 Hz, 2H), 1.37-1.30 (m, 4H), 0.96-0.80 (m, 3H). ³¹P NMR (162 MHz, D₂O) δ 3.28, 2.32, 1.45. HPLC-MS (Condition C): rt=10.23 min; m/z: 724 [M+1]⁺, 797 [M+1+DEA]⁺, 870 [M+1+2DEA]⁺.

### Compound 7 2-O-(5-Methoxypentyl)-myo-inositol-1,3,4,5,6-pentakis(phosphate) decasodium salt

According to general phosphate deprotection Procedure I, from 71 mg of (IV_B-2), 6.7 mg of Compound 7 (I_B-2) were obtained (12.5% yield). ¹H NMR (400 MHz, D₂O) δ 4.38 (q, J=9.5 Hz, 2H), 4.20 (br, 1H), 4.13-4-05 (m, 3H), 3.90 (t, J=7.2 Hz, 2H), 3.51 (t, J=6.8 Hz, 2H), 3.52 (s, 3H), 1.67 (p, J=7.2 Hz, 2H), 1.62 (p, J=7.2 Hz, 2H), 1.40 (p, J=7.2 Hz 2H). ³¹P NMR (162 MHz, D₂O) δ 1.66 (br), 1.23 (br). HPLC-MS (Condition C): rt=10.24 min; m/z: 827 [M+1+DEA]⁺, 900 [M+1+2DEA]⁺.

### Compound 8 2-O-(9-Methoxynonyl)-myo-inositol-1,3,4,5,6-pentakis(phosphate) decasodium salt

According to general phosphate deprotection Procedure I, from 114 mg of (IV_B-3), 10 mg of Compound 8 (I_B-3) were obtained (11.4% yield). ¹H NMR (400 MHz, D₂O) δ 4.24 (q, J=8.8 Hz, 2H), 4.02 (s, 1H), 3.95 (t, J=9.7 Hz, 3H), 3.71 (t, J=6.8 Hz, 2H), 3.33 (t, J=6.8 Hz, 2H), 3.18 (s, 3H), 1.48 (t, J=6.8 Hz, 2H), 1.42 (q, J=6.8 Hz, 2H), 1.17 (m, 10H). ³¹P NMR (162 MHz, D₂O) δ 1.45, 1.24, 0.71. HPLC-MS (Condition D): rt=0.64 min; m/z: 737 [M+1]⁺, 810 [M+1+DEA]⁺, 883 [M+1+2DEA]⁺.

### Compound 9 2-O-(19-methoxynonadecyl)-myo-inositol-1,3,4,5,6-pentakis(phosphate) decasodium salt

According to general phosphate deprotection Procedure I, from 35 mg of (IV_B-4), 13.5 mg of Compound 9 (I_B-4) were obtained (43% yield). ¹H NMR (400 MHz, D₂O) δ 4.20 (q, J=8.8 Hz, 2H), 4.08 (s, 1H), 3.97-3.83 (m, 3H), 3.73 (t, J=7.5 Hz, 2H), 3.32 (t, J=6.7 Hz, 2H), 3.18 (s, 3H), 1.50-1.39 (m, 4H), 1.12 (br, 30H). ³¹P NMR (162 MHz, D₂O) δ 2.29, 1.56, 1.37. HPLC-MS (Condition D): rt=6.90 min; m/z: 1023 [M+1+2DEA]⁺, 1095 [M+1+3DEA]⁺.

### Compound 10 2-O-(29-methoxynonacosyl)-myo-inositol-1,3,4,5,6-pentakis(phosphate) decasodium salt

According to general phosphate deprotection Procedure I, from 18 mg of (IV_B-5), 5 mg of Compound 10 (I_B-5) were obtained (34% yield). ¹H NMR (400 MHz, D₂O) δ 4.25 (q, J=9.4 Hz, 2H), 4.03-3.92 (m, 4H), 3.72-3.66 (m, 2H), 3.32 (t, J=6.7 Hz, 2H), 3.18 (s, 3H), 1.51-1.38 (m, 4H), 1.17-1.11 (m, 50H). ³¹P NMR (162 MHz, D₂O) δ 1.47, 0.91, −0.25.

### Compound 11 rac-4-O-(3-(p-Tolyl)propyl)-myo-inositol-1,2,3,5,6-pentakis(phosphate) decasodium salt

According to general phosphate deprotection Procedure I, from 44 mg of (IV_A-6), 10 mg of Compound 11 (IA-6) were obtained (30% yield). ¹H NMR (400 MHz, D₂O) δ 7.19 (d, J=7.8 Hz, 2H), 7.11 (d, J=7.8 Hz, 2H), 4.43-3.83 (m, 2H), 3.81-3.66 (m, 2H), 2.59 (t, J=8 Hz, 2H), 2.22 (s, 3H), 1.90-1.78 (m, 2H). HPLC-MS (Condition C): rt=9.90 min; m/z: 713 [M+1]⁺, 785 [M+1+DEA]⁺, 759 [M+1+2DEA]⁺, 932 [M+1+3DEA]⁺.

### Compound 12 rac-4-O-(4-Methylpentyl)-myo-inositol-1,2,3,5,6-pentakis(phosphate) decasodium salt

According to general phosphate deprotection Procedure I, from 47 mg of (IV_A-7), 10.2 mg of Compound 12 (IA-7) were obtained (30% yield). δ 1H NMR (400 MHz, D₂O) δ 4.57-3.85 (m, 6H), 3.71-3.60 (m, 2H), 1.63-1.52 (m, 2H), 1.52-1.43 (m, 1H), 1.13-1.07 (m, 2H), 0.79 (d, J=6.6 Hz, 6H). HPLC-MS (Condition E): rt=10.13 min; m/z: 665 [M+1]⁺, 738 [M+1+DEA]⁺, 811 [M+1+2DEA]⁺, 884 [M+1+3DEA]⁺.

### Compound 13 rac-4-O-(6,6,6-Trifluorohexyl)-myo-inositol-1,2,3,5,6-pentakis(phosphate) decasodium salt

According to general phosphate deprotection Procedure I, from 48 mg of (IV_A-8), 3.8 mg of Compound 13 (IA-8) were obtained (10% yield). ¹H NMR (400 MHz, D₂O) δ 4.58-3.86 (m, 6H), 3.78-3.59 (m, 2H), 2.17-2.05 (m, 2H), 1.60 (p, J=7.6 Hz, 2H), 1.50 (p, J=7.6 Hz, 2H), 1.35 (p, J=7.6 Hz, 2H). HPLC-MS (Condition C): rt=10.49 min; m/z: 865 [M+1+2DEA]⁺.

### Compound 14 rac-4-O-(3-(4-Methoxyphenyl)propyl)-myo-inositol-1,2,3,5,6-pentakis(phosphate) decasodium salt

According to general phosphate deprotection Procedure I, from 48 mg of (IV_A-9), 4.1 mg of Compound 14 (IA-9) were obtained (11% yield). ¹H NMR (400 MHz, D₂O) δ 7.23 (d, J=8.6 Hz, 2H), 6.88 (d, J=8.6 Hz, 2H), 4.46-3.80 (m, 6H), 3.79-3.68 (m, 2H), 2.58 (t, J=8.0 Hz, 2H), 1.85 (p, J=8.0 Hz, 2H). HPLC-MS (Condition C): rt=10.11 min; m/z: 729 [M+1]⁺, 802 [M+1+DEA]⁺, 875 [M+1+2DEA]⁺.

### Compound 15 rac-4-O-(3-(3-(Trifluoromethyl)phenyl)propyl)-myo-inositol-1,2,3,5,6-pentakis(phosphate) decasodium salt

According to general phosphate deprotection Procedure I, from 35 mg of (IV_A-10), 8.6 mg of Compound 15 (IA-10) were obtained (32% yield). ¹H NMR (400 MHz, D₂O) δ 7.59 (s, 1H), 7.53 (d, J=7.8 Hz, 1H), 7.46 (d, J=7.8 Hz, 1H), 7.41 (t, J=7.8 Hz, 1H), 4.50-3.89 (m, 5H), 3.87-3.72 (m, 3H), 2.71 (t, J=8.0 Hz, 2H), 1.95-1.83 (m, 2H). HPLC-MS (Condition C): rt=9.55 min; m/z: 767 [M+1]⁺, 840 [M+1+DEA]⁺, 913 [M+1+2DEA]⁺.

### Compound 16 rac-4-O-(2-Cyclopentylethyl)-myo-inositol-1,2,3,5,6-pentakis(phosphate) decasodium salt

According to general phosphate deprotection Procedure I, from 33 mg of (IV_A-11), 6.6 mg of Compound 16 (IA-11) were obtained 26% yield). ¹H NMR (400 MHz, D₂O) δ 4.49-3.81 (m, 6H), 3.79-3.61 (m, 2H), 1.73-1.64 (m, 2H), 1.63-1.55 (m, 2H), 1.52-1.45 (m, 2H), 1.43-1.36 (m, 2H), 1.22-1.16 (m, 1H), 1.14-099 (m, 2H). HPLC-MS (Condition C): rt=10.34 min; m/z: 677 [M+1]⁺, 823 [M+1+2DEA]⁺.

### Compound 17 rac-4-O-(2-Cyclopropylethyl)-myo-inositol-1,2,3,5,6-pentakis(phosphate) decasodium salt

According to general phosphate deprotection Procedure I, from 46 mg of (IV_A-12), 9.5 mg of Compound 17 (IA-12) were obtained (27% yield). ¹H NMR (400 MHz, D₂O) δ 4.55-3.84 (m, 6H), 3.77-3.71 (m, 2H), 1.44 (q, J=7.3 Hz, 2H), 0.74-0.59 (m, 1H), 0.32-0.21 (m, 2H), 0.10-−0.10 (m, 2H). HPLC-MS (Condition C): rt=10.77 min; m/z: 649 [M+1]⁺, 795 [M+1+2DEA]⁺.

### Compound 18 rac-4-O-(5-(1H-Pyrazol-1-yl)pentyl)-myo-inositol-1,2,3,5,6-pentakis(phosphate) decasodium salt

According to general phosphate deprotection Procedure I, from 100 mg of (IV_A-13), 18.8 mg of Compound 18 (IA-13) were obtained (25% yield). ¹H NMR (400 MHz, D₂O) δ 7.60 (dd, J=2.2, 0.8 Hz, 1H), 7.46 (dd, J=2.2, 0.8 Hz, 1H), 6.24 (t, J=2.2 Hz, 1H), 4.63-3.89 (m, 6H), 4.08 (t, J=7.2 Hz, 2H), 3.73-3.56 (m, 2H), 1.76 (p, J=7.2 Hz, 2H), 1.62-1.51 (m, 2H), 1.18 (p, J=7.2 Hz, 2H). HPLC-MS (Condition C): rt=10.13 min; m/z: 717 [M+1]⁺, 790 [M+1+DEA]⁺, 863 [M+1+2DEA]⁺.

### Compound 19 rac-4-O-(5-Acetamidopentyl)-myo-inositol-1,2,3,5,6-pentakis(phosphate) decasodium salt

According to general phosphate deprotection Procedure I, from 14 mg of (IV_A-14), 3.1 mg of Compound 19 (IA-14) were obtained (29% yield). ¹H NMR (400 MHz, D₂O) δ 4.55-3.84 (m, 6H), 3.70-3.47 (m, 2H), 3.09 (t, J=6.8 Hz, 2H), 1.90 (s, 3H), 1.55-1.42 (m, 2H), 1.25-1.18 (m, 4H). HPLC-MS (Condition E): rt=4.88 min; m/z: 781 [M+1+DEA]⁺, 854 [M+1+2DEA]⁺.

### Compound 20 rac-4-O-(10-Carboxyldecyl)-myo-inositol-1,2,3,5,6-pentakis(phosphate) undecasodium salt

According to general phosphate deprotection Procedure J, from 7.5 mg of (IV_A-15), 3.4 mg of Compound 20 (IA-15) were obtained (59% yield). ¹H NMR (400 MHz, D₂O) δ 4.58-3.76 (m, 6H), 3.78-3.58 (m, 2H), 2.08 (t, J=7.6 Hz, 2H), 1.61-1.52 (m, 2H), 1.45 (t, J=7.6 Hz, 2H), 1.21 (br, 12H). HPLC-MS (Condition E): rt=5.20 min; m/z: 838 [M+1+DEA]⁺, 911 [M+1+2DEA]⁺, 984 [M+1+3DEA]⁺.

### Compound 21 rac-4-O-(10-Hydroxydecyl)-myo-inositol-1,2,3,5,6-pentakis(phosphate) decasodium salt

According to general phosphate deprotection Procedure I, from 12.9 mg of (IV_A-16), 2.8 mg of Compound 21 (IA-16) were obtained (31% yield). ¹H NMR (400 MHz, D₂O) δ 4.49-3.74 (m, 6H), 3.72-3.63 (m, 2H), 3.51 (t, J=6.8 Hz, 2H), 1.60-1.53 (m, 2H), 1.45 (t, J=6.8 Hz, 2H), 1.22 (br, 12H). HPLC-MS (Condition F): rt=6.27 min; m/z: 810 [M+1+DEA]⁺, 883 [M+1+2DEA]⁺.

### Compound 22 rac-4-O-Methyl-myo-inositol-1,2,3,5,6-pentakis(phosphate) decasodium salt

According to general phosphate deprotection Procedure I, from 10 mg of (IV_A-17), 0.6 mg of Compound 22 (IA-17) were obtained (8% yield). ¹H NMR (400 MHz, D₂O) δ 4.49-3.77 (m, 6H), 3.52 (s, 3H). HPLC-MS (Condition E): rt=2.26 min; m/z: 668 [M+1+DEA]⁺, 741 [M+1+2DEA]⁺, 814 [M+1+3DEA]⁺.

### Compound 23 rac-4-O-(7-Methoxyheptyl)-myo-inositol-1,2,3,5,6-pentakis(phosphate) decasodium salt

According to general phosphate deprotection Procedure I, from 30 mg of (IV_A-18), 9.7 mg of Compound 23 (IA-18) were obtained (42% yield). ¹H NMR (400 MHz, D₂O) δ 4.45-3.79 (m, 5H), 3.77-3.60 (m, 3H), 3.40 (t, J=6.8 Hz, 2H), 3.26 (s, 3H), 1.66-1.38 (m, 4H), 1.25 (br, 6H). HPLC-MS (Condition C): rt=10.98 min; m/z: 855 [M+1+2DEA]⁺.

### Compound 24 rac-4-O-Propyl-myo-inositol-1,2,3,5,6-pentakis(phosphate) decasodium salt

According to general phosphate deprotection Procedure I, from 20 mg of (IV_A-19), 1.5 mg of Compound 24 (IA-19) were obtained (10% yield). ¹H NMR (400 MHz, D₂O) δ 4.40-3.60 (m, 8H), 1.60, −1-51 (m, 2H), 0.81 (t, J=7.2 Hz, 3H). HPLC-MS (Condition E): rt=3.49 min; m/z: 696 [M+1+DEA]⁺, 769 [M+1+2DEA]⁺, 842 [M+1+3DEA]⁺.

### Compound 25 rac-4-O-(3-(4-(2-carboxyethyl)-1H-1,2,3-triazol-1-yl)propyl)-myo-inositol-1,2,3,5,6-pentakis(phosphate) undecasodium salt

According to general phosphate deprotection Procedure J, from 14 mg of (IV_A-20), 3.0 mg of Compound 25 (IA-20) were obtained (29% yield). ¹H NMR (400 MHz, D₂O) δ 7.81 (s, 1H), 4.53-4.44 (m, 2H), 4.39-3.60 (m, 8H), 2.84 (t, J=8 Hz, 2H), 2.44 (t, J=8 Hz, 2H), 2.16-2.05 (m, 2H). HPLC-MS (Condition E): rt=4.73 min; m/z: 762 [M+1]⁺, 835 [M+1+DEA]⁺, 908 [M+1+2DEA]⁺, 981 [M+1+3DEA]⁺.

### Compound 26 rac-4-O-(6-(4-(carboxy)-1H-1,2,3-triazol-1-yl)hexyl)-myo-inositol-1,2,3,5,6-pentakis(phosphate) undecasodium salt

According to general phosphate deprotection Procedure J, from 15 mg of (IV_A-21), 1.9 mg of Compound 26 (IA-21) were obtained (16% yield). ¹H NMR (400 MHz, D₂O) δ 8.13 (s, 1H), 4.35 (t, J=7.2 Hz, 2H), 4.39-3.43 (m, 8H), 1.88-1.78 (m, 2H), 1.62-1.54 (m, 2H), 1.36-1.23 (m, 4H). HPLC-MS (Condition E): rt=4.94 min; m/z: 849 [M+1+DEA]⁺, 922 [M+1+2DEA]⁺, 994 [M+1+3DEA]⁺.

### Compound 27 rac-4-O-(3-Aminopropyl)-myo-inositol-1,2,3,5,6-pentakis(phosphate) decasodium salt

According to general phosphate deprotection Procedure I, from 15 mg of (VI_A-1), 1.9 mg of Compound 27 (IA-22) were obtained (17% yield). ¹H NMR (400 MHz, D₂O) δ 4.40-3.80 (m, 6H), 3.08 (t, J=6.4 Hz, 2H), 1.94-1.88 (m, 2H). HPLC-MS (Condition E): rt=1.63 min; m/z: 638 [M+1]⁺, 711 [M+1+DEA]⁺, 784 [M+1+2DEA]⁺.

### Compound 28 rac-4-O-(6-Aminohexyl)-myo-inositol-1,2,3,5,6-pentakis(phosphate) decasodium salt

According to general phosphate deprotection Procedure I, from 44 mg of (VI_A-2), 15.6 mg of Compound 28 (IA-23) were obtained (48% yield). ¹H NMR (400 MHz, D₂O) δ 4.40-3.80 (m, 6H), 3.55-3.65 (m, 2H), 2.92 (dd, J=7.6, 5.6 Hz, 2H), 1.70-1.40 (m, 4H), 1.28-1.13 (m, 4H). HPLC-MS (Condition C): rt=9.23 min; m/z: 752 [M+1+DEA]⁺, 825 [M+1+2DEA]⁺.

### Compound 29 5-O-Propyl-myo-inositol-1,2,3,4,6-pentakis(phosphate) decasodium salt

According to general phosphate deprotection Procedure I, from 10 mg of (IV_C-1), 3.3 mg of Compound 29 (IC-1) were obtained (45% yield). ¹H NMR (400 MHz, D₂O) δ 4.64-4.56 (m, 1H), 4.38 (q, J=9.6 Hz, 2H), 4.02-3.93 (m, 2H), 3.71 (t, J=7.4 Hz, 2H), 3.34 (t, J=9.6 Hz, 1H), 1.58 (q, J=7.4 Hz, 2H), 0.81 (t, J=7.4 Hz, 3H). HPLC-MS (Condition E): rt=3.41 min; m/z: 696 [M+1+DEA]⁺, 769 [M+1+2DEA]⁺, 842 [M+1+3DEA]⁺.

### Compound 30 5-O-(2-Cyclopropylethyl)-myo-inositol-1,2,3,4,6-pentakis(phosphate) decasodium salt

According to general phosphate deprotection Procedure I, from 10 mg of (IV_C-2), 3.53 mg of Compound 30 (IC-2) were obtained (47% yield). ¹H NMR (400 MHz, D₂O) δ 4.36 (q, J=9.6 Hz, 2H), 3.99-3.91 (m, 2H), 3.81 (t, J=7.6 Hz, 2H), 3.65-3.55 (m, 1H), 3.36-3.26 (m, 1H), 1.47 (q, J=7.6 Hz, 2H), 0.70-061 (m, 1H), 0.32-0.26 (m, 2H), 0.01-−0.04 (m, 2H). HPLC-MS (Condition E): rt=4.96 min; m/z: m/z: 722 [M+1+DEA]⁺, 795 [M+1+2DEA]⁺, 868 [M+1+3DEA]⁺.

### Compound 31 5-O-(9-Methoxynonyl)-myo-inositol-1,2,3,4,6-pentakis(phosphate) decasodium salt

According to general phosphate deprotection Procedure I, from 15 mg of (IV_C-3), 6.7 mg of Compound 31 (IC-3) were obtained (58% yield). ¹H NMR (400 MHz, D₂O) δ 4.54-4.46 (m, 2H), 4.44-4.15 (m, 3H), 3.84 (br, 1H), 3.66 (t, J=7.6 Hz, 2H), 3.40 (t, J=6.8 Hz, 2H), 3.26 (s, 3H), 1.63-1.53 (m, 2H), 1.53-1.43 (m, 2H), 1.23 (br, 10H). HPLC-MS (Condition E): rt=5.95 min; m/z: m/z: 810 [M+1+DEA]⁺, 883 [M+1+2DEA]⁺, 956 [M+1+3DEA]⁺.

### Compound 32 rac-1-O-Propyl-myo-inositol-2,3,4,5,6-pentakis(phosphate) decasodium salt

According to general phosphate deprotection Procedure I, from 53 mg of (IV_D-1), 13.28 mg of Compound 32 (I_D-1) were obtained (34% yield). ¹H NMR (400 MHz, D₂O) δ 4.60-3.80 (m, 6H), 3.70-3.52 (m, 2H), 1.66-1.53 (m, 2H), 0.81 (t, J=7.6 Hz, 3H). HPLC-MS (Condition E): rt=3.65 min; m/z: 696 [M+1+DEA]⁺, 769 [M+1+2DEA]⁺, 842 [M+1+3DEA]⁺.

### Compound 33 rac-4-O-(6-Amino-6-oxohexyl)-myo-inositol-1,2,3,5,6-pentakis(phosphate) decasodium salt

According to general phosphate deprotection Procedure I, from 6 mg of (IV_A-22), 1.42 mg of Compound 33 (IA-24) were obtained (31% yield). ¹H NMR (400 MHz, D₂O) δ 4.36 (q, J=9.2 Hz, 1H), 4.06 (q, J=9.2 Hz, 1H), 4.00 (d, J=8.0 Hz, 1H), 3.94 (t, J=9.2 Hz, 1H), 3.78 (q, J=8.0 Hz, 1H), 3.72-3.67 (m, 1H) 3.67 (t, J=6.8 Hz, 2H), 2.21 (t, J=6.8 Hz, 1H), 1.58 (p, J=6.8 Hz, 2H), 1.55 (p, J=6.8 Hz, 2H), 1.32 (p, J=6.8 Hz, 2H). HPLC-MS (Condition E): rt=2.25 min; m/z: 694 [M+1]⁺, 767 [M+1+DEA]⁺, 840 [M+1+2DEA]⁺.

### Compound 34 2-O-Propyl-myo-inositol-1,3,4,5,6-pentakis(phosphate) decasodium salt

According to general phosphate deprotection Procedure I, from 30 mg of (IV_B-6), 5 mg of Compound 34 (I_B-6) were obtained (23% yield). ¹H NMR (400 MHz, D₂O) δ 4.80-4.70 (m, 1H) 4.55-4.43 (m, 2H), 4.24 (brs, 1H), 4.22-4.13 (m, 2H), 3.84 (t, J=7.2 Hz, 2H), 1.64 (q, J=7.2 Hz, 2H), 0.94 (t, J=7.2 Hz, 3H). HPLC-MS (Condition E): rt=4.85 min; m/z: 623 [M+1]⁺, 796 [M+1+DEA]⁺, 769 [M+1+2DEA]⁺.

### Compound 35 2-O-(5-Acetamidopentyl)-myo-inositol-1,3,4,5,6-pentakis(phosphate) decasodium salt

According to general phosphate deprotection Procedure I, from 12 mg of (IV_B-13), 8 mg of Compound 35 (I_B-7) were obtained (89% yield). ¹H NMR (400 MHz, D₂O) δ 4.80-4.70 (m, 1H), 4.48-4.31 (m, 2H), 4.13-3.87 (m, 5H), 3.86-3.74 (m, 1H), 3.19 (t, J=6.7 Hz, 2H), 1.99 (s, 3H), 1.72-1.61 (m, 2H), 1.56 (q, J=7.6 Hz, 2H), 1.46-1.37 (m, 2H). HPLC-MS (Condition E): rt=4.97 min; m/z: 781 [M+1+DEA]⁺, 854 [M+1+2DEA]⁺.

### Compound 36 2-O-(6,6,6-Trifluorohexyl)-myo-inositol-1,3,4,5,6-pentakis(phosphate) decasodium salt

According to general phosphate deprotection Procedure I, from 58 mg of (IV_B-8), 8.5 mg of Compound 36 (I_B-8) were obtained (20% yield). ¹H NMR (400 MHz, D₂O) 4.80-4.70 (m, 1H), δ 4.45-4.30 (m, 2H), 4.15-3.86 (m, 4H), 3.86-3.70 (m, 1H), 2.32-2.08 (m, 2H), 1.67 (p, J=7.6 Hz, 2H), 1.60 (p, J=7.6 Hz, 2H), 1.47 (p, J=7.6 Hz, 2H). ³¹P NMR (162 MHz, D₂O) δ 3.23, 2.24, 1.51. HPLC-MS (Condition E): rt=4.85 min; m/z: 719 [M+1]⁺, 792 [M+1+DEA]⁺, 865 [M+1+2DEA]⁺, 938 [M+1+3DEA]⁺.

### Compound 37 2-O-(6-Amino-6-oxohexyl)-myo-inositol-1,3,4,5,6-pentakis(phosphate) decasodium salt

According to general phosphate deprotection Procedure I, from 24 mg of (IV_B-9), 4.2 mg of Compound 37 (I_B-9) were obtained (24% yield). ¹H NMR (400 MHz, D₂O) δ 4.73-4.83 (m, 1H), 4.43-4.31 (m, 2H), 4.15-3.99 (m, 3H), 3.96 (m, 2H), 2.31 (t, J=7.6 Hz, 2H), 1.66 (p, J=7.4 Hz, 4H), 1.48-1.42 (m, 2H). ³¹P NMR (162 MHz, D₂O) δ 2.96, 1.94, 1.39. HPLC-MS (Condition E): rt=1.77 min; m/z: 694 [M+1]⁺, 767 [M+1+DEA]⁺, 840 [M+1+2DEA]⁺, 913 [M+1+3DEA]⁺.

### Compound 38 2-O-(2-Cyclopentylethyl)-myo-inositol-1,3,4,5,6-pentakis(phosphate) decasodium salt

According to general phosphate deprotection Procedure I, from 124 mg of (IV_B-10), 7.2 mg of Compound 38 (I_B-10) were obtained (7.7% yield). ¹H NMR (400 MHz, D₂O) δ 4.92-4.65 (m, 2H), 4.57-4.28 (m, 2H), 4.18-3.87 (m, 3H), 3.86-3.74 (m, 1H), 1.89-1.74 (m, 3H), 1.73-1.64 (m, 2H), 1.64-1.53 (m, 2H), 1.53-1.41 (m, 2H), 1.20-1.10 (m, 2H). HPLC-MS (Condition E): rt=5.16 min; m/z: 677 [M+1]⁺, 750 [M+1+DEA]⁺, 823 [M+1+2DEA]⁺, 896 [M+1+3DEA]⁺.

### Compound 39 2-O-(2-Cyclopropylethyl)-myo-inositol-1,3,4,5,6-pentakis(phosphate) decasodium salt

According to general phosphate deprotection Procedure I, from 65 mg of (IV_B-11), 3.0 mg of Compound 39 (I_B-11) were obtained (4.7% yield). ¹H NMR (400 MHz, D₂O) δ 5.00-4.78 (m, 1H), 4.33-4.15 (m, 2H), 4.05-3.83 (m, 4H), 3.84-3.68 (m, 1H), 1.44 (q, J=7.2 Hz, 2H), 0.77-0.60 (m, 1H), 0.38-0.25 (m, 2H), 0.07-−0.08 (m, 2H). HPLC-MS (Condition E): rt=1.94 min; m/z: 649 [M+1]⁺, 722 [M+1+DEA]⁺, 795 [M+1+2DEA]⁺, 868 [M+1+3DEA]⁺.

### Compound 40 2-O-(5-Hydroxypentyl)-myo-inositol-1,3,4,5,6-pentakis(phosphate) decasodium salt

According to general phosphate deprotection Procedure I, from 73 mg of (IV_B-7), 13.6 mg of Compound 40 (I_B-12) were obtained (27% yield). ¹H NMR (400 MHz, D₂O) δ 5.21-4.73 (m, 2H), 4.60-4.17 (m, 2H), 4.40 (q, J=9.6 Hz, 1H), 4.08 (q, J=9.6 Hz, 1H), 4.03-3.89 (m, 1H), 3.89-3.70 (m, 1H), 3.63 (t, J=6.5 Hz, 2H), 1.75-1.59 (m, 4H), 1.48-144 (m, 2H). HPLC-MS (Condition E): rt=4.85 min; m/z: 667 [M+1]⁺.

### Compound 41 2-O-(5-(4-(Methoxymethyl)-1H-1,2,3-triazol-1-yl)pentyl)-myo-inositol-1,3,4,5,6-pentakis(phosphate) decasodium

According to general phosphate deprotection Procedure I, from 25 mg of (IV_B-12), 10.8 mg of Compound 41 (I_B-13) were obtained (56% yield). ¹H NMR (400 MHz, D₂O) δ 8.08 (s, 1H), 4.61 (s, 2H), 4.47 (t, J=7.2 Hz, 2H), 4.47-4.28 (m, 3H), 4.17-3.87 (m, 4H), 3.39 (s, 3H), 1.97 (p, J=7.7 Hz, 2H), 1.69 (p, J=7.7 Hz, 2H), 1.45-1.33 (m, 2H). ³¹P NMR (162 MHz, D₂O) δ 3.65, 2.66, 1.67. HPLC-MS (Condition E): rt=5.33 min; m/z: 907 [M+1+2DEA]⁺.

### Compound 42 2-O-((1-(2-Carboxyethyl)-1H-1,2,3-triazol-4-yl)methyl)-myo-inositol-1,3,4,5,6-pentakis(phosphate) undecasodium salt

According to general phosphate deprotection Procedure J, from 100 mg of (IV_B-14), 45 mg of Compound 42 (I_B-14) were obtained (57% yield). ¹H NMR (400 MHz, D₂O) δ 8.16 (s, 1H), 5.07 (s, 2H), δ 4.73-4.83 (m, 1H), 4.63 (t, J=7.2 Hz, 3H), 4.32 (brs, 3H), 4.04 (brs, 2H), 2.81 (t, J=7.2 Hz, 2H). HPLC-MS (Condition E): rt=1.16 min; m/z: 880 [M+1+2DEA]⁺, 953 [M+1+3DEA]⁺.

### Compound 43 2-O-Propargyl-myo-inositol-1,3,4,5,6-pentakis(phosphate) decasodium salt

According to general phosphate deprotection Procedure T, from 25 mg of (IV_B-6), 7.5 mg of Compound 43 (I_B-15) were obtained (40% yield). ¹H NMR (400 MHz, D₂O) δ 4.64 (s, 2H), 4.42-4.34 (m, 3H), 4.14-4.06 (m, 3H), 2.88 (t, J=2.4 Hz, 1H). ³¹P NMR (162 MHz, D₂O) δ 2.89, 1.95, 1.43. HPLC-MS (Condition E): rt=3.20 min; m/z: 693 [M+1+DEA]⁺, 766 [M+1+2DEA]⁺.

### Compound 44 5-O-(9-Methoxypentyl)-myo-inositol-1,2,3,4,6-pentakis(phosphate) decasodium salt

According to general phosphate deprotection Procedure I, from 6 mg of (IV_C-4), 1.7 mg of Compound 44 (IC-4) were obtained (37% yield). ¹H NMR (400 MHz, D₂O) δ 4.64-4.45 (m, 1H), 4.43-4.35 (m, 1H), 4.32-4.16 (m, 1H), 4.023-3.87 (m, 2H), 3.77-3.70 (m, 2H), 3.66-3.55 (m, 1H), 3.42 (t, J=6.8 Hz, 2H), 3.26 (s, 3H), 1.65-1.56 (m, 2H), 1.53 (iq, J=6.8 Hz, 2H), 1.20-1.23 (m, 2H). ³¹P NMR (162 MHz, D₂O) δ 3.64, 3.23, 2.63. HPLC-MS (Condition E): rt=4.67 min; m/z: 826 [M+1+2DEA]⁺, 899 [M+1+3DEA]⁺.

### Compound 45 5-O-(6,6,6-Trifluorohexyl)-myo-inositol-1,2,3,4,6-pentakis(phosphate) decasodium salt

According to general phosphate deprotection Procedure I, from 10 mg of (IV_C-5), 1 mg of Compound 45 (IC-5) were obtained (13% yield). ¹H NMR (400 MHz, D₂O) δ 4.58-4.16 (m, 3H), 4.02-3.90 (m, 2H), 3.80-3.71 (m, 2H), 3.39-3.22 (m, 1H), 2.23-2.02 (m, 2H), 1.67-1.56 (m, 2H), 1.51 (p, J=7.4 Hz, 2H), 1.38-1.30 (m, 2H). HPLC-MS (Condition E): rt=6.12 min; m/z: 865 [M+1+2DEA]⁺, 938 [M+1+3DEA]⁺.

### Compound 46 5-O-(5-(4-(Methoxymethyl)-1H-1,2,3-triazol-1-yl)pentyl)-myo-inositol-1,2,3,4,6-pentakis(phosphate) decasodium salt

According to general phosphate deprotection Procedure I, from 7 mg of (IV_C-6), 2.5 mg of Compound 45 (IC-6) were obtained (46% yield). ¹H NMR (400 MHz, D₂O) δ 7.99 (s, 1H), 4.53 (s, 2H), 4-50-4.35 (m, 1H), 4.37 (t, J=7.0 Hz, 2H), 4.06-3.84 (m, 3H), 3.78-3.68 (m, 2H), 3.30 (s, 3H), 1.86 (t, J=7.6 Hz, 2H), 1.69-1.58 (m, 2H), 1.33-1.14 (m, 4H). HPLC-MS (Condition E): rt=3.20 min; m/z: 762 [M+1]⁺.

### Compound 47 rac-1-O-(9-Methoxynonyl)-myo-inositol-2,3,4,5,6-pentakis(phosphate) decasodium salt

According to general phosphate deprotection Procedure I, from 100 mg of (IV_D-2), 44 mg of Compound 47 (I_D-2) were obtained (57% yield). ¹H NMR (400 MHz, D₂O) δ 4.65-4.39 (m, 4H), 4.29 (d, J=11.6 Hz, 1H), 4.18-3.94 (m, 1H), 3.85-3.73 (m, 1H), 3.72-3.65 (m, 1H), 3.47 (t, J=6.6 Hz, 2H), 3.33 (s, 3H), 1.71-1.61 (m, 2H), 1.56 (p, J=6.6 Hz, 2H), 1.30 (brs, 10H). HPLC-MS (Condition E): rt=5.45 min; m/z: 810 [M+1+DEA]⁺, 883 [M+1+2DEA]⁺.

### Compound 48 rac-1-O-(2-Cyclopropylethyl)-myo-inositol-2,3,4,5,6-pentakis(phosphate) decasodium salt

According to general phosphate deprotection Procedure I, from 90 mg of (IV_D-3), 15.4 mg of Compound 48 (I_D-3) were obtained (23% yield). ¹H NMR (400 MHz, D₂O) δ 4.64-4.32 (m, 5H), 4.22-4.09 (m, 1H), 3.78-3.65 (m, 2H), 1.49-1.41 (m, 2H), 0.72-0.59 (m, 1H), 0.34-0.27 (m, 2H), 0.04-−0.04 (m, 2H). HPLC-MS (Condition E): rt=4.92 min; m/z: 722 [M+1+DEA]⁺, 795 [M+1+2DEA]⁺, 868 [M+1+3DEA]⁺.

### Compound 49 rac-1-O-(5-Acetamidopentyl)-myo-inositol-2,3,4,5,6-pentakis(phosphate) decasodium salt

According to general phosphate deprotection Procedure I, from 10 mg of (IV_D-4), 1.7 mg of Compound 49 (I_D-4) were obtained (22% yield). ¹H NMR (400 MHz, D₂O) δ 4.39-4.22 (m, 2H), 4.10-4.00 (m, 1H), 3.97-3.86 (m, 1H), 3.73-3.66 (m, 1H), 3.54-3.46 (m, 1H), 3.09 (t, J=6.5 Hz, 2H), 1.90 (s, 3H), 1.62-1.52 (m, 2H), 1.49-1.41 (m, 2H), 1.37-1.29 (m, 2H). HPLC-MS (Condition E): rt=3.10 min; m/z: 781 [M+1+DEA]⁺, 854 [M+1+2DEA]⁺, 926 [M+1+3DEA]⁺.

### Compound 50 rac-1-O-(6,6,6-Trifluorohexyl)-myo-inositol-2,3,4,5,6-pentakis(phosphate) decasodium salt (CSC-san17-016)

According to general phosphate deprotection Procedure I, from 23 mg of (IV_D-5), 0.6 mg of Compound 50 (I_D-5) were obtained (3.4% yield). HPLC-MS (Condition E): rt=6.83 min; m/z: 719 [M+1]⁺, 865 [M+1+2DEA]⁺.

### Compound 51 rac-1-O-(2-Cyclopentylethyl)-myo-inositol-2,3,4,5,6-pentakis(phosphate) decasodium salt

According to general phosphate deprotection Procedure I, from 25 mg of (IV_D-6), 1.6 mg of Compound 51 (I_D-6) were obtained (8.5% yield). HPLC-MS (Condition E): rt=6.35 min; m/z: 677 [M+1]⁺, 750 [M+1+DEA]⁺, 823 [M+1+2DEA]⁺, 896 [M+1+3DEA]⁺.

### Compound 52 rac-1-O-(5-(4-(Methoxymethyl)-1H-1,2,3-triazol-1-yl)pentyl)-myo-inositol-2,3,4,5,6-pentakis(phosphate) decasodium salt

According to general phosphate deprotection Procedure I, from 10 mg of (IV_D-8), 6.3 mg of Compound 52 (I_D-7) were obtained (82% yield). ¹H NMR (400 MHz, D₂O) δ 8.07 (s, 1H), 4.61 (s, 2H), 4.51-4.41 (m, 1H), 4.46 (t, J=7.2 Hz, 2H), 4.36-4.24 (m, 1H), 4.16-3.88 (m, 1H), 3.84-3.52 (m, 3H), 3.39 (s, 3H), 2.00-1.92 (m, 2H), 1.87-1.61 (m, 2H), 1.48-1.26 (m, 2H). HPLC-MS (Condition E): rt=5.19 min; m/z: 835 [M+1+DEA]⁺, 908 [M+1+2DEA]⁺.

### Compound 53 rac-1-O-((1-(2-(Metoxycarbonyl)ethyl)-1H-1,2,3-triazol-4-yl)methyl)-myo-inositol-2,3,4,5,6-pentakis(phosphate) undecasodium salt

According to general phosphate deprotection Procedure J, from 104 mg of (IV_D-7), 44.1 mg of Compound 53 (I_D-8) were obtained (55% yield). ¹H NMR (400 MHz, D₂O) δ 8.01 (s, 1H), 4.94 (d, J=12.4 Hz, 1), 4.86 (d, J=12.4 Hz, 1H), 4.63-4.57 (m, 3H), 4.51-4.33 (m, 2H), 4.31-4.08 (m, 3H), 4.02-3.78 (m, 1H), 2.73 (t, J=7.1 Hz, 2H). HPLC-MS (Condition E): rt=1.21 min; m/z: 880 [M+1+2DEA]⁺, 953 [M+1+3DEA]⁺.

### Pharmacological Assays

### A. In Vitro Inhibition of Calcium Phosphate Crystallization

The in vitro efficacy of the IP5 substituted compounds (e.g., Compounds 1 to 32) of the invention on the inhibition of calcium phosphate crystallization in human plasma samples was evaluated according to a spectrophotometric assay previously described in the art (Ferrer M, et al., Sci Rep 2017; 7:6858, doi:10.1038/s41598-017-07203-x).

A 96-well plate was employed. Plasma was spiked (1 volume of the IP5 substituted compounds of the invention per 19 volumes of plasma) with increasing concentrations of the derivatives in the range of 0-100 μM. Plasma was then centrifuged at 10,000 g at rt and subsequently mixed with a mixture of 5 mM hydrogen phosphate and 41.67 mM calcium to attain final concentrations of 1.5 mM phosphate and 12.5 mM calcium, respectively. All reagent solutions were filtered, and pH adjusted to 7.4.

Crystallization of calcium phosphate was monitored spectrophotometrically for 30 min at rt by measuring the increments in absorbance at 550 nm using the Biotek Powerwave XS Microplate spectrophotometer (BioTek Instruments, Inc., Winooski, VT, US). The plate was incubated at rt in an orbital shaker and absorbance measured every 3 min.

Plasma crystallization was assessed based on slope measurement in the linear range between 6 and 24 min from plots of increase in absorbance versus logarithm of time. The efficacy of different myo-inositol phosphates derivatives in preventing in vitro formation of calcium phosphate crystals was assessed in human plasma samples using the slopes obtained between 6- and 24-min. Inhibition of crystallization was measured by comparing the slopes of the control sample (blank plasma) with those of samples containing the inhibitor as shown below:

$$\% Inhibition of crystallization = \frac{Slope ( blank plasma ) - Slope ( inhibitor )}{Slope ( blank plasma )} \cdot 100$$

See Table 14.

TABLE 14

| | Relative |
|---|---|
| Compound | Potency |
| 1 | +++ |
| 2 | +++ |
| 3 | +++ |
| 5 | +++ |
| 6 | +++ |
| 7 | ++ |
| 8 | ++ |
| 9 | + |
| 10 | +++ |
| 11 | ++ |
| 12 | ++ |
| 13 | +++ |
| 14 | +++ |
| 15 | ++ |
| 16 | +++ |
| 17 | ++ |
| 18 | ++ |
| 19 | ++ |
| 20 | + |
| 21 | + |
| 22 | ++ |
| 23 | ++ |
| 24 | +++ |
| 25 | + |
| 26 | + |
| 27 | +++ |
| 28 | +++ |
| 29 | ++ |
| 30 | ++ |
| 31 | ++ |
| 32 | +++ |
| 33 | +++ |
| 34 | ++ |
| 35 | + |
| 36 | +++ |
| 37 | ++ |
| 38 | ++ |
| 39 | ++ |
| 40 | ++ |
| 41 | + |
| 42 | ++ |
| 43 | ++ |
| 44 | ++ |
| 45 | ++ |
| 46 | + |
| 47 | +++ |
| 48 | +++ |
| 49 | ++ |
| 50 | ++ |
| 51 | +++ |
| 52 | ++ |
| 53 | ++ |

Legend:
+++-EC₅₀ ≤ 5 μM
++-5 μM < EC₅₀ ≤ 10 μM
+-EC₅₀ > 10 μM

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described examples but should be defined only in accordance with the following claims and their equivalents.

1. A compound of general formula I: 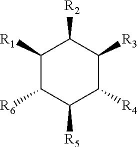
a pharmaceutically acceptable salt thereof, or a combination thereof, wherein
(i) R₁, R₂, R₃, R₅, and R₆ independently represent —OPO₃H₂ and R₄ is a substituent group of formula II or formula III, or R₁, R₂, R₃, R₄, and R₅ independently represent —OPO₃H₂ and R₆ is a substituent group of formula II or formula III, 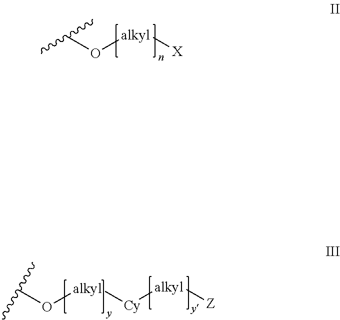
(ii) R₁, R₃, R₄, R₅, and R₆ independently represent —OPO₃H₂ and R₂ is a substituent group of formula II or formula III,
(iii) R₁, R₂, R₃, R₄, and R₆ independently represent —OPO3H2 and R₅ is a substituent group of formula II or formula III,
(iv) R₂, R₃, R₄, R₅, and R₆ independently represent —OPO₃H₂ and R₁ is a substituent group of formula II; or R₁, R₂, R₄, R₅, and R₆ independently represent —OPO₃H₂ and R₃ is a substituent group of formula II or formula III,
wherein, for formula II, n is an integer between 1 and 30, wherein the terminal group X is selected from the group consisting of —H, —OR, —NRR′, —COOR, —CONRR′, —NHCOR, —NHCOOR, —OCONR, —NHSO₂R, —NHCONRR′, halogen, —CF₃, alkyl, alkenyl, alkynyl, carbocycle, and heterocycle, and wherein R and R′ are H or an alkyl group, and wherein, for formula III, y and y′ are an integer between 0 and 10, wherein Cy is a cyclic linker, wherein the terminal group Z is selected from the group consisting of alkyl, —COR, —OR, —NRR′, —COOR, —CONRR′, —NHCOR, —NHCOOR, —OCONR —NHSO₂R, —NHCONRR′, halogen, and —CF₃, and wherein R and R′ are H or an alkyl group.

2. The compound according to claim 1, wherein the pharmaceutically acceptable salt is a sodium or magnesium salt.

3. The compound according to claim 3, wherein the sodium salt is a tetrasodium salt, pentasodium salt, hexasodium salt, heptasodium salt, octasodium salt, nonasodium salt, decasodium or undecasodium salt.

4. The compound according to any one of claims 1 to 3, wherein the compound is selected from the group consisting of Compound 1 to Compound 53, and any combination thereof.

5. The compound according to claim 4, wherein the compound is Compound 1 of chemical structure:

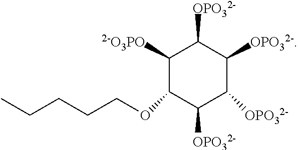

6. The compound according to claim 4, wherein the compound is Compound 2 of chemical structure:

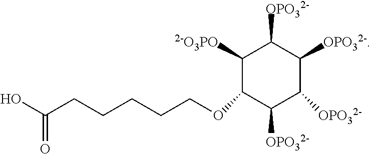

7. The compound according to claim 3, wherein the compound is Compound 16 of chemical structure:

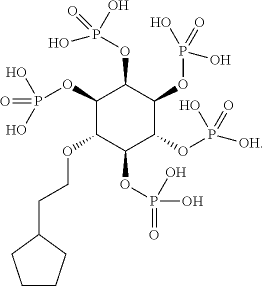 [SMILES: O=P(O)(O)O[C@@H]1[C@H](OP(=O)(O)O)[C@H](OP(=O)(O)O)[C@@H](OCCC2CCCC2)[C@H](OP(=O)(O)O)[C@H]1OP(=O)(O)O]

8. A pharmaceutical composition comprising a compound according to any one of claims 1 to 7 and at least one pharmaceutically acceptable excipient.

9. A method to inhibit the formation or growth of a calcium salt/crystal in a subject in need thereof comprising administering to the subject an effective amount of a compound of formula I according to any one of claims 1 to 7, or a pharmaceutical composition according to claim 8.

10. A method to inhibit the formation or growth of a calcium salt/crystal according to claim 9, wherein calcium salt/crystal is a calcium phosphate.

11. A method to inhibit the formation or growth of a calcium salt/crystal according to claim 10, wherein the calcium phosphate is hydroxyapatite.

12. A method to treat or prevent a disease or condition associated with pathological crystallization in a subject in need thereof comprising administering to the subject an effective amount of a compound of formula I according to any one of claims 1 to 7, or a pharmaceutical composition according to claim 8.

13. A method to inhibit the progression of a crystallization process in a subject in need thereof comprising administering to the subject an effective amount of a compound of formula I according to any one of claims 1 to 7, or a pharmaceutical composition according to claim 8.

14. The methods according to any one of claims 9 to 13, wherein the subject is human.

15. The methods according to any one of claims 9 to 14, wherein the administration is topical, enteral or parenteral.

16. The methods according to claim 15, wherein the parenteral administration is intravenous or subcutaneous.

17. A compound selected from the group consisting of the compounds listed in Table 1.

18. A kit or article of manufacture comprising at least one compound according to any one of claims 1 to 7, a pharmaceutical composition according to claim 8, or a compound according to claim 17.

19. A process of making a compound of formula I according to any one of claims 1 to 7 comprising the use of at least one compound according to claim 17.
